(19) United States
(12) Patent Application Publication
(10) Pub. No.: US 2026/0166347 A1
(43) Pub. Date: Jun. 18, 2026

(54) BRAIN MODULATION SYSTEMS AND METHODS

(71) Applicant: Spire Therapeutics, Inc., Salt Lake City, UT (US)

(72) Inventor: Jan Kubanek, Salt Lake City, UT (US)

(21) Appl. No.: 19/536,016

(22) Filed: Feb. 10, 2026

Related U.S. Application Data
(63) Continuation of application No. PCT/US2024/041915, filed on Aug. 12, 2024.
(60) Provisional application No. 63/519,247, filed on Aug. 12, 2023.
(60) Provisional application No. 63/519,256, filed on Aug. 13, 2023.
(60) Provisional application No. 63/519,436, filed on Aug. 14, 2023.
(60) Provisional application No. 63/550,365, filed on Feb. 6, 2024.
(60) Provisional application No. 63/550,379, filed on Feb. 6, 2024.

(51) Int. Cl. A61N 7/00; A61B 18/00; A61B 18/18; A61N 1/05

(52) U.S. Cl. CPC A61N 7/00; A61B 18/1815; A61N 1/0551; A61B 2018/00642

(57) ABSTRACT

Provided are systems, devices, and methods for energy delivery to a patient to treat a medical condition. A system for delivering energy to a patient includes: a treatment device having one or more energy delivery elements. The treatment device can deliver energy, such as ultrasound energy, to a target location of the patient via the one or more energy delivery elements. The energy delivery to the target location treats a medical condition of the patient.

### RELATED APPLICATIONS

This application is a continuation of PCT/US2024/041915, filed on Aug. 12, 2024 and titled, “Brain Modulation Systems and Methods,” which is hereby incorporated by reference in its entirety.

PCT/US2024/041915 claims benefit of U.S. Provisional Patent Application Ser. No. 63/519,247, titled “Device for Multifocal Delivery of Ultrasound into Deep Brain Regions in Humans,” filed Aug. 12, 2023, the content of which is incorporated herein by reference in its entirety for all purposes.

PCT/US2024/041915 claims benefit of U.S. Provisional Patent Application Ser. No. 63/519,256, titled “Remote Targeted Electrical Stimulation,” filed Aug. 13, 2023, the content of which is incorporated herein by reference in its entirety for all purposes.

PCT/US2024/041915 claims benefit of U.S. Provisional Patent Application Ser. No. 63/519,436, titled “Durable Effects of Deep Brain Ultrasonic Neuromodulation on Major Depression,” filed Aug. 14, 2023, the content of which is incorporated herein by reference in its entirety for all purposes.

PCT/US2024/041915 claims benefit of U.S. Provisional Patent Application Ser. No. 63/550,365, titled “Approach and Device for Targeted Neuromodulation Treatment of Chronic Pain,” filed Feb. 6, 2024, the content of which is incorporated herein by reference in its entirety for all purposes.

PCT/US2024/041915 claims benefit of U.S. Provisional Patent Application Ser. No. 63/550,379, titled “Approach and Device for Controlled Targeted Neuromodulation Treatment of Depression,” filed Feb. 6, 2024, the content of which is incorporated herein by reference in its entirety for all purposes.

### FIELD OF THE INVENTIVE CONCEPTS

The present inventive concepts relate generally to systems, devices, and methods for applying and regulating ultrasound to a specific target in the human brain.

### BACKGROUND

Numerous patients exhibiting undesired mental and neurological conditions are treatment-resistant. Current neuromodulation approaches have had limited success. There is a need for improved brain modulation systems and methods.

### SUMMARY

According to an aspect of the present inventive concepts, a system for delivering energy to a patient comprises a treatment device comprising one or more energy delivery elements. The treatment device is configured to deliver energy to a target location of the patient via the one or more energy delivery elements. The energy delivery to the target location treats a medical condition of the patient.

In some embodiments, the target location comprises a location of the brain of the patient.

In some embodiments, the treatment device is configured to deliver energy to an anatomical location of the patient selected from the group consisting of: brain; heart; liver; pancreas; spleen; dorsal root ganglia; spinal cord; peripheral nerves; and combinations thereof.

In some embodiments, the treatment device is configured to deliver ultrasound energy. The treatment device can be configured to further deliver magnetic field energy. The magnetic field can comprise a magnetic field of at least 0.5 T.

In some embodiments, the treatment device is configured to deliver one, two, or more energies selected from the group consisting of: sound energy, such as ultrasound energy; light energy, such as laser light energy; thermal energy, such as heat energy and/or cryogenic energy; electromagnetic energy, such as radiofrequency energy, microwave energy, and/or electroporation energy; chemical energy; mechanical energy; and combinations thereof.

In some embodiments, the target location spans at most 10 mm, and the system is configured to accurately deliver the energy into the target location. The target location can span at most 5 mm.

In some embodiments, the treatment device is configured to deliver the energy with a spatial resolution of no more than 1.0 mm and a temporal accuracy of no more than 5 μs.

In some embodiments, the one or more energy delivery elements comprise multiple energy delivery elements arranged in one or more spherically focused arrays. The one or more spherically focused arrays can each comprise a radius of at least 10 mm, no more than 5,000 mm, or both.

In some embodiments, the treatment device comprises a first treatment assembly configured to deliver energy and to be positioned on the right side of the head of the patient and a second treatment assembly configured to deliver energy and to be positioned over the left side of the head of the patient. The system can further comprise a housing configured to position the first and second treatment assemblies to each deliver the energy through the temporal bone window of the patient's skull.

In some embodiments, the system is configured to deliver energy to multiple different target locations of the deep brain without moving the treatment device and without moving the patient's head. The system can be configured to deliver energy to at least a first target location and a second target location in a sequential arrangement.

In some embodiments, the system comprises a housing and a patient mask that are collectively configured to reproducibly position the patient's head in a desired arrangement for delivery of energy without use of MRI for guidance.

In some embodiments, the system is configured to validate targeting of energy delivery via use of MRI blood-oxygen-dependent imaging. The images can provide target location guidance information, dosimetry information, or both.

In some embodiments, the system further comprises an algorithm configured to determine energy delivery drive signals that compensate for one or more obstacles present in the path of energy delivery from the treatment device to the target location. The one or more obstacles can comprise an obstacle selected from the group consisting of: the skull; the scalp; hair; a component of the system; and combinations thereof. The medical condition treated can comprise depression. The medical condition treated can comprise pain, such as chronic pain. The medical condition treated can comprise addiction, anxiety, and/or other psychological disorder. The medical condition treated can comprise cognitive decline, such as Mild Cognitive Decline. The medical condition treated can comprise Alzheimer's disease.

According to another aspect of the present inventive concepts, a method of treating a patient with a medical condition comprises: selecting a patient for treatment; selecting a system for delivering energy to the patient, the system comprising a treatment device for delivering the energy to a target location of the patient; and delivering energy to the target location of the patient via the treatment device. The delivery of energy treats the medical condition of the patient.

In some embodiments, the energy delivered comprises ultrasound energy.

In some embodiments, the energy delivered further comprises magnetic field energy.

In some embodiments, the medical condition treated comprises pain. The patient selected can have an average 24-hour visual analogue scale pain score of at least 1. The patient selected can have an average 24-hour visual analogue scale pain score of at least 3. The patient selected may have experienced at least 3 months of a moderate level of pain. The target location can comprise the anterior cingulate cortex. The target location can comprise at least two target locations within the anterior cingulate cortex. The target location can comprise at least four target locations within the anterior cingulate cortex. The at least four target locations comprise four locations selected from the group consisting of: subgenual ACC (Brodmann Area 2565) and 6 target locations from within the pACC to aMCC (Brodmann Areas s24, p24, a24, 33). The at least four target locations can? comprise eight target locations within the anterior cingulate cortex. Each target location can be separated from a neighboring target location by at least 2 mm, by no more than 6 mm, or both. Each target location can be separated from a neighboring target location by approximately 4 mm. The energy delivery can comprise a delivery of energy with a peak intensity of no more than 300 W/cm2. The energy delivery can comprise a delivery of energy with a peak intensity of no more than 225 W/cm2, and/or no more than 190 W/cm2. The energy delivery can comprise a delivery of energy of no more than 3 hours, no more than 2 hours, and/or no more than 1 hour. The energy delivery can comprise a delivery of energy for at least 30 minutes, no more than 120 minutes, or both. The energy delivery can comprise a delivery of energy for approximately 40 minutes. The energy delivery can comprise a delivery of energy with field dimensions less than 5 mm by 5 mm by 40 mm. The energy delivery can comprise a delivery of energy with field dimensions of approximately 2.4 mm by 3.6 mm by 20.4 mm. The delivery of energy can comprise multiple test energy deliveries configured to test for symptom reduction. Each test energy delivery can comprise a delivery of energy for no more than 60 seconds. The energy delivery can comprise multiple energy deliveries configured to provide a therapeutic benefit to the medical condition. The multiple energy deliveries can comprise at least four energy deliveries of at least one minute in duration. The multiple energy deliveries can comprise approximately 12 deliveries of energy, each of three minutes in duration. The energy delivery can comprise a delivery of energy with an amplitude of at least 0.5 MPa. The energy delivery can comprise a delivery of energy with an amplitude of approximately 1 MPa. The energy delivery can comprise a delivery of energy comprising bursts of at least 10 msec in duration that can be delivered at less than 90% duty cycle. The bursts can be separated by a burst interval of no more than 20 seconds. The energy delivery can comprise a delivery of energy comprising bursts of approximately: 30 msec in duration; 50% duty cycle; and 0.7 second burst interval. The treatment can achieve an efficacy comprising one or more of: at least a 30% reduction in pain immediately following energy delivery; at least a 21.5% reduction in pain at 1 day after energy delivery; and/or at least a 16.5% reduction in pain 7 days following energy delivery. The treatment can achieve a reduction in absolute VAS pain score of at least one. The treatment can achieve a reduction in absolute VAS pain score of 2.7±1.4. The treatment can have at least a 50% expectation of achieving a 33% reduction in pain immediately following the energy delivery. The treatment can have an approximately 75% expectation of achieving a 33% reduction in pain immediately following the energy delivery. The treatment can have at least a 30% expectation of achieving a 33% reduction in pain 24 hours after the energy delivery. The treatment can have approximately a 60% expectation of achieving a 33% reduction in pain 24 hours after the energy delivery. The treatment can achieve at least a 10% reduction in PROMIS pain intensity score. The treatment can achieve a reduction in the PROMIS pain intensity score of mean±SD reduction of 5.68±7.2 points. The treatment can achieve at least a 10% reduction in PROMIS depression score. The treatment can achieve a reduction in the PROMIS depression score of 2.27±3.75. The treatment can achieve at least a 10% reduction in PROMIS anxiety score. The treatment can achieve a reduction in the PROMIS anxiety score of 2.87±6.21.

In some embodiments, the medical condition treated comprises depression. The patient selected can have treatment resistant depression. The patient selected can have a primary DSM-5 diagnosis of major depressive disorder or bipolar disorder. The patient selected can have, for at least 1 month, a current moderate-to-severe depressive episode without psychotic features. The patient selected can have, for at least 2 months, a current moderate-to-severe depressive episode without psychotic features. The patient selected can have a QIDS score of at least 6. The patient selected can have a QIDS score of at least 10. The target location can comprise a location of the subcallosal cingulate cortex. The target location can comprise at least two target locations within the subcallosal cingulate cortex. The target location can comprise at least three target locations within the subcallosal cingulate cortex. The at least two target locations can comprise midline target locations of the subcallosal cingulate cortex. The at least two target locations can be stimulated sequentially. The at least two target locations can comprise a first target location, a second target location, and a third target location, and the first target location can be at least 2 mm and/or no more than 6 mm anterior to the second target location, and the third target location can be at least 2 mm posterior and/or no more than 6 mm posterior to the second target location. The first target location can be approximately 4 mm anterior to the second target location, and the third target location can be approximately 4 mm posterior to the second target location. The energy delivery can comprise a delivery of energy with a peak intensity of no more than 300 W/cm2. The energy delivery can comprise a delivery of energy with a peak intensity of no more than 225 W/cm2, and/or no more than 190 W/cm2. The energy delivery can comprise a delivery of energy of no more than 3 hours, no more than 2 hours, and/or no more than 1 hour. The energy delivery can comprise a delivery of energy with an amplitude of at least 0.5 MPa. The energy delivery can comprise a delivery of energy with an amplitude of approximately 1 MPa. The energy delivery can comprise a delivery of energy with field dimensions less than 5 mm by 5 mm by 40 mm. The energy delivery can comprise a delivery of energy with field dimensions of approximately 2.4 mm by 3.6 mm by 20.4 mm. The energy delivery can comprise a delivery of energy comprising bursts of at least 10 msec in duration that can be delivered at less than 90% duty cycle. The bursts can be separated by a burst interval of no more than 20 seconds. The energy delivery can comprise a delivery of energy comprising bursts of approximately: 30 msec in duration; 50% duty cycle; and 0.7 second burst interval. The energy delivery can comprise a delivery of energy comprising bursts of approximately: 30 msec in duration; 50% duty cycle; and 1.4 second burst interval. The energy delivery can comprise a delivery of energy for at least 30 seconds. The energy delivery can comprise a delivery of energy for approximately 60 seconds. The energy delivery can comprise a delivery of energy in blocks comprising rest epochs and active sonication epochs. The rest epochs and active epochs can be interleaved. The active epochs and/or the rest epochs can comprise a duration of at least 30 seconds. The active epochs and/or the rest epochs can comprise a duration of approximately 1 minute. The active epochs and/or the rest epochs can comprise a duration of approximately 3 minutes. Each block can comprise at least six total epochs. Each block can comprise approximately 10 total epochs. At least two blocks of energy can be delivered. The energy delivery parameters can vary between blocks. The energy delivery duration can vary between blocks. At least three blocks of energy can be delivered. Each block can comprise at least three test energy deliveries of at least one-minute in duration. Each block can comprise approximately: three to five one-minute sonications with 1.5 second burst intervals, such as to test the tolerability of each target location. Each block can comprise at least six energy deliveries of at least two minutes in duration. Each block can comprise approximately: six three-minute sonications with 1.4 second burst intervals, and/or six three-minute sonications with 0.7 second burst intervals. The treatment can achieve at least a 50% change in sadness. The treatment can achieve approximately a 63% change in sadness. The treatment can achieve a change in HRDS-6 of at least 25% at day 1 after the energy delivery. The treatment can achieve a change in HRDS-6 of approximately 55% at day 1 after the energy delivery. The treatment can achieve a change in HRDS-6 of at least 20% at day 7 after the energy delivery. The treatment can achieve a change in HRDS-6 of approximately 52% at day 7 after the energy delivery.

In some embodiments, the medical condition treated comprises: addiction; anxiety; and/or other psychological disorder. The target location can comprise one, two, or more locations selected from the group consisting of: subcallosal cingulate cortex; nucleus accumbens; cingulate cortex; and combinations thereof. The medical condition treated can comprise cognitive decline. The medical condition treated can comprise mild cognitive decline. The patient selected can have a Montreal Cognitive Assessment score of no more than 25. The target location can comprise one, two, or more locations selected from the group consisting of: hippocampus; amygdala; entorhinal cortex; cingulate cortex; fornix and combinations thereof. The medical condition treated can comprise Alzheimer's disease. The patient selected can have a Montreal Cognitive Assessment score of no more than 15. The target location can comprise one, two, or more locations selected from the group consisting of: hippocampus; amygdala; entorhinal cortex; cingulate cortex; fornix; and combinations thereof.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The content of all publications, patents, and patent applications mentioned in this specification are herein incorporated by reference in their entirety for all purposes.

### BRIEF DESCRIPTION OF THE DRAWINGS

FIG. 1 illustrates a block diagram of an embodiment of a system for performing a medical procedure on a patient, consistent with the present inventive concepts.

FIG. 1A illustrates a block diagram of another embodiment of a system for performing a medical procedure on a patient, consistent with the present inventive concepts.

FIG. 1B illustrates a block diagram of another embodiment of a system for performing a medical procedure on a patient, consistent with the present inventive concepts.

FIGS. 2A-B illustrate an anatomical schematic and a photograph, respectively, of a portion of a system for delivering energy to a brain of a patient, consistent with the present inventive concepts

FIGS. 2C-D illustrate coronal and sagittal views, respectively, of a patient-specific brain anatomy, consistent with the present inventive concepts.

FIG. 3A illustrates an example pressure field measured in three dimensions through an ex-vivo human skull, consistent with the present inventive concepts.

FIG. 3B illustrates an electronic targeting range measured through four ex-vivo skulls, consistent with the present inventive concepts.

FIGS. 4A-B are bar charts related to the relative distance of energy-delivery elements and markers for five patients, consistent with the present inventive concepts.

FIG. 5 includes two bar charts illustrating a positive effect of SCC modulation, consistent with the present inventive concepts.

FIGS. 6A-C are a brain image, a graph, and another brain image, respectively, related to a study S2, consistent with the present inventive concepts.

FIG. 7 is a graph demonstrating change in Hamilton Depression Rating Score that results from ultrasound treatment, consistent with the present inventive concepts.

FIG. 8 is a table of adverse effects encountered in study S2, consistent with the present inventive concepts.

FIGS. 9A-B are a vector presentation and a graph, respectively, representing electric field properties, consistent with the present inventive concepts.

FIG. 10A-D are a first vector presentation, a second vector presentation, a first pair of graphs, and a second pair of graphs, related to electric field measurement and properties, consistent with the present inventive concepts.

FIGS. 11A-C are a first bar chart, a graph, and a second bar chart, respectively, related to response magnitude for the combination of an ultrasound delivery and a magnetic field delivery, consistent with the present inventive concepts.

FIGS. 12A-C are an anatomical schematic, two brain thermometry images, and a graph of gamma activity, respectively, consistent with the present inventive concepts.

FIGS. 13A-C are a flow chart, an anatomical schematic, and two brain images, respectively, consistent with the present inventive concepts.

FIG. 14 is a bar chart illustrating pain intensity change percentage for sham and active stimulation of the ACC, consistent with the present inventive concepts.

FIG. 15 is a graph of pain intensity change over time for sham and active stimulation of the ACC, consistent with the present inventive concepts.

FIGS. 16A-B are two bar charts representing response rates comparing active and sham stimulations, consistent with the present inventive concepts.

FIG. 17 is a bar chart representing PROMIS Pain Intensity change comparing sham and active stimulations, consistent with the present inventive concepts.

FIGS. 18A-B are MRI images of the subgenual cingulate cortex and dorsal anterior cingulate cortex, respectively, of patients in a study S4, consistent with the present inventive concepts.

FIGS. 19A-C are a first brain image, a second brain image, and a graph of target modulation, respectively, from a study S5, consistent with the present inventive concepts.

FIG. 20 shows 3 MRI images of a patient's brain illustrating selective deactivation of the SCC, consistent with the present inventive concepts.

FIG. 21 is a bar chart illustrating Sadness change in sham and active stimulation, consistent with the present inventive concepts.

FIGS. 22A-C are a first graph showing HDRS-6 score change, a second graph showing percent HDRS-6 score change, and a bar chart showing response rate, respectively, for sham and active stimulation, consistent with the present inventive concepts.

FIG. 23 is a flow chart representing the study design of study S5, consistent with the present inventive concepts.

FIG. 24 is a set of brain images showing individual SCC ultrasound targets, consistent with the present inventive concepts.

FIG. 25 is a consort diagraph of study participants of study S5, consistent with the present inventive concepts.

FIG. 26 is a set of brain images representing distributed neuromodulation during SCC stimulation, consistent with the present inventive concepts.

FIG. 27 is a table representing score changes for the expanded Positive and Negative Affect Schedule of study S5, consistent with the present inventive concepts.

FIGS. 28A-C are bar charts representing Sadness change, HDRS-6 score change, and percentage HDRS-6 change, respectively, for sham and active stimulation, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element (also referred to as a “component” herein) is described as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

As used herein, the terms “operably attached”, “operably connected”, “operatively coupled” and similar terms related to attachment of components shall refer to attachment of two or more components that results in one, two, or more of: electrical attachment; fluid attachment; magnetic attachment; mechanical attachment; optical attachment; sonic attachment; and/or other operable attachment arrangements. The operable attachment of two or more components can facilitate the transmission between the two or more components of: power; signals; electrical energy; fluids or other flowable materials; magnetism; mechanical linkages; light; sound such as ultrasound; and/or other materials and/or components.

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a blood or other fluid delivery location), shall include components positioned near to the anatomical site, as well as components positioned in, on and/or within the anatomical site.

Spatially relative terms, such as “beneath,” “below,” “lower,” “above,” “upper”, “under” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention”, as well as “avoid” and “avoiding”, shall include the acts of “reduce”, “reducing”, and “reduction”, respectively.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

As used herein, when a quantifiable parameter is described as having a value “between” a first value X and a second value Y, it shall include the parameter having a value of: at least X, no more than Y, and/or at least X and no more than Y. For example, a length of between 1 and 10 shall include a length of at least 1 (including values greater than 10), a length of less than 10 (including values less than 1), and/or values greater than 1 and less than 10.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the terms “about” or “approximately” shall refer to ±20% of a stated value.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g. efficacious therapy) and/or to prevent and/or otherwise reduce (hereinafter “prevent”) an undesired event (e.g. a device and/or clinical adverse event). In some embodiments, a threshold value is determined to include a safety margin, such as to account for patient, user, and/or operator variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

As used herein, the term “functional element” is to be taken to include one or more elements constructed and arranged to perform a function. A functional element can comprise a sensor and/or a transducer. In some embodiments, a functional element is configured to deliver energy. In some embodiments, a functional element is configured to treat tissue (e.g. a functional element configured as a treatment element). Alternatively or additionally, a functional element (e.g. a functional element comprising a sensor) can be configured to record one or more parameters, such as a patient physiologic parameter; a patient anatomical parameter (e.g. a tissue geometry parameter); a patient environment parameter; and/or a system parameter. In some embodiments, a sensor or other functional element is configured to perform a diagnostic function (e.g. to gather data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record and/or otherwise sense a parameter such as a patient physiologic parameter or a system parameter; and combinations of one or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise an expandable assembly. A functional assembly can comprise one or more functional elements.

The term “transducer” where used herein is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as: light (e.g. a transducer comprising a light emitting diode or light bulb), sound (e.g. a transducer comprising a piezo crystal configured to deliver ultrasound energy); pressure (e.g. an applied pressure or force); heat energy; cryogenic energy; chemical energy; mechanical energy (e.g. a transducer comprising a motor or a solenoid); magnetic energy; and/or a different electrical signal (e.g. different than the input signal to the transducer). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); ultrasound and/or other sound energy to tissue (e.g. a transducer comprising a piezo crystal); chemical energy; electromagnetic energy; magnetic energy; and combinations of one or more of these.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

As used herein, the term “user interface” can comprise one or more interfaces, each interface comprising one or more components configured to receive an input from a user, “user input device” herein, and/or one or more components configured to provide output to a user, “user output device” herein. An input device can comprise one, two, three, or more components selected from the group consisting of: keyboard; a mouse; a button; a switch; a lever; a keypad such as a membrane keypad; a joystick; a touchscreen display; a microphone; a brain-machine-interface (e.g., a thought-control device); a camera, such as a camera with eye tracking, motion tracking, gesture identification, and/or other image processing capability configured to identify user input; a motion capture device, such as a camera and/or a device including one or more accelerometers; a virtual input device, such as a virtual device comprising ultrasonic, image capture, and/or motion-based sensing of user inputs; a physiologic input sensor, such as a sensor configured to provide an input signal based on a user action, such as flexure of a muscle proximate the sensor; a scent detector, such as a detector configured to identify a pheromone or other scent produced by the user; other input component; and combinations of these. An output device can comprise one, two, three, or more components selected from the group consisting of: a visual output component such as a light and/or a display such as a touchscreen display; an audible output component such as a buzzer and/or a speaker; a haptic output component such as a vibrational transducer and/or an ultrasonic device configured to produce a tactile output; a brain-machine-interface; an augmented reality (AR) and/or a virtual reality (VR) output device, such as glasses or a headset including a non-transparent display, a transparent display, and/or a “heads up” display where information is presented to the user in an overlay manner; a scent output device configured to produce an aromatic output, such as a computerized scent output; other output component; and combinations of these.

The terms “data” and “information” are used interchangeably herein.

As used herein, the term “access” can refer to providing access to a location within a patient for delivery of fluids or other materials, and/or removal of fluids or other materials.

As used herein, “therapy planning”, “therapy plan”, and the like, can comprise a set of one or more medical procedures (e.g., diagnostic and/or therapeutic procedures) to be performed using the systems, devices, and methods of the present inventive concepts. A therapy plan can include: the anatomical locations of one or more portions of tissue to be treated, and/or one or more anatomical locations of one or more portions of tissue to which treatment should be avoided; the settings of energy delivery (e.g., ultrasound delivery) to be used in a diagnostic procedure (e.g., an imaging procedure or other diagnostic procedure); the settings of energy delivery (e.g., ultrasound delivery) to be used in a therapeutic procedure (e.g., an ablation procedure, stimulation procedure, and/or other therapeutic procedure); the identity of one or more clinicians to perform a medical procedure; and combinations of these.

As used herein, “acoustic pathway” can refer to the paths in volumes of tissue through which energy (e.g., ultrasound energy) travels.

It is appreciated that certain features of the inventive concepts, which are, for clarity, described in the context of separate embodiments, may also be provided in combination in a single embodiment. Conversely, various features of the inventive concepts which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the inventive concepts have been simplified to focus on elements that are relevant for a clear understanding of the inventive concepts, while eliminating, for purposes of clarity, other elements that those of ordinary skill in the art will appreciate may also comprise a portion of the inventive concepts. However, because such elements are well known in the art, and because they do not necessarily facilitate a better understanding of the inventive concepts, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

Provided herein are systems, devices, and methods for performing a medical procedure (also referred to as a “clinical procedure” herein) on a patient, such as a diagnostic procedure, a therapeutic procedure (also referred to as a “treatment procedure” herein), and/or other medical procedure. In some embodiments, at least ultrasound energy is delivered to a patient (e.g., through the skull of the patient) in order to treat a medical condition of a patient. In some embodiments, ultrasound energy and magnetic energy are delivered (e.g., simultaneously) to a patient (e.g., through the skull of the patient) in order to treat a medical condition of a patient. Typical medical conditions treated by the systems, devices, and methods of the present inventive concepts include, but are not limited to: pain; depression, anxiety; addiction, and/or other psychological disorder, cognitive impairment such as mild cognitive impairment; Alzheimer's disease; other medical condition; and combinations of one, two, or more of these.

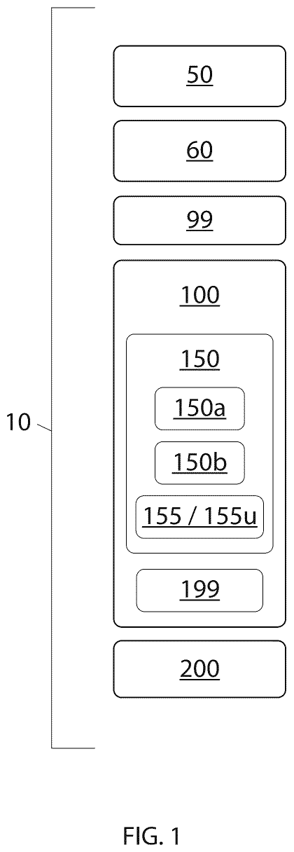

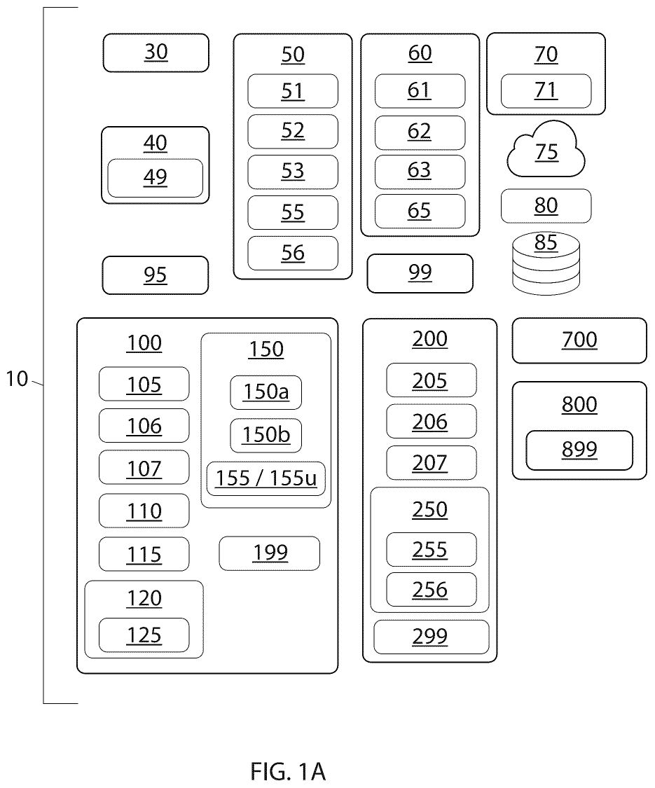

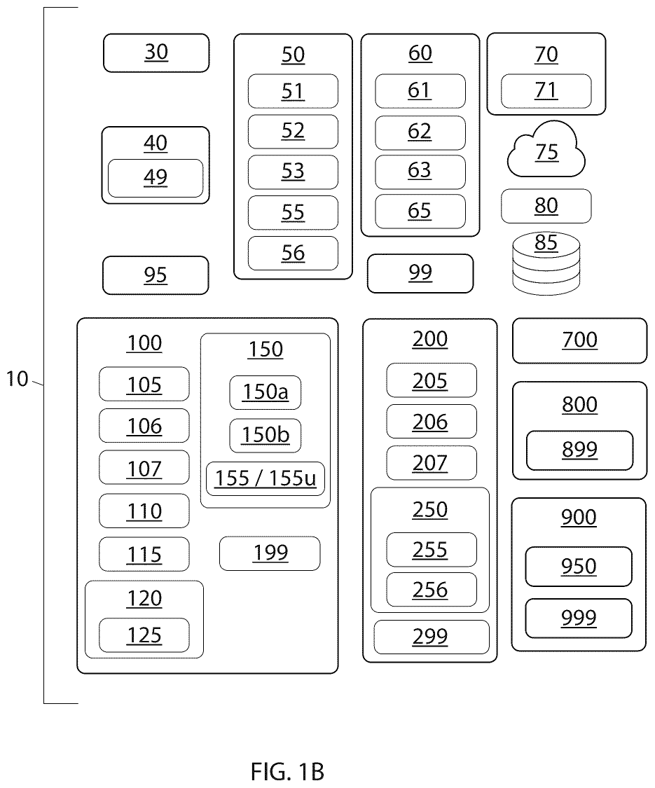

Referring now to FIG. 1, a system for performing a medical procedure on a patient is illustrated. System 10 can be configured and/or adapted (“configured” or “adapted” herein) to perform a medical procedure on a patient comprising a diagnostic procedure, a treatment procedure, or both. The patient can comprise a human and/or other mammalian patient, “patient” herein. System 10 can be configured to modulate, stimulate, and/or otherwise treat one or more brain locations, such as when configured to modulate, stimulate, and/or otherwise treat a deep brain circuit of a patient. Alternatively, or additionally, system 10 can be configured to produce image and/or other diagnostic data related to one or more brain locations, such as when configured to produce diagnostic data related to a deep brain circuit of a patient.

System 10 comprises one, two, or more treatment devices, treatment device 100 as shown. Treatment device 100 can comprise a device selected from the group consisting of: a device configured to be mounted (e.g., fixedly and/or removably mounted) to a table-top or other surface; a head-worn device; a device attached to a manipulatable arm; and combinations of these. As used herein, a “user”, “operator”, and/or “clinician” of system 10 can refer to a doctor, nurse, clinician and/or other healthcare professional, that uses treatment device 100 and/or other component of system 10. System 10, treatment device 100, and/or other components of system 10 of FIG. 1 can be of similar construction and arrangement as the similar components described in reference to FIG. 1A and/or otherwise herein.

Treatment device 100 can comprise one, two, or more assemblies for treating tissue, treatment assembly 150 shown. Each treatment assembly 150 can comprise, one, two, or more energy delivery transducers, energy delivery elements 155 shown (e.g., one, two, or more ultrasound transducers). Treatment assembly 150 can be configured to deliver one, two, or more forms of energy, as described herein. In some embodiments, treatment assembly 150 is configured to perform a diagnosis (e.g., in addition to a treatment or instead of a treatment), such as a diagnosis of a medical condition of a patient, a diagnosis (e.g., an assessment) of a procedure being performed by system 10, and/or other diagnosis. In some embodiments, treatment assembly 150 comprises an assembly of one or more ultrasound transducers such as when treatment assembly 150 is configured to deliver ultrasound energy to tissue, such as to modulate, stimulate, and/or otherwise treat tissue (“modulate”, “stimulate” or “treat” herein), and/or to deliver ultrasound energy to create image data and/or other diagnostic data (e.g., when treatment assembly 150 is configured to both deliver ultrasound energy and receive ultrasound energy). Treatment assembly 150 can comprise an assembly including one or more arrays of ultrasound transducers, where each array can comprise one or more ultrasound transducers. In some embodiments, treatment assembly 150 comprises a first portion, treatment assembly 150a and a second portion, treatment assembly 150b, each as shown. In some embodiments, during a medical procedure performed using system 10, treatment assembly 150a is positioned on one side of a patient's head, and treatment assembly 150b is positioned on a relatively opposite side of the patient's head, as described herein. Treatment assembly 150 can be of similar construction and arrangement to assembly 150 described in reference to FIG. 1A and/or otherwise herein.

System 10 can comprise one, two, or more consoles and/or other modular assemblies, console 200 shown. Console 200 can be configured to operably connect to treatment device 100, and/or another component of system 10, such as when the attachment comprises an electrical attachment (e.g., to transfer power, data, and/or other signals), a fluid attachment (e.g. to transfer cooling fluid, hydraulic fluid, pneumatic fluid, and/or other fluid), an optical attachment (e.g. to transfer laser light and/or other light); a mechanical attachment (e.g. to operate a mechanical linkage); an acoustic attachment (e.g., to transfer sound), and/or other attachment. Console 200 can be configured to operably connect to treatment device 100, and/or another system 10 component, for example to transmit power and/or signals to, and/or receive signals from the connected component. Console 200 can provide a user interface (e.g., as described herein) for the input of commands and/or other information from a user of system 10, and/or for the output of information from system 10 to a user. Console 200 can comprise one or more discrete components. Console 200 can comprise a cart or other self-standing component, and/or a component intended to be placed on a desk, table, or other above the floor surface. Console 200 can comprise one, two, or more components selected from the group consisting of: a laptop, tablet, and/or other computer. In some embodiments, all or a portion of console 200 comprise a hand-held device. In some embodiments, treatment device 100 comprises all or at least a portion of console 200. Console 200 can be of similar construction and arrangement as console 200 described in reference to FIG. 1A and/or otherwise herein.

System 10 can comprise one, two, or more modules configured to perform a function, processing unit 50 shown. Treatment device 100, console 200, and/or another component of system 10 can comprise all or a portion of a processing unit 50. Processing unit 50 can be of similar construction and arrangement as processing unit 50 described in reference to FIG. 1A and/or otherwise herein.

System 10 can comprise one, two, or more modules configured to provide a user interface, user interface 60 shown. Treatment device 100, console 200, and/or another component of system 10 can comprise all or a portion of a user interface 60. User interface 60 can be of similar construction and arrangement as user interface 60 described in reference to FIG. 1A and/or otherwise herein.

System 10 (e.g., any component of system 10) can comprise one, two, or more elements, assemblies, and/or other components configured to perform a function, functional element 99 shown. Treatment device 100, console 200, and/or another component of system 10 can comprise all or a portion of one or more functional elements 99. Functional element 99 can be of similar construction and arrangement as functional element 99 described in reference to FIG. 1A and/or otherwise herein.

In some embodiments, system 10 is configured to perform an imaging procedure and/or otherwise collect image data, image data ID, such as when one or more ultrasound transducers (e.g., energy delivery elements 155 comprising ultrasound transducers, USTs 155U herein) of treatment assembly 150 deliver ultrasound to tissue, and one or more ultrasound transducers (e.g., similar and/or dissimilar transducers of energy delivery elements 155) of treatment assembly 150 receive the delivered ultrasound (e.g., receive reflections of the delivered ultrasound), such that system 10 can produce one or more images of tissue (e.g., target tissue as described herein) based on the timing and/or other parameters of the delivered and received ultrasound. System 10 can be configured to produce two-dimensional (2D) and/or three-dimensional (3D) images based on the collected image data ID.

In alternative and/or additional embodiments, system 10 is configured to perform a “treatment procedure”, such as a treatment procedure including the delivery of ultrasound energy. System 10 can be configured to perform a treatment procedure comprising a tissue treatment procedure, such as a tissue modulation and/or other tissue treatment procedure including the delivery of energy (e.g., ultrasound energy, radiofrequency energy, magnetic energy, and/or one or more other forms of energy as described herein) to tissue. In some embodiments, delivered energy can comprise ultrasound energy that is delivered to tissue by one or more transducers of energy delivery elements 155, such as ultrasound energy delivered to “target tissue” (also referred to as a “target location”) to be modulated and/or otherwise treated. In some embodiments, system 10 is configured to deliver ultrasound energy to activate a pharmaceutical and/or other agent; and/or to enhance the efficacy of a pharmaceutical and/or other agent.

In some embodiments, system 10 is configured to perform a “combined imaging and treatment procedure” which comprises the performance of one or more imaging procedures, as well as one or more tissue modulation and/or other tissue treatment procedures. In these embodiments, system 10 can be configured to simultaneously produce image data ID (e.g., such as to create images of tissue, an agent, and/or an implant within the patient), and deliver a treatment (e.g., a treatment comprising modulating and/or otherwise treating tissue, and/or a treatment modifying an implant and/or agent). Alternatively, or additionally, system 10 can be configured to sequentially (e.g., in a repeating manner) create image data ID (e.g., such as to create images of tissue, an agent, and/or an implant within the patient), and deliver a treatment (e.g., a treatment comprising modulating and/or otherwise treating tissue, and/or a treatment modifying an implant and/or agent). In the combined imaging and treatment procedures, an energy delivery and/or other parameters of a treatment procedure (e.g., a tissue treatment procedure) can be determined (e.g., automatically determined by system 10) based on image data ID collected simultaneously with, and/or prior to the performance of the treatment.

In some embodiments, system 10 is configured to perform: imaging of a brain (e.g., imaging of one or more portions of a brain), such as to identify the location of one or more target tissue locations to be treated, such as to perform a modulation of one or more deep brain circuits, as described herein. In some embodiments, location of one or more target locations to be treated (e.g., to receive ultrasound and/or other form of energy) is based on data collected during a procedure in which the patient's brain is imaged. The brain imaging procedure can be performed using ultrasound energy (e.g., as delivered and received by treatment device 100), using an MRI (e.g., via an imaging device 800 comprising an MRI), or both.

In some embodiments, system 10 is configured to perform: imaging (e.g., used to perform a diagnostic analysis such as a diagnosis to determine locations of target tissue to be treated); treatment (e.g., modulation and/or other tissue treatment); or both, such as when one or both procedures are performed at an anatomical location selected from the group consisting of: brain; heart; liver; pancreas; spleen; dorsal root ganglia; spinal cord; peripheral nerves; and combinations of these.

In some embodiments, system 10 is configured to perform: imaging (e.g., used to perform a diagnostic analysis, such as a diagnosis to determine locations of target tissue to be treated); treatment (e.g., modulation and/or other tissue treatment); or both, such as when one or both procedures are performed to diagnose and/or treat a tissue type selected from the group consisting of: brain tissue; heart tissue; liver tissue; pancreas tissue; spleen tissue; dorsal root ganglia tissue; spinal cord tissue; peripheral nerve tissue; and combinations of these.

System 10 can be configured to avoid affecting certain volumes of tissue, “non-target tissue”, also referred to as a “non-target location” herein. In some embodiments, system 10 is configured to perform a diagnostic procedure in which damage and/or other undesired effect upon non-target tissue is prevented or at least reduced (“reduced”, “prevented” or “avoided” herein). In some embodiments, tissue immediately proximate target tissue comprises “safety margin tissue” (also referred to as “safety margin location” herein), and tissue beyond the safety margin tissue comprises non-target tissue. In these embodiments, safety margin tissue can comprise tissue to which treatment (e.g., modulation or other treatment) is not particularly desired but not necessary to avoid.

As described herein, system 10 can be configured to perform both a diagnostic procedure (e.g., a procedure including the production of one or more images of tissue or other material on and/or within the patient), as well as a treatment procedure (e.g., a procedure in which target tissue is modulated and/or otherwise treated). In these embodiments, the diagnostic procedure (e.g., imaging) and treatment procedure can be performed simultaneously, sequentially, or both. In some embodiments, imaging and tissue treatment (e.g., modulation) are performed in an alternating arrangement, such as when tissue treatment is adjusted (e.g., one or more ultrasound delivery and/or other treatment parameters are adjusted) based on an analysis of one or more images (also referred to as “image data ID” herein). Adjustment of treatment parameters can be performed by system 10 (e.g., via an algorithm of system 10 as described herein), by a clinician (e.g., based on information provided by system 10), or via a combination of system 10 and a clinician (e.g., when a clinician is required to confirm the acceptability of a parameter or parameter change “suggested” by system 10).

In some embodiments, system 10 is configured to perform a diagnostic and/or treatment procedure comprising the delivery of ultrasound energy to activate a pharmaceutical and/or other agent; and/or to enhance the efficacy of a pharmaceutical and/or other agent.

In some embodiments, system 10 is configured to perform a medical procedure comprising the delivery of ultrasound energy to an implant and/or an agent, such as to supply power to and/or otherwise modify the implant and/or agent.

Target tissue treated using system 10 can comprise two, three, four, or more volumes of target tissue. Each volume of target tissue can be adjacent to another volume of target tissue, and any pair of volumes of target tissue can be non-adjacent. In some embodiments, two or more volumes of target tissue can comprise two or more volumes of tissue of the brain.

In some embodiments, treatment assembly 150 comprises a first portion, assembly 150a that is configured to be positioned on one side of a head of a patient, and a second portion, assembly 150b, that is configured to be positioned on the opposite side of the patient's head, such as is described herein.

In some embodiments, system 10 and/or one or more of its components, are of similar construction and arrangement as the systems and components described in United States Patent Application US20230210493A1, Ser. No. 18/093,220, titled “System and Methods for Modulation of Deep Brain Circuits”, filed Jan. 4, 2023, and/or International PCT application WO2023211898, Serial Number PCT/US2023/019759, titled “System and Method for Sharpening the Focal Volume of Therapeutic and Imaging Systems”, filed Apr. 25, 2023, the contents of each of which is incorporated herein by reference for all purposes.

Referring additionally to FIG. 1A, another embodiment of a system for performing a medical procedure is illustrated, consistent with the present inventive concepts. System 10 of FIG. 1A can be of similar construction and arrangement as system 10 of FIG. 1 and/or otherwise described herein. For example, system 10 of FIG. 1A can include treatment device 100, treatment assembly 150, energy delivery module 250, each as shown, as well as processing unit 50, user interface 60, functional element 99, and console 200, also as shown.

In some embodiments, system 10 further comprises one or more additional imaging devices, imaging device 800 shown where each device is configured to produce image data ID. Treatment device 100 can comprise all or a portion of imaging device 800. Imaging device 800 can comprise one, two, or more devices selected from the group consisting of: fluoroscope or other imaging device; CT scanner; magnetic resonance imager (MRI); positive emission tomography (PET) scanner; ultrasound imager; optical coherence tomography (OCT) and/or other light-based imaging device; and combinations thereof. Imaging device 800 can comprise an MRI configured to identify target tissue for treatment (e.g., to identify one or more volumes of tissue of the brain for modulation and/or other treatment).

As described herein, treatment assembly 150, imaging device 800, and/or one or more other components of system 10 can be configured to produce (also referred to as “record”, “gather”, “collect” and the like) image data, image data ID herein.

Processing unit 50 can comprise one or more modules, where each module can be configured to perform, control, and/or monitor one or more of the functions of system 10 (e.g., as described herein). One or more devices or other components of system 10 can comprise all or a portion of a processing unit 50, such as when all or a portion of a processing unit 50 is integral to: treatment device 100, console 200, imaging device 800, and/or another component of system 10. For example, processing unit 50 can be configured to perform and/or facilitate one or more processes, data collections, data analyses, data transfers, signal processing functions, agent deliveries, positioning of access elements, flow monitoring, monitoring of one or more patient parameters, and/or other functions of system 10 (“functions of system 10”, “system 10 functions” or simply “system functions” herein). Processing unit 50 can comprise one or more electronic elements, electronic assemblies, and/or other electronic components, such as components selected from the group consisting of: microprocessors; microcontrollers; state machines; memory storage components; analog-to-digital converters; rectification circuitry; filters and other signal conditioners; sensor interface circuitry; transducer interface circuitry; and combinations of one, two, or more of these. For example, processing unit 50 can include at least one processor and at least one memory storage component, such as processor 51 and memory 52, each shown. Memory 52 can comprise local memory, an external storage device, and/or a remote or cloud-based memory system. Memory 52 can be coupled to processor 51, and memory 52 can store one or more sets of computer instructions, instructions 53 shown. Instructions 53 can comprise executable instructions that are used to perform an energy delivery compensation (e.g., as described herein), as well as other functions of system 10. Instructions 53 can comprise instructions used by processor 51 to perform one or more algorithms of system 10. For example, system 10 can comprise one or more algorithms, algorithm 55 shown, that are performed by processor 51. Additionally, or alternatively, instructions 53 can comprise instructions for running one or more applications of system 10, for example application 56 shown. Processing unit 50 can be configured to “run” application 56, such that application 56 can initiate, modify, stop, and/or otherwise control the performance of various functions of treatment device 100 and/or of another component system 10. In some embodiments, application 56 is configured to receive input from a user of system 10, for example via a user interface (e.g., user interface 60 described herein). In some embodiments, algorithm 55 can comprise one or more machine learning, neural net, and/or other artificial intelligence algorithms (“AI algorithm” herein). As described herein, all or a portion of one or more processing units 50 can be integrated into one, two, or more of the various components of system 10, such as treatment device 100, console 200, a server (e.g., server 80 described herein), and/or other component of system 10. Performance of a function of system 10 is described hereabove as being performed by processing unit 50. Alternatively, or additionally, the performance of a function of system 10 can be described herein, interchangeably, as being performed by algorithm 55 and/or system 10. For example, “algorithm 55 being configured to perform an action, a routine, and/or another function” can be interpreted as processing unit 50 and/or system 10 being configured to perform the action, routine, and/or other function, and vice versa.

As described herein, processing unit 50 (e.g., processing unit 105 of treatment device 100, processing unit 205 of console 200, and/or a processing unit of another system 10 component), can be configured to perform one or more algorithms, algorithm 55. Each algorithm 55 can comprise an artificial intelligence algorithm or other algorithm. In some embodiments, system 10 is configured to produce a volumetric multi-dimensional image of tissue (e.g., as described herein), and algorithm 55 comprises an artificial intelligence algorithm or other algorithm that is configured to: characterize one or more tissue types; identify tissue areas to avoid treating; and/or suggest tissue areas to be treated.

Algorithm 55 can comprise an artificial intelligence algorithm or other algorithm that is configured to assess image data ID to maintain a focal spot (e.g., a particular field of view) for delivery of energy (e.g., delivery of ultrasound energy to modulate tissue).

User interface 60 can comprise one or more user interfaces configured to provide and/or receive information to and/or from, respectively, a user of the system (e.g., a clinician and/or other user of system 10). One or more devices or other components of system 10 can comprise all or a portion of a user interface 60, such as when all or a portion of a user interface 60 is integral to: treatment device 100, console 200, imaging device 800, and/or another component of system 10. User interface 60 can include one or more user input components and/or output components, as described herein. For example, user interface 60 can comprise a keyboard, mouse, touchscreen, and/or other human interface and/or other input component, user input device 61. In some embodiments, user interface 60 comprises a speaker, indicator light, haptic transducer and/or other human interface and/or other output component, user output device 62. In some embodiments, user output device 62 comprises a video output component, such as display 63 shown. Display 63 can comprise a touchscreen display, for example when user input device 61 and user output device 62 collectively comprise display 63. In some embodiments, processing unit 50 is configured to provide an interactive graphical interface, GUI 65, such as a graphical user interface provided by application 56. GUI 65 can be displayed (e.g., displayed to a user of system 10) via display 63. In some embodiments, user interface 60 and/or GUI 65 comprise a virtual reality and/or augmented reality interface. One or more components of system 10 can comprise one or more portions of a user interface 60, such as treatment device 100, console 200, and/or other components of system 10 described herein.

Communication module 70 can comprise one or more communication modules configured to transmit and/or receive data. One or more devices or other components of system 10 can comprise all or a portion of a communication module 70, such as when all or a portion of a communication module 70 is integral to: treatment device 100, console 200, and/or another component of system 10. Communication module 70 can be configured to provide communication between (e.g., transfer commands, delivery information, patient information, and/or other data between) two or more components of system 10, such as via wired and/or wireless communication. For example, communication module 70 can include one or more transmitters and/or receivers, transceiver 71 shown. Transceiver 71 can comprise a wireless transceiver, such as a Bluetooth transceiver, a Near Field Communication (NFC) transceiver, a Wi-Fi transceiver, a cellular transceiver, a satellite-connected transceiver, and/or other short-range and/or long-range wireless transceiver. A wireless connection can include a short-range wireless connection, such as an NFC connection and/or a Bluetooth low energy (BLE) connection. In some embodiments, communication module 70 is configured to transfer data via an acoustic signal, such as an acoustic signal that is outside of the auditory range of the user. In some embodiments, communication module 70 is configured to communicate via one or more wired and/or wireless networks, such as network 75 shown. Network 75 can include a wireless network, such as a cellular network, LAN, WAN, VPN, the Internet, and/or other wireless network connecting two or more devices. In some embodiments, network 75 comprises a wired network, and/or a network including wired and wireless devices.

Communication module 70 can be configured to transfer data between at least a first component of system 10 and at least a second component of system 10, as described herein. In some embodiments, the first component of system 10 comprises treatment device 100. The second component can comprise another component of system 10, for example console 200, and/or imaging device 800.

Treatment device 100 can comprise one or more housings, housing 110 shown. In some embodiments, housing 110 is configured to position and orient a first treatment assembly 150a relative to a second treatment assembly 150b. In some embodiments, the position, the orientation, or both, is adjustable (e.g., automatically by the system, manually by an operator, or both). In some embodiments, housing 110 surrounds all or a portion of treatment assembly 150.

Treatment device 100 can include one or more markers, marker 115 shown. Marker 115 can comprise one or more markers of device 100 that are used during a registration procedure (e.g., as described herein) and/or a subsequent treatment procedure using registration information to position device 100. Marker 115 can comprise one, two, or more markers (e.g., fiducial markers) selected from the group consisting of: MRI visualizable marker; radiographic marker; ultrasonically visible marker; electrically identifiable marker; electromagnetic marker; visible marker; and combinations of these. Housing 110 can comprise one or more markers 115. Treatment assembly 150 can comprise one or more markers 115.

System 10 can include one or more markers, marker 95 shown, which are configured to be placed on the patient, such as markers on the patient that are used during a registration procedure (e.g., as described herein) and/or a subsequent treatment procedure using registration information to position treatment device 100, the patient, and/or another component of system 10. Marker 95 can comprise one, two, or more markers (e.g., fiducial markers) selected from the group consisting of: MRI visualizable marker; radiographic marker; ultrasonically visible marker; electrically identifiable marker; electromagnetic marker; visible marker; and combinations of these.

In some embodiments, treatment device 100 comprises a user interface 106 comprising at least a portion of user interface 60 of system 10. User interface 106 of treatment device 100 can be positioned on housing 110. User interface 106 can comprise an alert element (e.g., alert element 49 of alert assembly 40 positioned on and/or within housing 110).

In some embodiments, treatment device 100 includes at least a portion of processing unit 50, at least a portion of user interface 60, and/or at least a portion of communication module 70, such as when treatment device 100 comprises processing unit 105, user interface 106, and/or communication module 107, respectively, each shown.

In some embodiments, treatment device 100 comprises one or more modules for sensing, sensor module 120, which can include one or more sensors, sensor 125, each as shown. In some embodiments, sensor 125 comprises one or more thermocouples and/or other temperature sensors, such as temperature sensors used to monitor temperature of one or more portions of treatment assembly 150 and/or another portion of treatment device 100. Sensor 125 can comprise one, two, three, or more sensors selected from the group consisting of: temperature sensor; pressure sensor; strain gauge; accelerometer; physiologic sensor; GPS sensor; and combinations thereof.

Treatment device 100 includes treatment assembly 150 which includes energy delivery elements 155. Energy delivery elements 155 can comprise one or more portions, each portion comprising one, two, or more ultrasound transducers USTs 155U, such as: one, two or more piezo-based ultrasound transducers (e.g., piezoelectric micromachined ultrasonic transducers, PMUTs); one, two or more capacitive micromachined ultrasonic transducers (CMUTs); and/or one, two, or more other form of ultrasound transducer.

Energy delivery elements 155 can comprise one, two, or more arrays of energy delivery transducers (e.g., one, two, or more arrays of ultrasound transducers configured as a phased array configured to allow electronic focusing of ultrasound energy delivery).

In some embodiments, a set of one or more energy delivery transducers of energy delivery elements 155 (e.g., ultrasound-based energy delivery transducers) can be configured to switch between an “imaging mode” and a “treatment mode”, such as when switching between an imaging mode in which imaging-level ultrasound energy is transmitted to tissue and reflections are received from tissue, and a treatment mode in which tissue modulation and/or other treatment-level ultrasound energy is delivered to modulate and/or otherwise treat tissue. Treatment device 100, console 200, and/or another component of system 10 can comprise a switching assembly that is configured to perform the switching of the transducers of energy delivery elements 155 between the imaging mode and the treatment mode.

Console 200 can comprise one or more consoles configured to operably attach to treatment device 100, imaging device 800, and/or one or more other components of system 10. In some embodiments, console 200 includes at least a portion of processing unit 50, at least a portion of user interface 60, and/or at least a portion of communication module 70, such as when console 200 comprises processing unit 205, user interface 206, and/or communication module 207, respectively, each shown.

Console 200 can operably connect to treatment device 100, imaging device 800, and/or another component of system 10 via a wired and/or a wireless connection, such as via a connection provided between communication module 107 of treatment device 100 and communication module 207 of console 200. Console 200 can be configured to receive data, such as data 85 (e.g., to “upload” data 85) from treatment device 100, from user interface 206, from communication module 207, from imaging device 800, and/or from another component of system 10. In some embodiments, console 200 is configured to adjust one or more parameters of the operation of treatment device 100 and/or imaging device 800, for example, based on the analysis of data 85.

Console 200 can comprise one or more consoles, such as one or more similar and/or different consoles 200.

Console 200 can comprise energy delivery module 250 shown, which can comprise one or more modules (e.g., electronic modules) configured to: provide drive signals to energy delivery elements 155; receive signals (e.g., from recorded reflections such as ultrasound reflections) from energy delivery elements 155; or both. Energy delivery module 250 can be configured to drive one, two, or more phased arrays of energy delivery elements 155 comprising ultrasound transducers, such as when providing a drive signal as described in reference to studies S1, S2, S3, S4, and/or S5 described herein.

Energy delivery module 250 can comprise one or more therapy-based drive signal circuitry components, therapy signal generator 255 shown, which can comprise one or more transmit channels (e.g., driver channels configured to drive energy delivery elements 155 to transmit energy such as ultrasound energy and/or other energy). Therapy signal generator 255 can comprise multiple transmit channels. Therapy signal generator 255 can be configured to independently control the phase, amplitude, and/or both phase and amplitude of each transmit channel. Each channel of therapy signal generator 255 can be controlled by processing unit 50 (e.g., computer controlled, such as when processing unit 50 comprises a microcontroller and/or an FPGA). Processing unit 50 can be configured to change (e.g., to dynamically change, such as while treatment energy is being delivered by energy delivery elements 155) the focusing parameters of the energy (e.g., ultrasound beam) being delivered, such as to dynamically focus (or refocus) the treatment energy delivery. Focusing parameters can include the focusing depth, the aperture size, the power output, and/or the focusing angle. The focusing point of the treatment energy delivery can be dynamically adjusted within the field of view of energy delivery elements 155.

Energy delivery module 250 can comprise one or more imaging (e.g., ultrasound imaging) drive circuitry components and/or signal recording components, imaging signal generator 256 shown. Imaging signal generator 256 can comprise multiple transmit channels (e.g., driver channels configured to drive energy delivery elements 155 to transmit energy such as ultrasound energy and/or other energy), and/or imaging signal generator 256 can comprise multiple receive channels (e.g., channels configured to record reflected energy signals (e.g., ultrasound signals) that are sensed by energy delivery elements 155). In some embodiments, therapy signal generator 255 and imaging signal generator 256 comprise one or more of the same components, for example when a transmit channel of therapy signal generator 255 is configured to transmit energy (e.g., ultrasound energy) for both treatment energy delivery and to image tissue (e.g., to deliver treatment energy during a treatment portion, and to deliver imaging energy during an imaging portion of a treatment procedure, as described herein). In some embodiments, therapy signal generator 255 comprises imaging signal generator 256, for example when energy delivery module 250 does not comprise separate transmit channels for delivering treatment and imaging energy, and/or when therapy signal generator 255 also comprises one or more receive channels.

In some embodiments, system 10 includes one or more servers, server 80 shown, where each server 80 can be configured to provide data storage and/or data processing, such as data processing for the providers of system 10 (e.g., the manufacturer and/or distributor of system 10) and/or the users of system 10. As used herein, data processing can refer to: the receiving of data; the filtering, sorting, analysis, and/or other processing of data; the transmission of data (e.g., transmitting the results of data processing); and/or the storage of data, such as data received from multiple consoles 200, multiple treatment devices 100, multiple imaging devices 800, and/or multiple other components of system 10 located at various clinical sites. Server 80 can comprise one or more processing units 50. Additionally, or alternatively, server 80 can include one or more data storage units for storing data collected by system 10, data 85 shown. In some embodiments, server 80 is configured to process data from various users of system 10, for example when the provider of system 10 maintains one or more servers 80 configured to process data for each (and/or a subset) of the users of system 10 (e.g., each of the patients and/or clinicians of system 10). Server 80 can comprise an “off-site” server (e.g., remotely located from the users of system 10), such as a server owned, maintained, and/or otherwise provided by the provider of system 10. Alternatively, or additionally, server 80 can comprise a cloud-based server.

In some embodiments, data 85 includes data recorded during a medical procedure (e.g., a medical procedure performed using system 10), for example data related to one or more energy delivery parameters, and/or one or more patient parameter (e.g., location of tissue receiving energy delivery and/or one or more patient physiologic parameters present when energy was delivered).

In some embodiments, server 80 is configured to communicate with one or more treatment devices 100, such as communication provided via network 75 between communication module 107 and server 80, and/or between communication module 107 and communication module 207 of console 200, where console 200 is configured to communicate with one or more treatment devices 100, and server 80 is configured to communicate with one or more consoles 200 (e.g., to communicate via network 75). In some embodiments, server 80 is configured to collect data 85, for example data 85 comprising usage information (e.g., treatment device 100, and/or imaging device 800 usage information).

As described herein, one or more components of system 10 can comprise all or a portion of processing unit 50, such as a processing unit 50 comprising a processor 51 and memory 52 coupled to the processor 51, where memory 52 stores instructions 53 for processor 51 to perform algorithm 55. In some embodiments, algorithm 55 comprises an AI algorithm, such as when the AI algorithm is trained based on usage data collected by server 80.

System 10 can include one or more assemblies that are configured to alert a user of system 10, alert assembly 40 shown. One or more devices or other components of system 10 can comprise all or a portion of an alert assembly 40, such as when all or a portion of an alert assembly 40 is integral to: treatment device 100, console 200, imaging device 800, and/or another component of system 10. Alert assembly 40 can include one or more alert elements, alert element 49 shown, that provide a visible, audible, tactile, and/or other signal to a user. In some embodiments, alert assembly 40 is configured to provide an alert, via alert element 49, indicating a warning or other alert condition to a user (e.g. an undesired or other event or condition has occurred and/or is present). All or a portion of one or more alert assemblies 40 can be integrated into one, two, or more of the various components of system 10, such as when integrated into treatment device 100, imaging device 800, and/or other component of system 10.

In some embodiments, system 10 is configured to allow a user (e.g., a clinician) to set one or more alert thresholds for a set of one or more parameters that are monitored by the system (e.g., one or more parameters that are monitored by treatment device 100, console 200, imaging device 800, and/or other system 10 component). In these embodiments, when a threshold of a monitored parameter is exceeded, alert assembly 40 can be configured to alert one or more users of system 10, for example the clinician using treatment device 100. In some embodiments, a threshold represents a maximum energy to be applied to tissue, a maximum time for energy delivery, and/or a maximum tissue temperature.

As described herein, one or more components of system 10 can include at least a portion of a processing unit 50, for example processing units 105 and/or 205 of treatment device 100 and console 200, respectively, each comprising at least a portion of a processing unit 50. Various processing units of system 10 can be referred to singly or collectively herein, as processing unit 50. Processing unit 50, for example by performing algorithm 55 via processor 51, can be configured to detect target tissue (e.g., a target location) and/or non-target tissue (e.g., a non-target location) to be imaged, treated (e.g., modulated), or both.

In some embodiments, system 10 is configured to differentiate between a first tissue type and a second tissue type. For example, image data ID collected using energy delivery elements 155 and/or imaging device 800 (e.g., an MRI or other imaging device) can be used by processing unit 50 to differentiate the tissue type. In some embodiments, processing unit 50 is configured to differentiate between healthy tissue and diseased tissue, between modulated tissue and non-modulated tissue, and/or to make another differentiation in tissue.

In some embodiments, one or more components of system 10 are configured to be calibrated. For example, one or more components of treatment device 100, and/or imaging device 800 can be configured to be calibrated.

In some embodiments, system 10 includes one or more pharmaceutical and/or other agents, agent 30 shown, such as one or more agents that may be administered prior to, during, and/or after use of treatment device 100, and/or imaging device 800.

In some embodiments, system 10 includes one or more accessory devices, accessory device 700 shown. Accessory device 700 can comprise an agent delivery system, such as an intravenous or other agent delivery system configured to deliver agent 30 to a patient (e.g., prior to, during, and/or after an energy delivery procedure performed using treatment device 100).

As described herein, system 10 can comprise one or more functional elements, such as functional elements 99, 199, 299, 899, and/or 999 shown. Each functional element can comprise one or more sensors, one or more transducers, and/or one or more other functional elements. Functional element 99, 199, 299, 899, and/or 999 can comprise a heating element and/or a cooling element, such as a thermal element configured to heat or cool the patient (e.g., heat or cool the patient's head or other tissue into which energy is to be delivered). Functional element 99, 199, 299, 899, and/or 999 can comprise a manipulating element, such as a manipulating element configured to manipulate (e.g., robotically manipulate) a component of system 10. Functional element 99, 199, 299, 899, and/or 999 can comprise a vacuum applying element, such as a vacuum applying element configured to maintain a system component (e.g., treatment assembly 150 of treatment device 100) releasably secured to the patient (e.g., to the patient's head) or a surface (e.g., a table).

In some embodiments, system 10 (e.g. via algorithm 55) is configured to perform a “system diagnostic procedure”. For example, system 10 can be configured to assess the functionality of one or more components of treatment device 100. When one or more malfunctioning components of treatment device 100 are identified (e.g., confirmed via a system diagnostic procedure), system 10 can: enter an alert state; disable function of treatment device 100 or another system 10 component; and/or perform another alert function and/or compensation function. System 10 can be configured to perform various system diagnostic procedures.

In some embodiments, system 10 is configured to determine an “angle of orientation” of treatment assembly 150 of treatment device 100 (e.g., an angle of orientation of all or a portion of energy delivery elements 155). System 10 can be configured to produce a two-dimensional (2D) and/or three-dimensional (3D) image, and system 10 can enhance (e.g., correct, adjust, and/or otherwise enhance) a 2D or 3D image based on the determined angle of orientation.

System 10 can be configured to: automatically identify (e.g., via algorithm 55) one or more tissue landmarks; and/or allow an operator to manually identify one or more tissue landmarks. The landmark identification can be used to: automatically position and/or reposition treatment assembly 150; and/or allow an operator to manually position and/or reposition treatment assembly 150. System 10 (e.g., via algorithm 55, such as an AI algorithm) can be configured to continuously and/or intermittently image tissue during a treatment performed using treatment device 100, such as to track the progress of the treatment. System 10 can be configured to continuously or at least repeatedly confirm proper positioning of treatment assembly 150 (e.g., of energy delivery element 155) and/or another system 10 component during modulation and/or other tissue treatment, such as by monitoring changes in target tissue being treated and/or changes in other physiologic parameters of the patient. In some embodiments, functional element 99, 199, 299, 899, and/or 999 comprises a manipulating component configured to automatically manipulate (e.g., robotically manipulate) treatment assembly 150 if changes in target tissue exceed a threshold.

As described herein, treatment device 100, and/or other energy-delivering component of system 10 can be configured to deliver energy (e.g., ultrasound) to treat a patient (e.g., to modulate and/or otherwise treat target tissue of a patient), and system 10 can be configured (e.g. via algorithm 55) to monitor the progress of the treatment. In some embodiments, algorithm 55 is configured to automatically adjust one or more energy delivery settings (e.g., amplitude, frequency, pulse width, and/or duty cycle) if progress of the treatment exceeds a threshold (e.g., to avoid under-stimulation and/or over-stimulation).

System 10 can be configured to: collect a first set of image data ID, and perform a first tissue treatment procedure to treat a first volume of target tissue. System 10 can be further configured to subsequently collect a second set of image data ID, and then perform a second tissue treatment procedure on a second volume of target tissue (e.g., where the second volume of target tissue includes at least a portion of the first volume of target tissue, or when all of the second volume of target tissue is different tissue than the first volume of target tissue). The first and/or second sets of image data ID can be produced using energy delivery elements 155, imaging device 800, and/or another imaging device. The second set of image data can include identification of un-treated tissue that was intended to be treated in the first tissue treatment procedure.

As described herein, system 10 can comprise one or more devices for delivering energy to treat tissue. For example, treatment assembly 150 of treatment device 100, can be configured to deliver energy to tissue to treat the tissue. In some embodiments, treatment assembly 150, and/or another component of system is configured to deliver ultrasound energy to treat the tissue. Alternatively, or additionally, energy delivery by one or more of these components can comprise energy of one, two, or more forms selected from the group consisting of: sound energy such as ultrasound energy; light energy such as laser light energy; thermal energy such as heat energy and/or cryogenic energy; electromagnetic energy such as radiofrequency energy, microwave energy, and/or electroporation energy; chemical energy; mechanical energy; and combinations of one, two, or more of these.

Treatment assembly 150 can be configured to treat tissue (e.g., target tissue) via the delivery of ultrasound energy, as described herein. In some embodiments, system 10 (e.g., energy delivery elements 155 via one or more algorithms 55) is configured to adjust the frequency of ultrasound energy (e.g., ultrasound energy configured to modulate brain or other tissue) delivered by energy delivery elements 155 based on one or more of: distance to the target tissue; tissue type of the target tissue; and/or characteristics of tissue between the ultrasound transducers and the target tissue. System 10 can be configured to deliver energy (e.g., ultrasound energy, magnetic field energy, or both) to multiple targets simultaneously, sequentially, or both. In some embodiments, energy delivered to a first target is delivered with a first set of energy delivery parameters, and energy delivered to a second target is delivered with a second set of energy delivery parameters. The first and second sets of energy delivery parameters (e.g., amplitude, frequency, pulse width, and/or duty cycle) can be a similar set of parameters (e.g., the same values) or a different set of parameters (e.g., one or more differences in amplitude, frequency, pulse width, and/or duty cycle).

System 10 (e.g., via algorithm 55) can be configured to dynamically adjust imaging time, treatment time, or both. Alternatively, or additionally, system 10 can be configured to dynamically adjust the focus of energy delivery (e.g., ultrasound energy delivery) for imaging, treatment, or both.

Target tissue to be treated by treatment device 100 can comprise multiple volumes of tissue, such as multiple adjacent volumes of tissue, and/or multiple non-adjacent volumes of tissue. In some embodiments, multiple volumes of tissue are treated (e.g., via one or more sets of energy delivery transducers of energy delivery elements 155) via a procedure in which one volume of target tissue is treated through an energy delivery (e.g., an ultrasound energy delivery), the focus of energy is then adjusted (e.g., to change the field of view to include the second volume of tissue), and a second volume of target tissue is then treated. In these embodiments, the two volumes of tissue can be treated without translating, rotating, and/or otherwise moving the treatment assembly 150 (e.g., field of view changed via electronic means only). Such avoidance of moving the treatment assembly 150 can increase safety, reduce procedure time, and/or provide other benefits. In some embodiments, changing the electronic focus of one, two, or more treatment assemblies 150, combined with translating, rotating, and/or otherwise moving the treatment assembly 150, is used to treat one, two, or more volumes of target tissue.

An electronic and/or movement-based adjustment of the field of view of one, two, or more treatment assemblies 150 can be performed to compensate for patient movement, such as to keep the field of view of treatment assembly 150 directed toward a particular volume of tissue, as that tissue volumes moves (e.g., during respiration or other cause of tissue movement).

Treatment assembly 150 of treatment device 100 can include one or more sets of energy delivery transducers of energy delivery elements 155 that are configured to deliver ultrasound energy to: produce image data ID; modulate, and/or otherwise treat tissue; or both. In some embodiments, a first set of energy delivery elements 155a is configured to at least produce image data ID, and a second set of energy delivery elements 155b is configured to at least treat tissue.

As described herein, console 200, and/or another system 10 component, can collectively comprise one or more algorithms, algorithm 55 shown, that are stored as instructions 53 in memory 52, for implementation by processor 51. Algorithm 55 can comprise an artificial intelligence (AI) algorithm, also as described herein. In some embodiments, algorithm 55 is configured to produce an anatomical model based on, at least, image data ID. The anatomical model can comprise two or more sets of image data that are “stitched” together by algorithm 55. Algorithm 55 can be configured to produce the anatomical model based on one or more landmarks that are identified in the image data ID. Algorithm 55 can be configured to identify one or more features of interest in the anatomical model. The one or more features of interest can comprise one or more features selected from the group consisting of: tumor tissue; margin tissue; blood vessel; duct; target tissue; safety margin tissue; non-target tissue; and combinations of these.

Algorithm 55 can be configured to identify non-target tissue in an anatomical model produced by system 10, and system 10 can be configured to limit energy delivery (e.g., via treatment device 100) to the non-target tissue using the anatomical model.

Algorithm 55 can be configured to compensate for tissue movement when creating an anatomical model. Image data ID can comprise image data that is collected prior to the occurrence of the tissue movement. Algorithm 55 can be configured to adjust the “trajectory” (e.g., a 2D or 3D movement path) of a system 10 component based on an assessment of the tissue movement. Algorithm 55 can be configured to predict patient movement and/or other tissue movement. System 10 can comprise a second imaging device (e.g., imaging device 800), and algorithm 55 can be configured to create the image data ID by combining first image data received from energy delivery elements 155 and second image data received from the imaging device 800 (e.g., an MRI).

Algorithm 55 can comprise an AI or other algorithm that is configured to identify one or more volumes of target tissue to be modulated and/or otherwise treated by system 10. System 10 can be configured to gather image data ID from an imaging device 800 (e.g., an MRI), and algorithm 55 can be configured to produce an anatomical mode based on the image data ID from the imaging device 800.

Algorithm 55 can comprise a bias. For example, algorithm 55 can be configured to determine one or more energy delivery parameters (e.g., amplitude, frequency, pulse width, and/or duty cycle), and the bias can cause the energy delivery to tend toward under-stimulation, or over-stimulation. Alternatively or additionally, algorithm 55 can be configured to “steer” delivery of energy (e.g., mechanically, electronically, or both), and the bias of algorithm 55 can be configured to cause delivered energy to avoid certain non-target tissues into which energy delivery would potentially result in an adverse event.

Housing 110, treatment assembly 150, and the other components of treatment device 100 can be configured to be “MRI-compatible”, in other words being constructed of materials that enables device 100 to be placed in the imaging view of an MRI during a magnetic resonance imaging procedure without adverse effects (e.g., without undesired heating of a component, without distortion of the MRI image, and the like). In some embodiments, one or more portions of console 200 and/or other system 10 component is MRI-compatible.

System 10 can be configured to provide high-precision energy delivery (e.g., ultrasound energy delivery) that can be used, for example, in mental health or neurological clinics. In some embodiments, system 10 provides mechanisms for controlling the energy dose delivered through to a target region (e.g., delivered through the skull or other bone) to produce predictable effects in the target regions. In some embodiments, system 10 provides multi-focal energy delivery that predictably modulates specific target regions (e.g., brain regions) based on the specific needs of a patient.

System 10 can be configured to deliver focused ultrasound energy, such as transcranial focused ultrasound energy, that provides noninvasive and reversible treatment of a medical disorder via precise and personalized manipulation of one or more brain circuits or other target locations. System 10 can be configured to deliver focused ultrasound (e.g., low-intensity focused ultrasound), such as to non-invasively stimulate a target location of the brain (e.g., a target location of the deep brain). Alternatively or additionally, system 10 can be configured to deliver focused ultrasound (e.g., low-intensity focused ultrasound) to cause the efficacious delivery of one or more agents (e.g., one or more agents 30 comprising pharmaceutical agents), such as to deliver the one or more agents across an intact, and transiently opened, blood-brain barrier. System 10 can deliver focused ultrasound through an intact skull and scalp and into specified deep brain regions (e.g., regions spanning less than or equal to 10 mm, 5 mm, 4 mm, or 3 mm in diameter when high precision is desired, or regions greater than 3 mm, 4 mm, and/or 5 mm when high precision is not desired). Treatment assembly 150 can include energy delivery elements 155 comprising one, two, or more arrays of ultrasound transducers, and these arrays can stimulate multiple different target locations (e.g., brain locations) sequentially (e.g., in fine temporal sequences), simultaneously, or both. These arrays can deliver focused ultrasound with high spatiotemporal resolution (e.g., with spatial accuracy of no more than 1.0 mm, 0.7 mm, and/or 0.5 mm, temporal accuracy of no more than 0.05 μs, 0.03 μs, and/or 0.01 μs, or both). The location into which the ultrasound energy is focused can be changed manually (e.g., via movement of the patient and/or via movement of one or more portions of energy delivery elements 155), and/or it can be changed programmatically by system 10 (e.g., via electronic focusing performed by processing unit 50).

Treatment assembly 150 can comprise energy delivery elements 155 comprising one or more arrays of one or more energy delivery transducers (e.g., ultrasound transducers) that can provide an energy delivery pathway that can be electronically steered (also referred to as “focused” herein). System 10 can comprise an imaging device 800 (e.g., an MRI) configured to be used in enhancing the delivery of energy by treatment assembly 150. In some embodiments, treatment device 100 and the other components of system 10 are configured to minimize the use of imaging device 800 (e.g., minimize the use of an MRI).

System 10 can be configured to deliver energy (e.g., ultrasound energy) to a target location (e.g., one or more brain locations), and to compensate for the attenuation, dephasing, and/or other changes to the delivered energy that can occur in the pathway of the energy delivery to the target location. For example, the energy delivery pathway can include the skull or other bone, the scalp, hair, and/or other “obstacles” that may impact the energy being delivered. Obstacles in which compensation can be beneficial can also include the coupling between treatment module 150 and the patient's head, as well as any bubbles that may be present in the coupling interface. A compensation performed by system 10 can comprise determining amplitude, timing (e.g., frequency, pulse width, phase, and/or other timing parameters), and/or other energy delivery parameters (e.g., ultrasound energy delivery parameters) such that the delivered energy reaches a selected target location with an intended intensity (e.g., an intensity that provides a therapeutic benefit without adverse effects). The compensation performed by system 10 can include a test measurement performed using the skull and other obstacles of the patient to receive the therapy (e.g., not an estimate), using a similar form (e.g., similar frequency) to the intended therapeutic energy to be delivered subsequently. As described herein, treatment assembly 150 can comprise multiple [171] portions, such as a first portion, assembly 150a and a second portion, 150b. Treatment assembly 150a can comprise a first energy delivery elements 155a comprising one or more energy delivery transducers (e.g., one or more ultrasound transducers), and treatment assembly 150b can comprise a second energy delivery elements 155b comprising one or more energy delivery transducers (e.g., one or more ultrasound transducers). Each of energy delivery modules 155a and 155b can comprise one or more transducers that are configured to deliver energy (e.g., deliver ultrasound energy), receive energy (e.g., receive ultrasound energy), or both. Treatment assembly 150a can be positioned on one side of a target location to be treated, and second portion 150b can be positioned on an opposite side of the target location. In some embodiments, compensation provided by system 10 can be based on “relative through-transmit” (RTT) measurements made between assemblies 150a and 150b, that directly measures the attenuation and distortion of a given energy delivery pathway (e.g., a pathway through the skull and scalp of the same patient to receive the therapy). System 10 then takes these measured attenuation and phase values and compensates for them by adjusting the amplitude and phase for the transducers of energy delivery elements 155 that are to deliver the therapeutic energy delivery. The measurements of the energy delivery (e.g., ultrasound energy delivery) skull aberrations using the intended energy delivery itself provide accurate compensation for the delivered energy intensity into specified target locations (e.g., brain targets), sharpen the energy delivery focus, and so lead to more precise, safer, and more effective treatments for the patient. This compensation can be implemented via electronic hardware of controller 50, and the compensation can accurately achieve an intended intensity of energy delivery (e.g., as determined by a clinician of the patient) to a target location (e.g., one or more locations of the patient's brain).

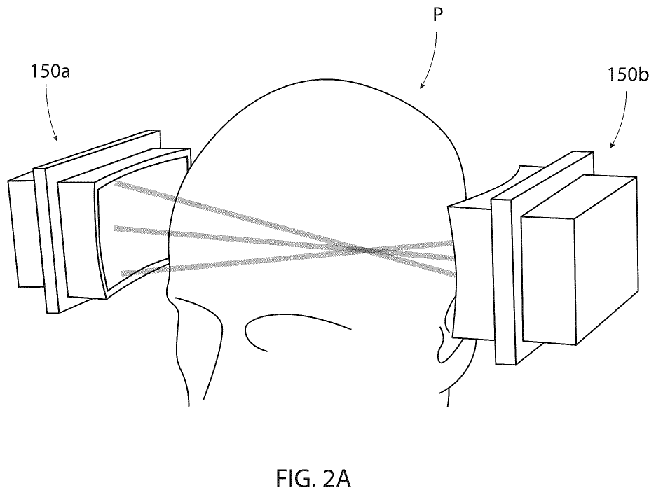

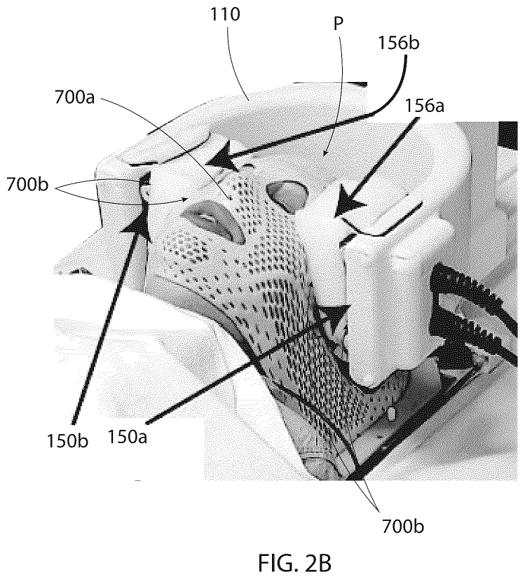

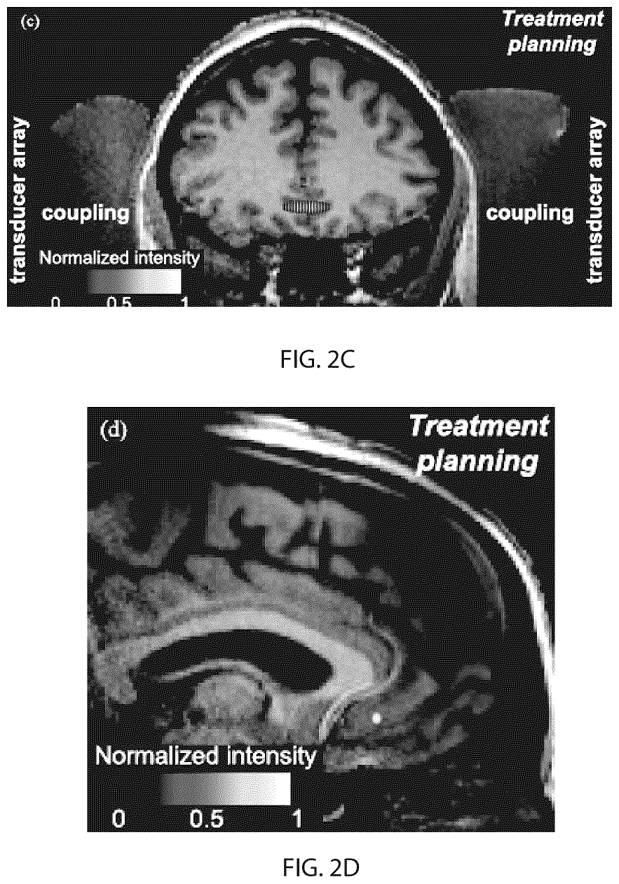

As described herein, treatment device 100 can comprise a first treatment assembly 150a configured to be positioned on one side of a patient's head, and a second treatment assembly 150b configured to be positioned on the opposite side of the patient's head, for example as described in reference to FIGS. 2A, 2B, and/or 13B, and/or in reference to studies S1, S2, S3, S4, and/or S5. In these embodiments, treatment device 100 can be configured as an energy delivery device configured to deliver ultrasound energy to the patient's brain, such as when energy delivery elements 155 comprises 64 to 1,024 ultrasound transducers (e.g., approximately 256 ultrasound transducers) that are positioned within a housing 110. The locations of the transducers used to deliver the ultrasound energy can be optimized for a particular multi-focal operation in a specific patient. The delivery of ultrasound into deep brain targets is possible owing to minimal attenuation of ultrasound by brain tissue. The head and the skull in particular, however, de-phase and attenuate ultrasound waves. Accordingly, system 10 can use ultrasound itself to correct (also referred to as “compensate”) for the aberration of ultrasound by the skull and other obstacles of the head as described herein. In this configuration, the head aberrations are measured directly and accurately and do not require additional head scans such as CT or MRI. As described herein, system 10 can perform ultrasound RTT measurements through each respective segment of the given head, these measurements providing the phase and amplitude values that can be used to correct for the aberrations of each intended energy delivery path of the particular patient's head. The amplitudes of drive signals provided by energy delivery module 250 to the respective ultrasonic transducers of energy delivery elements 155 are scaled and the phases shifted such as to deliver an undistorted, deterministic intensity into a treatment target as part of a therapy for the patient. In some embodiments, specific features of the RTT waveform can be optimized to maximize the accuracy of the detection of the ultrasound energy delivered through the head and, thus, the accuracy of the compensation of the therapeutic delivery into the head.

Compensation performed by system 10 can comprise at least one ultrasound transducer of a first treatment assembly 150a being driven (e.g., via energy delivery module 250) to generate ultrasound waves that achieve an intended ultrasound energy at a target location in a “free field volume”, which corresponds to the target location of energy delivery (e.g., a target brain location). Ultrasound waves exiting the free field volume are measured by at least one receiving ultrasound transducer of a second treatment assembly 150b that is positioned on a opposite side of the free field volume at a fixed distance and orientation relative to first treatment assembly 150a (e.g., relative to the at least one transmitting ultrasound transducer of first treatment assembly 150a). Treatment assembly 150a and 150b can be part of a treatment device 100 comprising a device that is mounted to a surface, such as a table, bed, desk, or other surface-containing component. A portion of the patient's body, such as the patient's head in brain stimulation procedures, can be positioned between the first treatment assembly 150a and the second treatment assembly 150b and the transmitting ultrasound transducers of energy delivery elements 155a are again driven to generate the same ultrasound waves into the head and the receiving ultrasound transducers of energy delivery 155b measure the ultrasound waves exiting the head, the exiting ultrasound waves having been altered due at least in part to a presence of a head in an ultrasound path between the transmitting ultrasound transducers of energy delivery elements 155a and the receiving ultrasound transducers of energy delivery elements 155b. One or more adjusted ultrasound waveforms are then determined (e.g., by algorithm 55) based on differences between the measured ultrasound waves through the free field volume and the measured altered ultrasound waves through the head, wherein the adjusted ultrasound drive signals to be provided by energy delivery module 250 compensate for the attenuation and phase shift in order to deliver an intended ultrasonic stimulation energy at the target location. The transmitting ultrasound transducers of energy delivery elements 155a of treatment assembly 150a are then driven by the adjusted drive signals provided by energy delivery module 250 to generate the adjusted ultrasound waves into the head.

In some embodiments, system 10 can be configured for treatment of anxiety and depression-related disorders, including post-traumatic stress disorder. In these embodiments, the target location for energy delivery can comprise one, two, or more anatomical locations selected from the group consisting of: nucleus accumbens; cingulate cortex; other brain location; and combinations of these. These disorders involve aberrant connectivity of two deep brain regions: subgenual cingulate and the amygdala, with neighboring circuits. For these and other medical conditions (e.g., addiction and/or other medical conditions), system 10 can be configured to provide low-intensity ultrasound (e.g., peak intensity less than or equal to 300 W/cm², 225 W/cm², and/or 190 W/cm²), for a desired time period (e.g., a time period of no more than 3 hours, 2 hours, and/or 1 hour), the energy (e.g., ultrasound energy) being delivered into a target location comprising the cingulate and the amygdala. The energy delivery is configured to induce durable changes in the associated circuits.

In some embodiments, system 10 can be configured for treatment of pain. For example, system 10 can be configured for treatment of thalamic nuclei, such as those involved in pain. Pain disorders can involve aberrant connectivity of thalamic nuclei insular cortex, cingulate cortex, the nucleus accumbens, and/or the ventral tegmental area. For these medical conditions, system 10 can be configured to provide low-intensity ultrasound (e.g., peak intensity less than or equal to 300 W/cm², 225 W/cm², and/or 190 W/cm²), for a desired time period (e.g., a time period of no more than 3 hours, 2 hours, and/or 1 hour), the energy (e.g., ultrasound energy) being delivered into a target location comprising these brain circuits, such as to modulate pain thresholds.

In some embodiments, the targeting of a target region (e.g., one or more target regions of the brain) can be confirmed (e.g., in a closed loop arrangement) using fMRI BOLD (Blood-Oxygen-Level-Dependent imaging), MRI thermometry or MRI acoustic radiation force imaging (e.g., when imaging device 800 comprises an MRI). These imaging sequences visualize the region impacted by the energy delivery (e.g., ultrasound energy delivery) and thus can increase the reproducibility of the therapies provided by system 10, as well as minimize potential off-target effects. In some embodiments, an imaging device 800 comprising an MRI can be used to establish patient-specific anatomy (e.g., patient-specific anatomy of the head and the brain).

System 10 can be configured to allow an operator (e.g., a clinician) to select one or more target locations (e.g., deep brain location), into which energy (e.g., ultrasound energy) is to be delivered, such as to deliver therapeutic energy to the target location, image the target location, or both. In some embodiments, system 10 is configured to automatically, or semi-automatically (“automatically” herein) produce a treatment plan for patient treatment, such as a treatment plan that includes one or more target locations for treatment.

In some embodiments, a treatment is performed by system 10 via treatment device 100 comprising a delivery of energy (e.g., ultrasound energy) by one or more treatment assemblies 150 in which tissue is ablated. In some embodiments, a tissue ablation procedure is performed based on data collected during a compensation procedure (e.g., as described herein) that was performed prior to and/or during the ablation procedure.

In some embodiments, system 10 is configured to provide diagnostic information that can be used to guide the implantation of an accessory device 700 comprising an implant, such as an implant comprising a deep brain stimulator.

In some embodiments, system 10 is constructed and arranged as described in United States Patent Application US20230210493A1, Ser. No. 18/093,220, titled “System and Methods for Modulation of Deep Brain Circuits”, filed Jan. 4, 2023, and/or International PCT application WO2023211898, Serial Number PCT/US2023/019759, titled “System and Method for Sharpening the Focal Volume of Therapeutic and Imaging Systems”, filed Apr. 25, 2023, the contents of each of which is incorporated herein by reference for all purposes.

Referring now to FIG. 1B, another embodiment of a system for performing a medical procedure is illustrated, consistent with the present inventive concepts. System 10 of FIG. 1B can be of similar construction and arrangement as system 10 of FIG. 1, FIG. 1A, and/or otherwise described herein. In the embodiment of FIG. 1B, system 10 includes treatment device 100, as well as one or more other components of system 10 of FIG. 1A, as well as a second treatment device 900 as shown. Treatment device 100 can be configured to deliver ultrasound energy (e.g., deliver stimulating ultrasound energy to tissue of a patient, as described herein), and second treatment device 900 can be configured for delivering another (e.g., different) form of energy, such as magnetic field energy (e.g., device 900 comprises a device configured to produce a strong magnetic field within tissue of a patient). Second treatment device 900 comprises treatment assembly 950 for delivering the second form of energy (e.g., magnetic field energy). Second treatment device 900 can comprise functional element 999, a functional element comprising one or more sensors, one or more transducers, and/or one or more other functional elements as described herein. In some embodiments, second treatment device 900 comprises an MRI and/or other magnetic field generating device. In some embodiments, second treatment device 900 comprises one or more of: a magnetic field generating component; a permanent magnet; a magnetic coil; and/or combinations of one, two, or more of these. In some embodiments, treatment device 100, second treatment device 900, and/or another component of system 10 is constructed and arranged as described in reference to study S3 described herein.

In some embodiments, treatment device 100 comprises second treatment device 900, for example a single device can be configured to delivery two forms of energy in performing a medical procedure (e.g., a treatment in which brain tissue or other tissue is stimulated) on a patient, such as a single device configured to deliver ultrasound energy and magnetic field energy.

System 10 of FIG. 1B can be configured to generate electric fields (e.g., focal electric fields) by combining two energy deliveries, magnetic field delivery by device 900 and ultrasound field delivery by device 100. For example, system 10 can be configured to combine two orthogonal, remotely applied energies comprising magnetic fields and focused ultrasound fields. The effect of this energy delivery derives from the Lorentz force equation as applied to magnetic and ultrasonic fields. Studies were performed in which this effect was elicited using system 10 (reference study S3 described herein), and it was confirmed that the generated electric fields align with the Lorentz equation. In various studies, the effect significantly and safely modulated peripheral nerves in human subjects, as well as deep brain regions of non-human primates. This configuration of system 10 opens a new set of applications in which electric fields are generated at high spatiotemporal resolution within intact biological tissues or materials, thus circumventing the limitations of traditional electrode-based procedures.

Electromagnetic waves delivered by previous devices cannot be used to modulate deep brain targets in a focal manner. At high frequencies (light or infrared), electromagnetic waves are severely attenuated by the skull or superficial tissue layers. At lower frequencies, the waves (microwaves) can penetrate into depth, but microwaves at the relevant neuromodulatory doses damage mitochondria and possibly other cellular structures. At yet lower frequencies (radio range), the wavelength is too broad—dozens of centimeters or meters—to allow for focal stimulation.

Ultrasonic waves delivered by system 10 provide both depth penetration and safe application. The ultrasound delivered by system 10 can effectively modulate excitable cells at high frequencies (e.g., frequencies of at least 10 MHz), at which there are strong radiation forces that mechanically displace membranes and activate ion channels. However, ultrasound at such high frequencies is severely attenuated by the human skull; for this reason, frequencies below 1 MHz can be used for transcranial therapies. Ultrasound delivered by system 10 can modulate excitable structures also at lower frequencies, but strong effects that are based on established biophysical principles, remain elusive.

System 10 of FIG. 1B can be configured to produce strong magnetic fields that are used to noninvasively generate localized electric fields, such as by combining focused ultrasound and a magnetic field. Specifically, when a charged molecule q moves at a velocity \vec{v} in magnetic field \vec{B}, the molecule experiences the Lorentz force \vec{F}=q(\vec{v}×\vec{B}) with intensity

$$\vec{E} = \frac{\vec{F}}{q} = \vec{v} \times \vec{B} .$$

To produce localized electric field, the molecular motion \vec{v} should occur only in the target of interest.

System 10 can achieve this targeting using focused ultrasonic waves. Ultrasound, a mechanical pressure wave, displaces molecules at its target with velocity,

$$v = \frac{P}{Z}$$

where Z is a constant of the medium, “acoustic impedance”. Thus, acoustic waves delivered by system 10 into a target perpendicular to a magnetic field produce in the target electric field intensity

$$E = \frac{PB}{Z} .$$

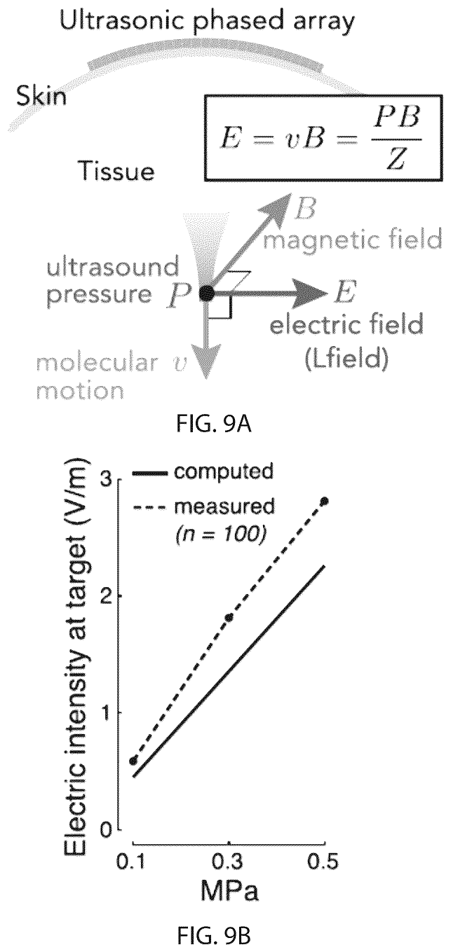

This intensity points in the direction that is perpendicular to both constituents (reference FIG. 9A). As a consequence of this electric field, positively and negatively charged molecules are pulled in opposite directions, inducing electric currents. Sound waves alone would displace positively and negatively charged molecules in the same direction, thus no gradient of charge and so no electric field would be created; the magnetic field is a critical addition. The temporal profile of the evoked field E(t) corresponds to

$$E ( t ) = \frac{P ( C ) B ( C )}{Z} .$$

Therefore, the Z induced waveform can be controlled by the temporal profile of the ultrasonic field, the magnetic field, or both (reference FIG. 9B).

System 10 of the present inventive concepts (e.g., system 10 described in reference to FIGS. 1, 1A, 1B and/or otherwise herein) can be configured to treat one, two, or more medical conditions of a patient, including but not limited to: pain; depression, anxiety, addiction, and/or other psychological disorder; Alzheimer's disease; and/or other medical condition. Treatment device 100 of system 10 can comprise a treatment assembly 150 comprising one or more energy delivery elements 155. Energy delivery elements 155 can comprise ultrasound transducers and/or other energy delivery elements in a phased array or other arrangement, such as to treat the medical condition(s) of the patient via delivery of ultrasound energy and/or other energy to one, two, or more target locations (e.g., sequentially or simultaneously), such as one, two, or more target locations of the patient's brain.

In some embodiments, treatment device 100 is configured to deliver ultrasound energy. Treatment device 100 can be further configured to deliver magnetic field energy. Alternatively, or additionally, a component of system 10 (e.g., a second treatment device 100) can be configured to deliver the magnetic field energy. The magnetic field provided can comprise a magnetic field of at least 0.5 T.

In some embodiments, treatment device 100 comprises a first treatment assembly 150a configured to deliver energy and to be positioned on the right side of the head of the patient, and a second treatment assembly 150b configured to deliver energy and to be positioned on the left side of the head of the patient. System 10 (e.g., treatment device 100) can further comprise a housing 110 that is configured to position the first and second treatment assemblies 150a,b to each deliver the energy through the temporal bone window of the patient's skull.

In some embodiments, system 10 is configured to deliver energy to multiple different target locations of the deep brain without moving treatment device 100, without moving the patient's head, or without moving neither the head nor device 100. System 10 can be configured to deliver energy to at least a first target location and a second target location in a sequential arrangement, simultaneously, or both.

In some embodiments, system 10 comprises a housing 110 and a patient mask 700a that are collectively configured to reproducibly position the patient's head in a desired arrangement for delivery of energy, such as to avoid the need for MRI-based guidance (e.g., MRI is used in an initial data gathering procedure only, or not at all).

In some embodiments, system 10 is configured to validate the targeting of energy delivery (e.g., validate that treatment device 100 can deliver energy accurately to one or more target locations) via use of MRI blood-oxygen-dependent (BOLD) imaging. The BOLD images can provide target location guidance information, dosimetry information, or both.

In some embodiments, system 10 further comprises an algorithm 55 configured to determine energy delivery drive signals that compensate for one or more obstacles present in the path of energy delivery from the treatment device to the target location. The one or more obstacles can comprise an obstacle selected from the group consisting of: the skull; the scalp; hair; a component of system 10; and combinations thereof.

A method of using system 10 to treat a medical condition can comprise: selecting a patient for treatment; selecting a system of the present inventive concepts; and delivering energy (e.g., ultrasound energy, or ultrasound energy and magnetic field energy) to one or more target locations of the patient via treatment device 100 to treat the patient's medical condition. The medical condition treated using system 10 can comprise depression, such as when the target location receiving the energy comprises an anatomical location selected from the group consisting of: subcallosal cingulate cortex; nucleus accumbens; cingulate cortex; other brain locations; and combinations of one or more of these. The medical condition treated using system 10 can comprise pain, such as chronic pain, such as when the target location receiving the energy comprises an anatomical location selected from the group consisting of: anterior cingulate cortex; other brain location; and combinations of these. The medical condition treated using system 10 can comprise addiction, anxiety, and/or another psychological disorder, such as when the target location receiving the energy comprises an anatomical location selected from the group consisting of: subcallosal cingulate cortex; nucleus accumbens; cingulate cortex; other brain location; and combinations of these. The medical condition treated using system 10 can comprise cognitive decline, such as mild cognitive decline. The patient selected for treatment of cognitive decline can have a Montreal Cognitive Assessment score of no more than 25. The patient selected for treatment of cognitive decline can receive energy delivery to one, two, or more locations selected from the group consisting of: hippocampus; amygdala; entorhinal cortex; cingulate cortex; fornix; other brain location; and combinations of these. The medical condition treated can comprise Alzheimer's disease. The patient selected for treatment of Alzheimer's disease can have a Montreal Cognitive Assessment score of no more than 15. The patient selected for treatment of Alzheimer's disease can receive energy delivery to one, two, or more locations selected from the group consisting of: hippocampus; amygdala; entorhinal cortex; cingulate cortex; fornix; other brain location; and combinations of these.

Referring now to FIG. 2A-B, an anatomical schematic and a photograph, respectively, of a portion of a system for delivering energy to a brain of a patient is illustrated, consistent with the present inventive concepts. Referring additionally to FIGS. 2C-D, coronal and sagittal views, respectively, are illustrated, of a patient-specific brain anatomy. System 10 can be configured to determine computed fields that are shown superimposed on the patient-specific brain anatomy, such as to label a target location of treatment for a clinician (subgenual cingulate cortex in the views of FIG. 2C-D). As described herein, system 10 can be configured to compensate for the skull and other obstacles present in an energy delivery pathway to a target location of the brain, for example by delivering energy (e.g., ultrasound energy) through a particular pathway with limited obstacles, and/or by compensating for the effects of the obstacles in the pathway, each as described herein. The human skull poses a formidable barrier for ultrasound. For example, the intensity of neuromodulatory ultrasound is attenuated by human skull alone by a factor of 4.5 to 64, depending on skull segment and the individual patient. System 10 can comprise a treatment device 100 that is configured to be positioned about the head of a patient P (as shown in FIGS. 2A-B), such that treatment assembly 150 can deliver energy (e.g., ultrasound energy) through the temporal and parietal skull windows, such as to minimize the severity of the aberrations, both in terms of the ultrasound dephasing and its attenuation caused by passing through the skull. As shown in FIG. 2B, housing 110 can comprise a frame (e.g., an MRI-compatible frame) that positions treatment assembly 150a and 150b in particular anterior-posterior and superior-inferior axes, in order to achieve the desired energy delivery pathway. In some embodiments, system 10 comprises an accessory device, mask 700a, as shown in FIG. 2B, the mask 700a comprising a mask, such as a radiological mask or other mask that includes lateral windows (not shown) enabling unobstructed transmission of ultrasound waves from energy delivery module 250 and into the patient. Mask 700a can be used to immobilize a patient during a procedure performed using system 10. In some embodiments, mask 700a is configured to attach to (e.g., fixedly and/or adjustably attach to) housing 110, such that the relative position between housing 110 and treatment assembly 150 can be maintained (e.g., when mask 700a is applied to a patient, the relative position between the patient and treatment assembly 150 can be maintained, such as to perform a registration procedure as described herein). In some embodiments, treatment device 100 comprises one or more energy-coupling elements, couplers 156a and 156b shown in FIG. 2B, which can be configured to provide an efficient energy delivery pathway (e.g., ultrasound energy delivery pathway) between a treatment assembly 150 and the patient. Couplers 156a and 156b can comprise poly(vinyl alcohol) cryogel, other hydrogel, and/or another substance that provides an efficient energy delivery pathway.

Treatment assembly 150 can comprise energy delivery elements 155 comprising multiple independently controllable energy delivery transducers. In some embodiments, energy delivery elements 155 comprise ultrasound transducers, USTs 155U, such as at least 2, 6, and/or 10 ultrasound transducers, and/or no more than 10,000, 1,000, and/or 252 ultrasound transducers (e.g., two sets of 126 independently controllable ultrasound transducers). USTs 155U can comprise two or more arrays of ultrasound transducers (e.g., at least two phased arrays), that maximize the volume of the target location into which the ultrasound energy can be refocused. This configuration allows an operator (e.g., a clinician) to rapidly treat multiple brain locations precisely, without the need to move the patient nor the treatment device 100. USTs 155U can comprise a first set of ultrasound transducers 155Ua comprising a first spherical phase array of ultrasound transducers, and a second set of ultrasound transducers 155Ub comprising a second spherical array of ultrasound transducers.

Treatment assembly 150a and 150b can be separated by a distance of at least 10 mm, 25 mm, and/or 50 mm, and/or a separation distance of no more than 10,000 mm, 2500 mm, and/or 1000 mm, such as a separation distance of approximately 187 mm. All or a portion of the transducers of USTs 155U can comprise a surface area of at least 0.04 mm², 0.35 mm², and/or 1 mm² (e.g. a transducer of 1 mm by 1 mm), and/or all or a portion of the transducers of USTs 155U can comprise a surface area of no more than 3600 mm², 2025 mm², and/or 900 mm² (e.g., a transducer of 30 mm by 30 mm), such as a transducer with a square surface geometry of 6 mm by 6 mm. USTs 155U can comprise PZT Material. All or a portion of the transducers of USTs 155U can be driven at a frequency of at least 10 kHz, 25 kHz, and/or 50 kHz, and/or of no more than 10,000 kHz, 4,000 kHz, and/or 2,000 kHz, such as a frequency of approximately 650 kHz. All or a portion of USTs 155U can be rated for an acoustic power of at least 0.01 W/cm², 0.05 W/cm², and/or 0.1 W/cm², such as an acoustic power rating of approximately 3 W/cm². USTs 155U can comprise one or more spherically focused arrays (e.g., two spherically focused arrays) with a radius of at least 10 mm, 25 mm, and/or 50 mm, and/or no more than 5,000 mm, 2,500 mm, and/or 1000 mm, such as a radius of approximately 165 mm. In some embodiments, an array of USTs 155U comprise 126 ultrasound transducers in a 9 by 14 grid, with inter-transducer spacing of 0.5 mm. In other embodiments, an array of USTs 155U comprises 130 ultrasound transducers in a 10 by 13 grid, with inter-transducer spacing of 0.5 mm. One or more arrays of USTs 155U can comprise one or more dimensional parameters selected from the group consisting of: a height of 55 mm, a width of 86 mm; a spanning surface area of 47.3 cm² and combinations of two or three of these. The USTs 155U can be connected with a conductor (e.g., an insulated copper wire of approximately 38 gauge), that terminates in an impedance-matching network (e.g., of energy delivery module 250) that is matched at the drive frequency of the USTs 155U (e.g., matched at a 650 kHz drive frequency).

Energy delivery module 250 can comprise 256 output channels, and it can include a high-voltage dual 600 W DC power supply. USTs 155U can be connected to the delivery module 250 by a cable (e.g. a cable of at least 3 m, 6 m, or 9 m), that is detachable from module 250 (e.g., such that it can be passed through MRI waveguides).

Coupling of treatment assemblies 150 to the patient can include the use of couplers 156 shown in FIG. 2B and described herein. In some embodiments, an accessory device, coupling gel 700b comprising an ultrasound coupling gel can be applied between the patient and one or more components of system 10 (e.g. between the patient and a coupler 156) and/or between two or more system 10 components (e.g., between a coupler 156 and a treatment assembly 150).

System 10 can be configured to mechanically register treatment device 100 to a patient, such as to mechanically register treatment device 100 to the patient's brain anatomy. For example, mask 700a can comprise a thermoplastic mask (e.g., Aquaplast U-Frame, QFix or similar) that includes lateral windows for unobstructed ultrasound propagation. Once the patient's head is fixed using mask 700a, a single T1 MRI image can be produced, with the treatment assemblies 150a and 150b mounted on the patient over the brain target of interest. The MRI field of view can include both the patient's brain anatomy and the assemblies 150a and 150b (e.g., assemblies 150 comprising marker 115 comprising fiducial markers visible on the MRI image). Using the fiducial markers, the geometry of the USTs 155U of the treatment assemblies 150a and 150b can be registered within the MRI image of the patient's brain. Housing 110 can comprise horizontal and vertical tracks, and housing 110 can be locked in specific discrete positions. This adjustable, lockable configuration allows the treatment assemblies 150 and associated USTs 155U to be placed repeatedly over any section of the left and right sides of the patient's head for targeting of nearly the entire deep-brain volume. In subsequent treatment sessions (e.g., repeated and similar treatment sessions), a clinical setting outside of the MRI setting can be used, where the treatment assemblies 150 are locked (e.g., manually and/or automatically locked) in the same position as was used during the initial procedure performed in the MRI setting (e.g., where a similar mask 700a is used to position the patient's head in the same location with respect to the treatment assemblies 150a and 150b). This setup procedure reproduces the position of the treatment assemblies 150a and 150b and the subject's head in the same configuration that was performed initially (e.g., and imaged inside the MRI). The anatomical MRI data of this configuration with the registered treatment assemblies 150 can be used in all subsequent “repeat” treatment sessions for that patient, without requiring presence of an MRI, yet achieving precise, patient-specific stimulation of one or more brain target locations for energy delivery.

System 10 (e.g., energy delivery module 250) can be configured to perform beamforming with ultrasound phased arrays of USTs 155U, by emitting ultrasound from each transducer of USTs 155U with delays such that the wavefronts arrive at the intended target location at the same time, such that the wavefronts constructively interfere. System 10 can calculate these delay values via a calculation in which the distance from each transducer to the target location is divided by the speed of sound over the associated acoustic path. The distance from each transducer to the brain target is known from the MRI image produced while treatment assembly 150 was mounted on the patient, where both the USTs 155U and the subject's brain are in the same image space. System 10 can use the speed of sound in water to set the initial delays to focus into a target location, and add estimated phase shifts from any skull compensation performed (e.g., as described herein) to account for phase shifts induced by coupling, hair, scalp, skull, and brain. These delays can be applied, comprising the sum of the focusing in water and the estimated phase shifts, to focus into the target location in the brain.

In some embodiments, treatment device 100 can comprise a treatment assembly 150 with a dual array of USTs 155a. RTT measurements can be performed (e.g., as described herein) in order to assess how a patient's skull and other obstacles distort ultrasound transmissions to a target location of a brain, and to compensate for these distortions. Each individual transducer (e.g., each of 126 transducers) of USTs 155Ua of a treatment assembly 150a can emit a pulse (e.g., a 10-cycle, 650 kHz pulse) while recording responses from all the other, non-transmitting transducers UST 155U on the opposing treatment assembly 150b.

During the through-transmit scans, the peak pressure amplitude of each transmitting transducer can be 80 kPa. The entire process of this scan can take less than one second to complete. The pulse frequency (e.g., 650 kHz pulse frequency) is the same as that to be used in the subsequent, therapeutic neuromodulation procedure.

This through-transmit measurement is relativistic: it is performed with the patient present and absent. When the patient is absent, the measurement can be made through a liquid (e.g., water). This patient-absent measurement requires no prior information about the patient's anatomy, for example it does not require any CT nor MRI images of the patient's head. sᵢⱼ(t) can represent the signal received on a transducer i of treatment assembly 150b, after a brief, 10-cycle pulse is emitted from a transducer j of treatment assembly 150a. The signals received in free field and through the acoustic barriers (skull, hair, coupling, and other barriers) are denoted as

$${s}_{ij}^{F} ( t ) and {S}_{{i}_{j}}^{B} ( t ) ,$$

respectively.

The compensation performed by system 10 can compare the through-transmit measurements in water,

$${s}_{ij}^{F} ( t ) ,$$

with those taken though the subject,

$${S}_{{i}_{j}}^{B} ( t ) ,$$

to estimate the phase shift, τᵢ, and amplitude, Aᵢ, distortions introduced by the subject's head for each transducer i. The through-transmit attenuation from transducer j to transducer i, Aᵢⱼ, can be measured by dividing the peak amplitudes of the through-transmit waveforms through the subject versus through water, when there is no barrier to transmission:

$${A}_{ij} = \frac{max ( {s}_{ij}^{β} ( t ) )}{max ( {s}_{ij}^{F} ( t ) )} \qquad ( 1 )$$

The attenuation through the two opposite sides of the skull are multiplicative, hence Aᵢⱼ=AᵢAⱼ, where Aᵢ is the attenuation factor for transducer i, and the value estimated in order to compensate for the skull and other barriers in front of this transducer. To estimate this value for transducer i, through-transmit pairs can first be selected, where the angle between the transmitting transducer i and the target location, and the transmitting transducer i and the receiving transducers is less than or equal to 7 degrees. Aᵢ is then estimated as the square root of the median through-transmit value between i and the selected receive transducers. Phase correction is estimated similarly by finding the phase shifts τᵢⱼ that minimize the cross correlation between the waveforms

$${s}_{ij}^{F} ( t ) and {s}_{ij}^{B} ( t ) .$$

Once the phase and amplitude values are estimated, the stimulation parameters can be adjusted (e.g., system 10 can automatically adjust the stimulation parameters) to compensate for the distortions. With the attenuation values Aᵢ estimated for each transducer, the voltage applied can be scaled to transducer i by 1/Aᵢ, in order to compensate for this attenuation. If Aᵢ is less than a threshold value of 0.1, the transducer can be turned off (e.g., to avoid overdriving the transducer). Similarly, with the speed through the skull estimated as τᵢ, the emission time of this transducer can be delayed by the same duration, such that the waveforms compensate for this distortion. In some embodiments, the compensation can be performed in less than five minutes, such as less than 3 minutes and/or approximately 2 minutes.

System 10 can be configured to deliver energy (e.g., ultrasound energy) to the subcallosal cingulate cortex and/or associated circuits (noting that severe forms of depression have been linked to hyperactivity of the subcallosal cingulate cortex).

Applicant has conducted various human clinical studies, non-human mammal clinical studies, and other studies, using the systems, devices, and methods of the present inventive concepts.

### Study S1

HUMAN SUBJECTS: In one study, study S1, participants (also referred to as “patients” or “subjects” herein) were selected that had a primary diagnosis of major depressive disorder or bipolar disorder and a self-rated 16-item Quick Inventory of Depressive Symptomatology (QIDS) total score greater than 10. Two patients, patient PS1-1 of 32 years of age, and patient PS1-2 of 35 years of age, both with a history of severe treatment-resistant depression, were recruited in the study.

NEUROMODULATION PARAMETERS: The effects on mood reported in this study were measured in a sonication session (an ultrasound delivery using system 10 as described herein), the sonication session performed outside of MRI. USTs 155U of treatment assembly 150a delivered ultrasound into each target in 30 ms ON periods (650 kHz, 1.0 MPa peak pressure, estimated using the relative through transmit skull correction method described herein, MI=1.2, I_{SPPA}=31 W/cm²) followed by 4 s OFF periods (0.75% duty, I_{SPTA}=0.233 W/cm²). Sonication duration varied from 60 s to 180 s.

ACTIVE SHAM: Potential artifacts that can be associated with the application of ultrasound through the head were controlled. Specifically, an active sham condition was developed that used the same waveform and emission voltages but was not focused into a specific target. The ultrasound emission times were set such that each transducer of UST 155U emitted an unfocused, planar wave from the face of the transducer in the axial dimension. The power applied to the transducer and the pressure emitted from each transducer was the same as during the verum stimulation. This way, the patient's head experienced equal energies and waveforms in both the verum and the active sham conditions, yet the spatial peak pressure inside the brain for the plane wave (0.098 MPa, 0.30 W/cm²) were an order of magnitude lower than the focused condition (1 MPa, 31.1 W/cm²).

EVALUATION OF EFFECTS ON MOOD AND SIDE EFFECTS: A seven (7) point scale was used to measure changes in patient mood states: Depression and Anxiety. For example, the scale for depression ranged from −3, “much less depressed”, to +3, “much more depressed”, with 0 indicating no change. A psychiatrist asked the patient to rate their mood immediately following each sonication. Additionally, the patient filled out the Generic Assessment of Side Effects (GASE) questionnaire prior to enrolling in the trial and at the end of each sonication session. The patient was asked to rate each of 36 different symptoms from 0—not present, 1—mild, 2—moderate, 3—severe, and note if they found this symptom related to the treatment.

STUDY PROTOCOL: In study S1, the first patient visit took place inside an MRI suite (Magnetom VIDA, 3 T, Siemens). Prior to the imaging, a mask 700a comprising a thermoplastic mask was molded onto the patient's face, and treatment device 100 (including treatment assembly 150a and 150b as described herein) was coupled to the patient's head. Structural MRI images were recorded for the previously described registration. The second visit comprised an MRI-free visit in which the patient's head was immobilized in the same type of mask 700a comprising a thermoplastic mask, and the treatment assemblies 150a and 150b of the treatment device 100 were locked in the same position as during the initial visit. Ultrasonic stimulation was delivered into the subgenual cingulate cortex and the ventral striatum. The target locations of the brain were presented randomly, in a blinded manner, and were interleaved with the above described active sham stimulation (plane waves) over a 1.5 h neuromodulation session.

MRI: Imaging data was recorded with a 3 T MRI scanner (MagnetomVida, Siemens). A Siemens flex coil (size: small) was placed over the anterior and superior aspects of the patient's head to maximize the signal-to-noise ratio of the acquired signals.

ANATOMICAL ACQUISITION: Anatomical images were collected using a magnetization prepared radio frequency pulses and rapid gradient echo (MPRAGE) sequence with an ascending, anterior to posterior acquisition of 192 sagittal slices with thickness of 1.3 mm. The repetition time (TR) was 2400 ms; echo time (TE) was 2.26 ms; inversion time (TI) was 1060 ms; and echo spacing was 6.84 ms. The field of view (FOV) was 256 mm, with bandwidth of 200 Hz/pixel and a flip angle of 8 degrees.

MEASUREMENT OF STEERING RANGE AND FOCAL VOLUME: Study S1 includes measurements of steering range and focal volume.

SKULLS: Four intact ex-vivo human skulls were used in study S1. A large opening was made at the bottom of each skull to enable field measurements inside the skull. Each skull was degassed at approximately −25 mmHg overnight in deionized water. Following the degassing, the skull was transferred, within the degassed water, into an experimental tank filled with continuously degassed water (AIMS III system with AQUAS-10 Water Conditioner, Onda).

STEERING RANGE: Beamforming range was measured inside four ex-vivo skulls with hydrophone field scans. At each location in the scans, transducers of USTs 155U were fired individually, and the received signals recorded. The total pressure at each point was taken as the sum of these individual waveforms under perfect focusing through the skull. Perfect focusing entails adjusting the delays on the received signals from each element such that they arrive at the hydrophone at the same time, perfectly in phase. Delays are found by maximizing the cross-correlation between all waveforms. The scans were performed over 2D planes in the XY, XZ, and YZ planes, with the span of the X (axial), Y (lateral), and Z (elevational) dimensions being 80 mm, 60 mm, 56 mm respectively, all with 1.5 mm step size. These 2D scans were then interpolated to a resolution of 0.2 mm and the full width half max (FWHM) distance in each dimension was calculated about the origin defined as the geometric center of the two arrays. In the X dimension, the FWHM value exceeded the scan bounds that were constrained to ±40 mm by the width of the ex-vivo skulls. To estimate the entire FWHM in this dimension, a 2nd-degree polynomial was fit to the measured field in the X dimension about the origin and extrapolated the field profile out to ±100 mm. The FWHM was calculated as the difference in the X coordinates where the extrapolated field profile first fell below 50% of the peak value.

PRESSURE FIELD HYDROPHONE SCANS: A capsule hydrophone (HGL-0200, Onda) secured to a 3-degree-of-freedom programmable translation system (Aims III, Onda) was used to record the ultrasound field emitted from each transducer of USTs 155U. The hydrophone used has a sensitivity of approximately-266 dB relative to 1V/μPa and aperture size of 200 μm. This aperture size is well within the ultrasound wavelength (2.3 mm) used. The 3D field measurements use a step size of 0.2 mm to provide high spatial resolution of each element's contribution to the total field. The hydrophone scans measured 40 mm by 40 mm planar scans in all three dimensions (XY, XZ, and YZ).

The scans were performed at the geometric center of the two ultrasound arrays in water and through an ex-vivo human skull. At each location in the scans, transducers of UST 155U were fired individually, and the received signals recorded. Since ultrasound pressure is additive, the total pressure was computed as the sum of the individual constituents.

FOCAL VOLUME: The focal volume of the arrays was quantified by measuring the total size of the intensity field above half the maximum value. Specifically, the convex hull of the voxels was used, just exceeding the half-maximum intensity in both the XY and XZ planes. For each position on the x-axis, a calculation was performed for the full width half max (the width of the focal volume at half-maximum intensity), in the Y and Z dimension. These products were then integrated over the x axis to get the total volume. The functions FWHM_{y}(X) and FWHM_{z}(X) denote the full width half max at position x in the Y and Z dimension, respectively. The focal volume then equals ∫FWHM_{y}(X) FWHM_{z}(X) dx.

MEASUREMENTS OF POSITIONING REPRODUCIBILITY: Session-to-session registration error was quantified across 5 human patients by measuring the position variability of both treatment device 100 and the patient. In particular, an optically tracked stylus (Brainsight, Rogue Resolutions Ltd.) was used to record the position of six fiducial markers (marker 115) of treatment assemblies 150a and 150b, as well as four anatomical landmarks on the patient (tip of the nose, left and right corners of the eye, and a mark on the left temple). These measurements were performed across 10 trials per patient. Before each session, system 10 (e.g., treatment device 100) and the patient were re-instantiated to simulate a new treatment session. After each session, the patient was taken out of mask 700a (e.g., a thermoplastic mask) and asked to stand up and walk away from the table used to perform the procedure. Treatment device 100 was unscrewed from a locked position and reset to a reference position. Each fiducial and anatomical landmark was measured three times with the stylus. The median of these three measurements was used as the fiducial's position.

The variability in position of treatment device 100 and patient was measured by calculating the mean location of each fiducial marker (markers 95 and 115) for each patient across all trials, and subtracting that number from the fiducial positions. Next, the combined variability of the patient and device 100 position was calculated by taking the difference of each fiducial (each marker 115) on device 100 from each fiducial (e.g. marker 95) on the patient. The deviation from the mean in these distances was measured for each pair across trials. The targeting accuracy constituted the average relative position variability across all fiducial pairs.

RESULTS: System 10 of the present inventive concepts has been configured to neuromodulate specific deep brain regions in a mammalian patient. In study S1, treatment device 100 comprises two treatment assemblies, treatment assembly 150a and 150b, each including a phased array of transducers UST 155U, the assemblies 150 positioned at opposite sides of the head of the patient (e.g. as shown in FIG. 2A). The dual phased arrays enable device 100 to electronically focus ultrasound into specified deep brain targets and deliver ultrasound specifically through the relatively acoustically permissive areas of the skull: the parietal and temporal bone.

Prior to an application, the patient's head is immobilized using a mask 700a comprising an individually fitted, standard radiological mask as shown in FIG. 2B. Lateral windows in the mask 700a enable the ultrasound to propagate into the head using a coupling medium (e.g., couplers 156, coupling gel 700b, or both). An imaging device 800 comprising an MRI provides the means for accurate registration of treatment device 100 with respect to the patient's brain anatomy. Mask 700a is attached to the same housing 110 as that which holds the treatment assemblies 150a and 150b, thus the MRI-based registration procedure only requires one MRI scan. Subsequent treatments (e.g., ultrasound energy deliveries to the patient's brain) using treatment device 100 can be reproducibly performed without an MRI.

FIGS. 2C-D illustrate the intensity fields produced by treatment device 100, overlaid on a patient's brain anatomy. For the specified target of the subgenual cingulate cortex (SGC), the intensity field had lateral×elevational×axial dimensions of 2.4 mm by 3.6 mm by 20.4 mm (y, z, and x dimensions of the Montreal Neurological Institute coordinate system). The total field volume of 0.142 cm³ was equivalent to a sphere with a radius of 3.24 mm.

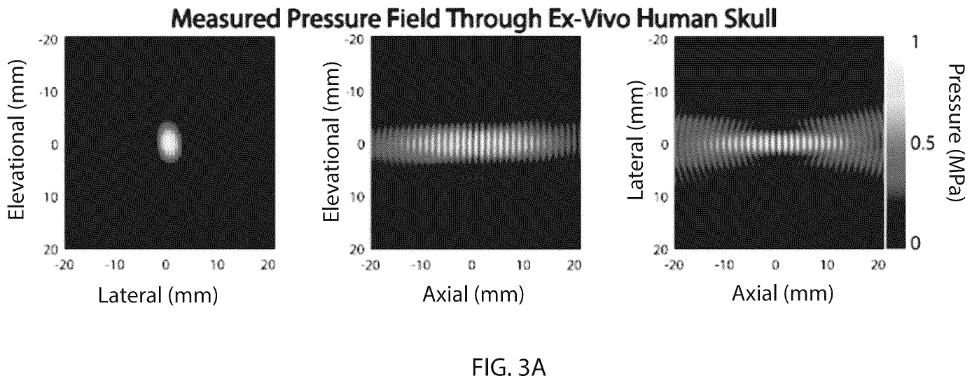

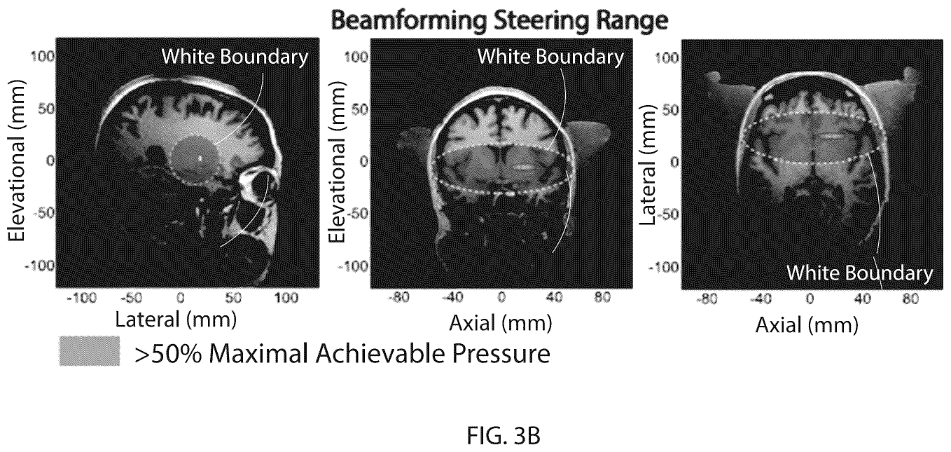

The phased-array configuration of treatment device 100 enables the focusing of the ultrasound energy into specified targets electronically, without moving the patient or the device 100. In FIG. 3A, an example pressure field measured in three dimensions through an ex-vivo human skull is illustrated. The middle and right panels show that the two arrays of ultrasound transducers UST 155U of treatment assemblies 150 produce a notable standing wave, as expected. In FIG. 3B, electronic targeting range (tissue within the dashed lines) is measured through four ex-vivo skulls. The white boundaries outline the regions in which treatment device 100 can deliver 50% of its maximal pressure output. The fields were measured inside an ex-vivo human skull and overlaid on an anatomical MRI for comparison. Any target within this region can be reached within a few dozens of microseconds. Treatment device 100 is configured to allow steering of the focal point through beamforming to modulate single or multiple targets within a relatively broad treatment envelope, as shown in FIG. 3B. The addressable space within which treatment device 100 delivers at least half of its maximal pressure, measured using hydrophone inside four ex-vivo skulls, spans 110.8 mm±5.69 mm, 46.1 mm±3.4 mm, 44.8 mm±2.7 mm (mean±s.d.) in the axial, lateral, and elevational dimensions, respectively (FIG. 3B). To target additional parts of the brain, housing 110 is adjustably attached to treatment assemblies 150a and 150b, such that these assemblies 150 can be physically translated in the patient's anterior-posterior and superior-inferior dimensions to target particular regions of interest.

The registration error was evaluated between the patient's head and the treatment assemblies 150 in five patients across ten sessions. Across all sessions (n=10 in each patient), the position of ultrasound transducers UST 155U was displaced from their initial position on average by 0.89 mm±0.64 mm (mean±s.d.).

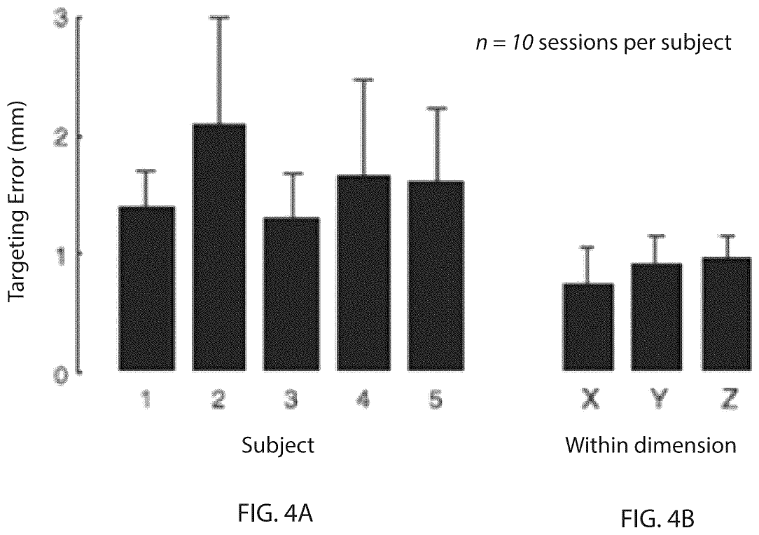

Within each dimension (x, y, z), the transducers USD 155U were displaced by 0.45 mm±0.32 mm, 0.43 mm±0.14 mm, and 0.44 mm±0.17 mm. The error in the head positioning was also within the acceptable range. Using fiducial markers positioned on the subjects' head (marker 95), an average error was detected of 1.28 mm±0.66 mm, and 0.53 mm±0.19 mm, 0.68 mm±0.27 mm, and 0.71 mm±0.31 mm in the x, y, and z dimensions, across all subjects and sessions. The relative error was computed between the transducers USD 155U and the patient's head, which is the ultimate metric that informs on the targeting accuracy. The relative error was 1.64 mm±0.66 mm across subjects and 0.77 mm±0.50 mm, 0.93 mm±0.41 mm, and 0.99 mm±0.49 mm in the x, y, and z dimensions, as shown in FIG. 4A-B. FIG. 4A is a bar chart illustrating the mean±standard deviation difference between the relative distance of the transducers UST 155U and markers 95 placed on the heads of the five patients described immediately hereabove, and FIG. 4B is a bar chart of the difference separately for each of the dimension averages over the 5 patients.

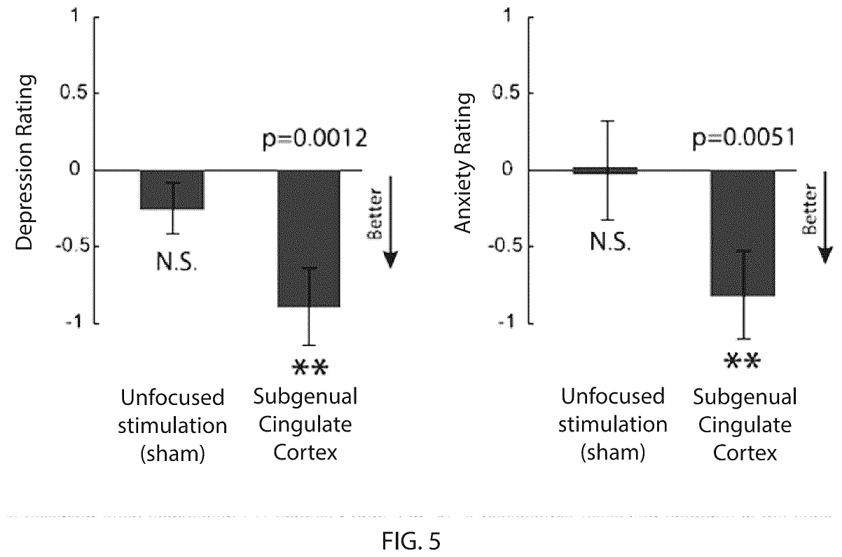

Treatment device 100 was used to modulate the SGC in two patients, patients PS1-1 and PS1-2, each with treatment-resistant depression (trial NCT05301036). Major depression is commonly associated with hyperactive SGC, and treatment device 100 and other components of system 10 can be used to provide a transient suppression of the SGC in order to improve subjective mood states. To suppress neural activity, ultrasound was delivered by treatment device 100 into the brain target at a low duty cycle value, which tends to inhibit neuronal activity. The modulation of the SGC had a positive effect on metrics of depression and anxiety in both patients. FIG. 5 includes two bar charts that illustrate this positive effect. Combined self-reported mood scores across patients following each stimulation procedure are shown. A psychiatrist assessed changes in the patient's mood immediately following each ultrasound delivery procedure, measuring changes to depression on a seven-point scale from −3 (indicating much less depressed) to +3 (indicating much more depressed), with 0 representing no change. Sham stimulation delivered into the brain had the same energy and waveform but was not focused. The use of the sham controls for potential generic auditory and tactile artifacts that could be associated with a transcranial ultrasound treatment. Patients were blinded to the individual stimuli. There were positive effects on mood following the modulation of the SGC for at least 60 s, but not in other cases including sham stimulation. Stimulation of the SGC for duration 60 seconds and longer led to significant improvements in depression (t₁₈=3.54, p=0.0012, two-tailed t-test) and anxiety (t₁₈=2.87, p=0.0051, two-tailed t-test) metrics. Sham stimulation caused no significant effects on depression (t₇=1.53, p=0.17) or anxiety (t₇=0, p=1) metrics.

The safety of the stimulation at the behavioral and anatomical levels were evaluated. At the behavioral level, patients completed a standard clinical side effect questionnaire. No adverse effects were noted by either patient or the attending psychiatrist. At the anatomical level, structural T1-w and T2-w MRIs of the brain were collected. No apparent changes were evident.

DISCUSSION: System 10 is configured to: minimize the distortions that ultrasound experiences when propagating through the skull; provide flexible electronic targeting of deep brain regions; and use mechanical registration for practical, low-cost registration inside and outside of an MRI.

System 10, via housing 110 and other components of system 10, can be configured to deliver energy, such as ultrasound energy, through the temporal-parietal windows of the skull, to enhance the delivery of energy into the brain of a patient. System 10 can be further configured to correct for all aberrations of energy delivery by the skull, and other obstacles, as described herein. All energy-delivering transducers of treatment assembly 150 (e.g. phased arrays of ultrasound transducers USTs 155U) are independently controllable. In this configuration, it is possible to adjust the emission time and amplitude separately for each energy delivery element of treatment assembly 150, thus optimizing the energy delivery.

The phased arrays of energy delivery elements 155 of treatment assembly 150 provide the capacity to target many regions within the deep brain. For instance, the modulation of the SGC in a patient, patient S1A, required steering of the focus 17 mm laterally, and 9 mm elevationally, from the geometric center of the arrays. The electronic beamforming also enabled ultrasound delivery into the superior and inferior sections of the subgenual cingulate cortex, as well as the ventral striatum. The capacity of treatment device 100 to electronically steer the delivery of ultrasound into different targets rapidly (e.g., in less than 1 millisecond), enables rapid stimulation sequences that modulate neural networks in precise spatiotemporal patterns. This capacity provides effective treatments of mental and neurological disorders, which require high targeting precision as well as flexibility in targeting distant nodes of neural networks.

Repositioning of the patient's head and the transducers of treatment assembly 150, based on a single registration procedure using MRI, was found to be reproducible from session to session, with a relative positioning error between markers 95 on the patient and markers 115 on treatment device 100 of 1.64 mm on average (reference FIGS. 4A-B). The mechanical registration method allows for accurate targeting of deep brain areas inside and outside of an MRI scanner. For MRI-free operations, mask 700a (e.g. a thermoplastic mask) provides a convenient approach to mitigate costs, such as the costs associated with optical neuro-navigation systems.

System 10 can be used to complete various medical procedures, such as diagnostic procedures, therapeutic procedures, or both. The ability to flexibly modulate specified deep brain targets provides a unique tool to guide invasive approaches such as deep brain stimulation or ablative brain treatments. By precisely modulating candidate targets in sequence, system 10 can be used to determine the brain region that maximizes the sign or symptom improvement in each individual patient. This region can constitute the target for a subsequent invasive treatment. The flexible neuromodulation provided by system 10 also provides a unique tool for manipulation of deep brain structures, which can further our causal understanding of human brain function. By systematically modulating specific brain regions, operators using system 10 are equipped to determine how these regions are causally involved in given behaviors.

System 10 can be configured to apply low-intensity ultrasound to one or more targets within the brain, for a sufficient amount of time (e.g., about 150 s), to induce durable effects within the stimulated structures. These neuroplastic effects are believed to be mediated, at least in part, by activation of glial cells and the ensuing effects on synaptic processes. These effects provide a unique opportunity for durable circuit reset, akin to electroconvulsive therapy or repeated applications of TMS, but now in a much more targeted manner using system 10. The targeted nature of the stimulation is expected to increase treatment effectiveness and safety and provide a treatment option to patients for whom the current approaches are inadequate.

### Study S2

In one study, study S2, a thirty (30) year-old Caucasian woman, patient PS2-1 with severe treatment-resistant non-psychotic depression was recruited into study S2. The patient had a history of electroconvulsive therapy with full remission but without sustained benefit. Magnetic resonance imaging was used to co-register treatment device 100 to the patient's brain anatomy and to evaluate neural responses to stimulation using system 10 of the present inventive concepts. Brief, 30-millisecond pulses of low-intensity ultrasound every 4 seconds that was delivered into a target region comprising the subcallosal cingulate cortex (SCC) of the patient caused a robust decrease in functional magnetic resonance imaging blood oxygen-level-dependent activity within the target region. Following repeated stimulation of three anterior cingulate targets, the patient's depressive symptoms resolved within 24 hours of the stimulation. The patient remained in remission for at least 44 days afterwards.

System 10 of the present inventive concepts is configured to overcome various limitations of previous stimulation approaches. System 10 is configured to directly measure and compensate for the ultrasound attenuation caused by the head, hair, and other obstacles as described herein, such as to safely and effectively deliver deterministic ultrasound intensity into one or more deep brain targets. Treatment device 100 can be applied, under MRI guidance, to the SCC and associated circuits in a patient with intractable depression. Significantly, a single session of ultrasound stimulation of three SCC-associated targets led to rapid remission of the depressive symptoms in patient PS2-1. Patient PS2-1 remained in remission at the last assessment 44 days following the treatment.

CASE PRESENTATION: Patient PS2-1 is a 30-year-old Caucasian female with severe treatment-resistant depression. The diagnosis was established as a failure to respond to two or more adequate first-line medication treatments. Diagnosis of recurrent major depressive disorder was confirmed with the Mini International Neuropsychiatric Interview (MINI) structured interview (7.0.0). There is a family history of mood disorders, including major depressive disorder, bipolar disorder, and suicide. Onset of depression and anxiety were noted at the age of 13. Between ages 14 and 29 years, patient PS2-1 was treated with psychotherapy and underwent medication trials of sertraline, bupropion, citalopram, fluoxetine, duloxetine, trazodone, aripiprazole, quetiapine, clonazepam, lorazepam, lamotrigine, and lithium. Patient PS2-1 reported initial benefit from most of these agents but less benefit over time; fluoxetine in particular was associated with marked increase of suicidal ideation, which led to her first psychiatric hospitalization. Patient PS2-1 experienced peripartum worsening of depression associated with two live births and one miscarriage. Patient PS2-1 was hospitalized three times for suicidal ideation. There is no history of attempted suicide, mania, substance use disorder, or psychosis, and no notable medical comorbidities. Patient PS2-1's depressive episode reached heightened severity at age 29 [Quick Inventory of Depressive Symptoms selfreport (QIDS-SR) score of 16, severe]. Patient PS2-1 underwent a course of bifrontal electroconvulsive therapy (ECT) and experienced significant improvement with an acute series of eight sessions: QIDS-SR score decreased to 4 (remission) 1 week after the acute series. Patient PS2-1 had 30 ECT maintenance sessions over the following year. Attempts to reduce the frequency of treatments resulted in recurrence of symptoms. ECT was discontinued due to cognitive and memory problems. At this time, patient PS2-1 was evaluated and enrolled in study S2 with a six-item Hamilton Depression Rating Scale (HDRS-6) score of 11 and QIDS-SR score of 16. At the time of enrollment and throughout the study, patient PS2-1 was managed on a combination of bupropion extended release (XL) 450 mg daily, duloxetine 90 mg daily, and lithium extended release (ER) 450 mg twice daily. No changes were made to the patient's medication regimen during the study. There was no evidence of a developmental or cognitive disorder.

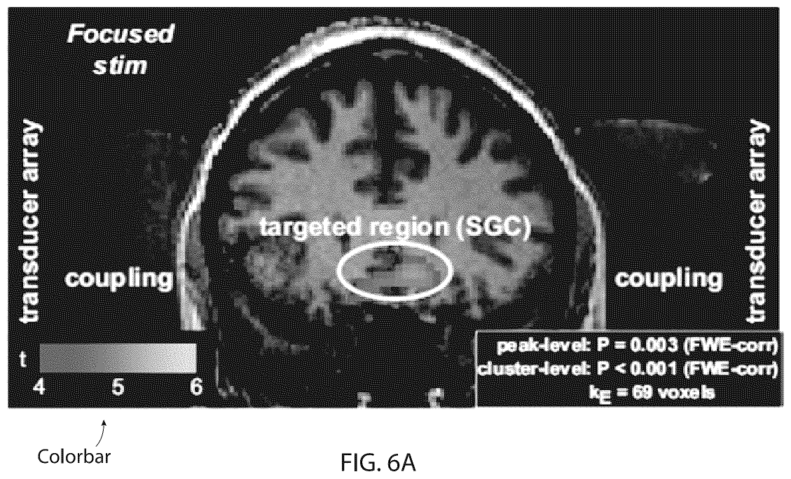

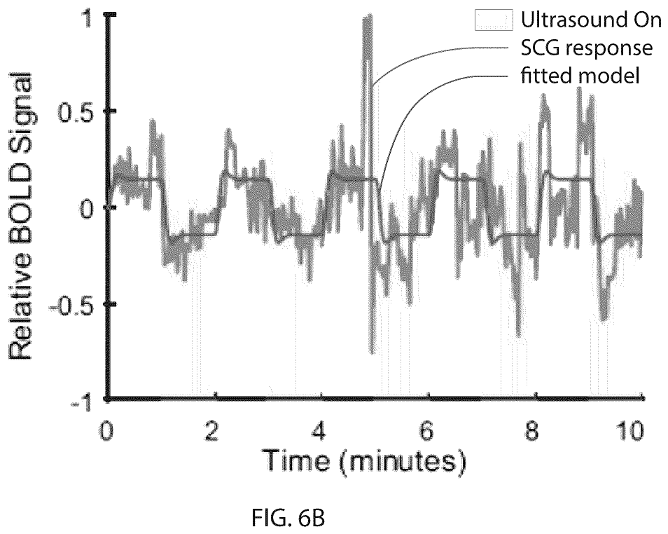

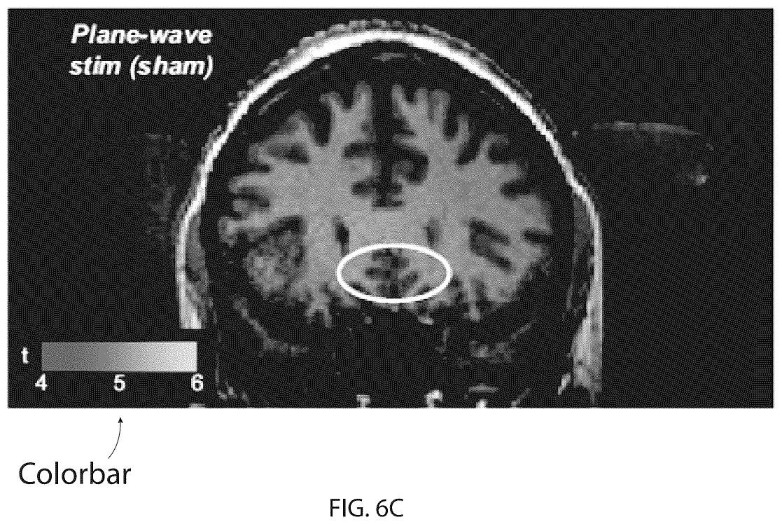

During the registration and subsequent therapy procedures (e.g., as described herein), treatment device 100 was applied to the patient as shown in FIG. 2B, wherein a mask 700a comprising a radiological mask with lateral windows secured the patient relative to treatment device 100, and couplers 156a and 156b. A compensation procedure was performed (also as described herein), and therapeutic ultrasound delivery via a treatment device 100 was performed using a compensated drive signal. The treatment device 100 used in study S2 comprised a first treatment assembly 150a comprising 126 USTs 155U and a second treatment assembly 150b also comprising 126 USTs 155U. The engagement of the modulated target, SCC, was validated with functional MRI (as reference FIGS. 6A-C). A standard Siemens flex coil was positioned over the patient (0.8% duty). The on and off periods were presented in 1 minute ON blocks of ultrasound delivery, followed by 1 minute OFF blocks of no ultrasound, for a total of up to 10 minutes. The MRI scanner (an imaging device 800 comprising an MRI) acquired fMRI BOLD signals during the stimulation. In FIG. 6A, the colorbar shows the t-statistic associated with the BOLD difference between the ON and OFF blocks. The white circles outline the approximate location of the SCC. In FIG. 6B, the modulation hemodynamic response is illustrated. In FIG. 6C, a control stimulation is illustrated, the control used to control for potential generic artifacts associated with ultrasound, where a stimulus was delivered that had the same waveform and pressure amplitude as the stimulus focused into the SCC, but was unfocused (e.g., transducers UST 155U emitted a plane wave).

The positioning of treatment assemblies 150a and 150b on opposite of the patient's head enables electronic focusing of the ultrasound into specified deep brain targets, as well as compensation (e.g., via the compensation method described herein) that adjusts for the ultrasound attenuation by the skull, hair, coupling media, and other obstacles.

In order to evaluate the immediate effects of the stimulation on mood states, system 10 was used to modulate three separate areas of the cingulate cortex in patient PS2-1 over a 2-hour stimulation session. The intensity field delivered into the brain had lateral by elevational by axial dimensions of 2.4 mm by 3.6 mm by 20.4 mm (y, z, and x dimensions of the Montreal Neurological Institute (MNI) coordinate system). The targets were centered on posterior SCC [MNI coordinate (0, 26.21, −8.11) (x, y, z from MNI center coordinate)], anterior SCC [MNI coordinate (0, 34.21, −6.11)], and pregenual cingulate [MNI coordinate (0, 34.21, 3.11)]. These targets were chosen to maximize the probability of modulating white matter tracts within the SCC. Each target was sonicated with a 650 kHz continuous wave for 30-millisecond ON periods followed by 4-second OFF periods (0.8% duty) for an average duration of 2 minutes (range 20-180 seconds). The estimated peak pressure at target was 1.0 MPa following a compensation (performed by system 10) for the ultrasound attenuation by the head, hair, and other obstacles. Each target was sonicated ten times, with randomized order between the three sites, for a total of 30 stimulation epochs spanning 64 minutes of active stimulation. The stimulation intensity was maintained below the US Food and Drug Administration (FDA) 510(k) Track 3 guidelines (peak intensity less than 190 W/cm² and time-averaged intensity less than 720 mW/cm²).

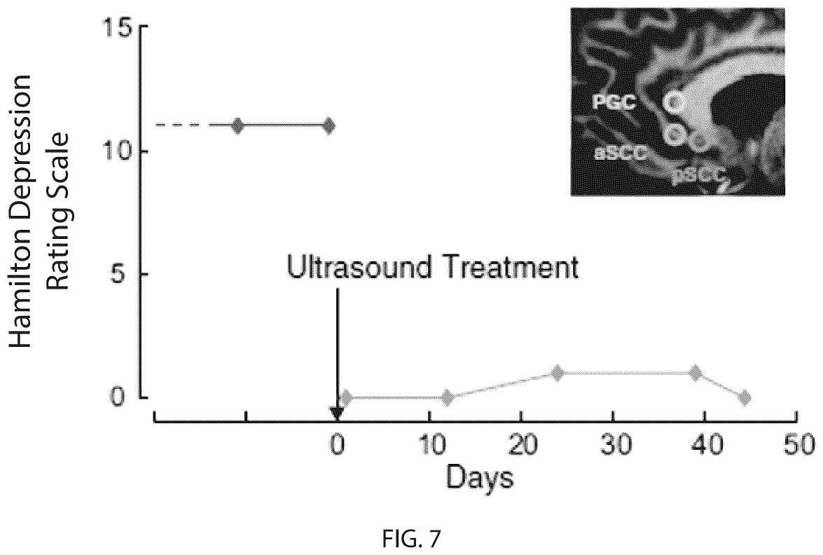

To evaluate any durable effects of the stimulation, HDRS-6 scores were collected before and after the stimulation. FIG. 7 illustrates that the noninvasive deep brain stimulation provided by system 10 can improve mood states in a patient with major depression. Following a single treatment session of 64 minutes of active stimulation to three separate targets of the SCC, patient PS2-1's HDRS-6 score fell from 11 to 0, as shown in FIG. 7, indicating effective remission. On the day following treatment, patient PS2-1 reported, “This is the first time in three years I have felt like myself; it feels like my brain has been woken up.” The effects were durable and the patient remained in remission (HDRS-6=0) for at least 44 days following the sonication, the last assessed timepoint. About 5 months after the stimulation, she started to notice a recurrence of the depression; medications were continued unchanged during the 5 month interval.

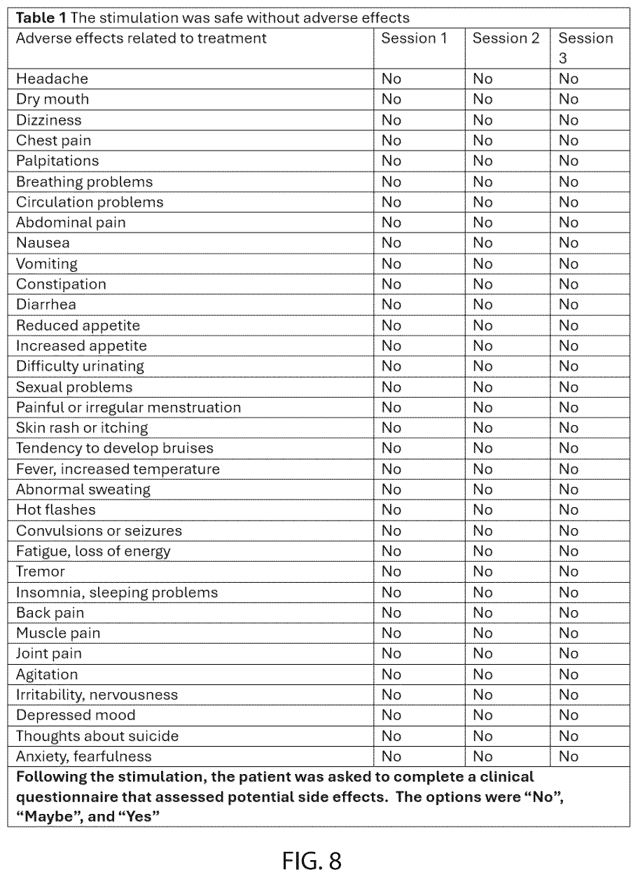

In Study S2, the safety of the stimulation provided by system 10, at the behavioral and anatomical levels, was evaluated. At the behavioral level, patient PS2-1 completed a standard clinical questionnaire of stimulation side effects. No adverse effects were noted by the patient nor the attending psychiatrist. Patient PS2-1 completed the General Assessment of Side Effects (GASE) survey and reported no side effects related to treatment (reference the table of FIG. 8). Moreover, no anomalies were observed on either T1-weighted or T2-weighted MRIs of the subject's brain.

DISCUSSION AND CONCLUSIONS: Study S2 demonstrated rapid and sustained improvement in depression following direct ultrasonic modulation of deep brain targets associated with the SCC using system 10. The stimulation was followed by remission lasting for at least 6 weeks. No safety concerns or side effects were noted. The approach provides three notable strengths over existing neuromodulation devices in that it (1) delivers stimulation noninvasively into deep brain targets, (2) provides precise and flexible electronic targeting, and (3) delivers controlled stimulation intensity into the targets.

Using functional MRI (fMRI), it was demonstrated that treatment device 100 significantly and substantially engaged the specified deep brain target, the SCC, and its associated circuits. The stimulation resulted in a significant decrease in fMRI BOLD activity at the target, which suggests an inhibition of the SCC. This effect was only observed during active stimulation and not during sham stimulation.

Therapies provided using system 10 are not limited to modulation of the SCC; the ultrasonic array configuration of treatment device 100 is capable of modulating targets throughout the deep brain. For instance, device 100 can deliver stimulating energy to the ventral posteromedial or ventral posterolateral nuclei of the thalamus, such as to treat patients with chronic pain.

Transcranial low-intensity ultrasound has been safely applied to human subjects in previous studies, but the strongly aberrating properties of the skull have severely limited the predictability of the delivered intensity. Uncertainty associated with existing transcranial ultrasound delivery could raise safety concerns, since an overcompensation for the ultrasound attenuation of the skull could lead to mechanical or thermal tissue damage. Treatment device 100 and the other components of system 10 measure the acoustic properties of a patient's skull, hair, and other obstacles, using a through-transmit scan to determine adjustment parameters to be used in the therapeutic ultrasound delivery, in order to effectively deliver stimulation while remaining within well-established safety limits. The ultrasound intensity delivered in study S2 is limited to the FDA 510(k) Track 3 safety guidelines for safe ultrasound imaging: the spatial peak temporal average intensity of less than 0.72 W/cm², and the spatial peak pulse average intensity less than 190 W/cm².

System 10 provides ultrasound of sufficient duration and intensity induces durable neuroplastic effects in the target circuits. These effects are believed to be mediated, at least in part, by activation of glial cells and the ensuing effects on synaptic processes. This and related molecular pathways provide unique opportunities for durable circuit reset, akin to electroconvulsive therapy or repeated applications of transcranial magnetic stimulation, but now applied in a targeted manner and directly to the involved deep brain circuits. This approach will increase the effectiveness and safety of neuromodulation treatments, providing targeted patient-specific reset of the malfunctioning deep brain circuits.

As demonstrated in clinical studies, treatment device 100 and other system 10 components can be configured (e.g., collectively configured) to treat a patient suffering from depression. This includes patients suffering from major depression, and possibly depression comorbid with anxiety, chronic pain, Alzheimer's disease, and/or post-traumatic stress disorder.

### Study S3

System 10 was used in another study, study S3 described herein, in which delivery by system 10 of combined ultrasound and magnetic fields were performed to stimulate peripheral nerves of a mammalian patient. As used herein, “Lfield” refers to the resulting electric field, and “Lstim” refers to the resulting stimulatory effects, given their origin in the Lorentz equation and their electrical and local nature.

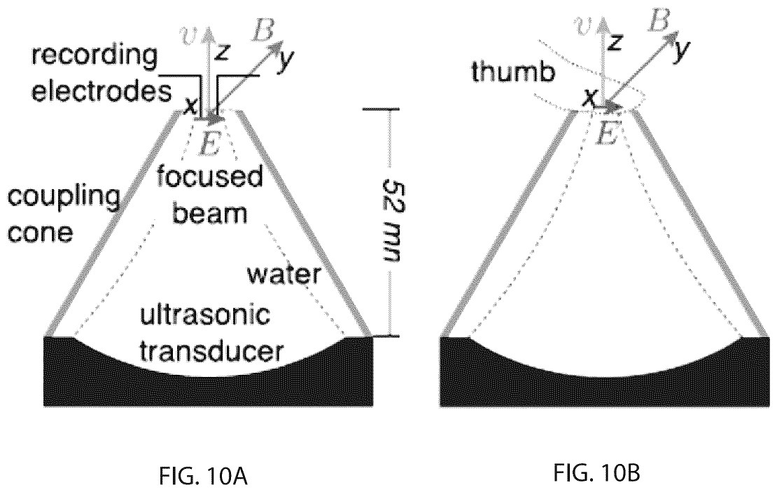

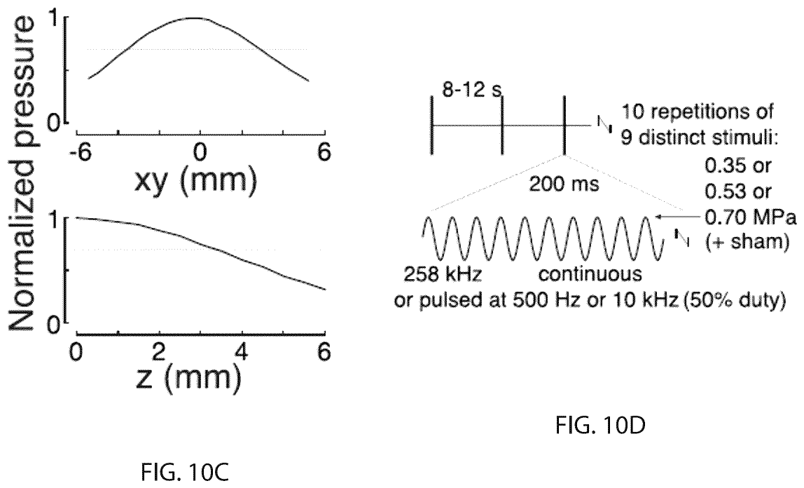

ULTRASONIC APPARATUS: The ultrasonic stimuli for the field assessment and the peripheral nerve stimulation were generated using a focused, MRI-compatible ultrasonic transducer with a 64 mm diameter and 52 mm focal depth. The transducer was operated at 258 KHz. A water-filled coupling cone of 1 mm-thick plastic was used to focus the ultrasound into the target (reference FIG. 10). The height of the cone was 52 mm and its diameter 70 mm. The cone's aperture had a 16 mm diameter at the ultrasound target. System 10 generated stimuli that produced the stimulation waveforms in a programmable function generator portion of system 10. The signals were amplified using a 55 dB, 250 kHz-30 MHz power amplifier of system 10.

MEASUREMENT OF THE ULTRASONIC FIELDS: The pressure fields were measured in free field, in a water tank, at the location of the ultrasound target. The pressures were measured using a capsule hydrophone (HGL-0200, Onda). The hydrophone was calibrated between 250 kHz and 40 MHz and secured to 3-degree-of-freedom programmable translation system (Aims III, Onda). The spatial distribution of the generated ultrasound pressures is shown in FIG. 10C, a peak-normalized ultrasound pressure field. The pressure profile was averaged over the x and y dimensions. The dotted lines show the 0.707 (0.5) pressure (intensity) levels to characterize the fields using full-width-at-half-maximum values. The full-width-at-half-maximum (FWHM) diameter was 6.5 mm in the x-y dimension, and focal length (z-dimension) 3.3 mm. The hydrophone measurements incur about 1 dB error (HGL-0200, Onda). This can introduce a discrepancy between the theoretical and measured fields (reference FIG. 9B).

MAGNETIC FIELD: The peripheral nerve stimulation performed by system 10, and the associated measurements, were performed inside a 7 T MRI scanner (Bruker BioSpec). The ultrasound transducer assembly of system 10 was positioned inside the bore at a distance 20 cm from the exit plane of the bore. The static magnetic field inside the bore is considered relatively uniform. The magnetic field pointed in the direction perpendicular to the ultrasonic field (reference FIG. 10).

MEASUREMENTS OF THE GENERATED ELECTRIC FIELDS: The electric fields generated by system 10 were measured using a pair of copper electrodes positioned at the ultrasound target (reference FIG. 10A). The measurements were performed in the indicated geometry and followed rotation of the setup 90 degrees with respect to the magnetic field. The inter-electrode distance was 3 mm. The coupling cone was filled with saline. The electrodes were insulated with only their tip exposed to the medium. 100 repetitions of 50 ms continuous, 258 kHz tone burst at pressures amplitudes of 0.1 MPa, 0.3 MPa, and 0.5 MPa were collected. For each repetition, the peak amplitude of the voltage elicited between the electrodes was measured, and the 100 values for each pressure was averaged together. By contrasting the effects of the default geometry Lfield (ref FIG. 10A) with that rotated 90°, and subtracting the respective voltage amplitudes, the measurements were not influenced by potential artifacts associated with the ultrasound.

COMPUTATION OF THE GENERATED ELECTRIC FIELDS: The generated electric fields obey the Lorentz equation,

$$E = \frac{PB}{Z} .$$

In this equation, the acoustic impedance of Z=1.58 MRayl was used. For the measurements to be deterministic, the electrodes were inserted into the saline at a depth of 1.5 mm. This depth corresponds to one-quarter wavelength. This depth was used such that the electrode tips were positioned at a defined location within the antinode of the wave reflected from the water-air interface. The antinode experiences double the pressure, thus leading to

$$E = \frac{2 PB}{Z}$$

(reference FIG. 9B).

NERVE STIMULATION: In study S3, eighteen human subjects (6 females, 12 males, aged between 21-38 years) participated. Subjects were asked to gently rest the thumb of their right hand on the plastic coupling cone which was filled with degassed water (ref. FIG. 10B). Subjects had their eyes closed and wore noise-cancelling earmuffs (X4A, 3 M; 27 dB noise reduction) so that they could fully focus on the stimuli. Subjects could not hear or see the stimuli or their generation.

STIMULI: The stimulation was performed inside the bore of the 7 T MRI scanner or at a 3 m distance away from it. The stimulation order was randomized, without replacement, such that half of the subjects experienced the stimulation in the scanner first and the other half outside of the scanner first. Subjects were asked to place the finger on the aperture in the direction perpendicular to the ultrasonic and magnetic fields (reference FIG. 10B) to maximize the Lstim effects.

In study S3, nine distinct stimuli were used, comprising three pressure levels and three distinct waveforms (reference FIG. 10D). A tenth, sham stimulus, delivered negligible pressure (5 kPa, corresponding to the noise level of the amplifier-transducer output) under the same conditions. The parameters were chosen to provide safe and effective stimulation. The system 10 transducer's fundamental carrier frequency was 258 kHz. The duration of each stimulus (200 ms) was chosen to provide ample time for potential integrative effects. The peak pressure amplitudes of the ultrasound measured at the center of the aperture were 0.35 MPa, 0.53 MPa, and 0.7 MPa. The peak pressures were chosen such as to trigger appreciable electric intensities at target (up to 3.1V/m), but low enough to comply with the I_{SPPA} Track 3 510(k) recommendation for each pulse and within the I_{SPTA} recommendation over the course of the study, and low enough to prevent unpleasant nociceptive responses. The stimuli were either continuous (200 ms of tone burst) or pulsed at 500 Hz or 10 kHz, both at 50% duty cycle. The pulsed stimuli were added under the hypothesis that pulsed stimuli may provide multiple onset responses, thus amplifying the stimulation. The effect of Lstim was observed regardless of whether the stimulus was continuous or pulsed; there was only a weak (reference table 1) interaction of the stimulus waveform and magnetic field.

TABLE 1

| | Nociceptive | Tactile |
|---|---|---|
| M | <0.001 | 0.0042 |
| P | <0.001 | <0.001 |
| W | <0.001 | <0.001 |
| M × P | 0.0012 | 0.23 |
| M × W | 0.043 | 0.040 |
| P × W | <0.001 | <0.001 |
| M × P × W | 0.54 | 0.83 |

In Table 1, the effects of magnetic field (M), ultrasound pressure (P), and stimulus waveform (W; continuous or pulsed) on the frequency of nociceptive (left column) and tactile responses (right column) are shown. These effects were accessed using a three-way ANOVA that featured the three main effects and all possible interactions. Bold entries are significant (p<0.05).

In study S3, there were ten repetitions of the ten stimuli, producing a total of 100 stimulation trials per subject inside the scanner and 100 trials outside the scanner. The stimuli were delivered every 8-12 s. The stimuli were drawn from the 100-stimulus set randomly without replacement. This way, stimulus order could not affect the results.

RESPONSES AND THEIR ASSESSMENT: Subjects were instructed to report a percept with a verbal command of any combination of: Pain, Vibration, Tap, and their intensity: 1: low, 2: medium, 3: high. Following each stimulus, a study 3 data collector was prompted to enter the reported sensation (or lack thereof) and its intensity into a data logging program. The data collector was blinded to the stimuli. Following the experiment, for each stimulus type, the response magnitude was computed as the proportion of trials in which subjects' registered a response, weighted by the reported intensity. The principal results were the same regardless of whether the percepts were weighted by their intensity or were considered binary (reference Results section hereinbelow). Vibration and tap responses were grouped together as tactile.

ACOUSTIC CONTINUUM: The acoustic impedance of water and skin, including soft tissues, are closely matched (1.48 MRayl compared to 1.68 MRayl). This way, about 99.6% of the energy,

$$1 - {R}^{2} = 1 - {( \frac{1.68 - 1.48}{1.68 + 1.48} )}^{2} ,$$

was delivered into the finger. The water-finger interface is therefore essentially acoustically transparent and can be considered as a continuum from the perspective of ultrasound.

STIMULUS SAFETY: The ultrasonic stimuli used in this study were safely below the FDA 510(k) Track 3 recommendations (FDA 2023). In particular, the highest peak pressure used in the study, 0.7 MPa, corresponds to peak intensity of 15.3 W/cm², which is well below the FDA recommendation of I_{SPPA}=190 W/cm² (reference Table 2). In addition, the time-average spatial peak intensity was I_{SPTA}=150 mW/cm², also below the FDA recommendation of I_{SPTA}=720 mW/cm². The computation of the charge density (reference Table 2) assumed brain conductivity of 0.26 S/m. Thus, stimuli of much higher levels could be used, from both the ultrasound safety and electrical stimulation safety perspectives. The 0.7 MPa maximum allowed all sensations to be tolerated by the subjects. The thumb function was normal following the experiments and its sensation remained unaffected in all subjects.

TABLE 2

| | | | | | | | Charge |
|---|---|---|---|---|---|---|---|
| Pressure | E | | On | Off | [symbol] | [symbol] | density |
| (MPa) | (V m⁻¹) | Waveform | (ms) | (ms) | (W cm⁻²) | (W cm⁻²) | (μC cm⁻²) |
| 0.35 | 1.53 | Pulsed | 100 | 9900 | 3.8 | 0.0 | 1.4 |
| 0.53 | 2.30 | Pulsed | 100 | 9900 | 8.6 | 0.0 | 2.1 |
| 0.70 | 3.06 | Pulsed | 100 | 9900 | 15.3 | 0.1 | 2.8 |
| 0.35 | 1.53 | Continuous | 200 | 9800 | 3.8 | 0.0 | 2.8 |
| 0.53 | 2.30 | Continuous | 200 | 9800 | 8.6 | 0.1 | 4.2 |
| 0.70 | 3.06 | Continuous | 200 | 9800 | 15.3 | 0.2 | 5.6 |
| | | Safety guidelines | | | 190 | 0.72 | 30 |

[symbol]

indicates data missing or illegible when filed

Table 2 illustrates compliance with safety indices. The study used nine distinct stimuli: three levels of pressure and three distinct waveforms, one continuous (100% duty cycle) and two pulsed (both 50% duty cycle). All stimuli were 200 ms in duration and were delivered every 10 s on average. E is the induced peak Lstim intensity in a 7 T magnetic field. The computation of the charge density assumes brain conductivity of 0.26 S/m. Electrical stimulation should ideally not exceed charge density of 30 μC/cm². All stimuli are within the recommended safety levels. There were no detrimental acute or long-term effects reported by the subjects.

NON-HUMAN PRIMATE BRAIN STIMULATION: Two adult male rhesus non-human primates (Macaca mulatta, monkeys M1 and M2) participated in a brain stimulation portion of study S3. System 10 delivered ultrasound via a 256-element, MRI-compatible phased array of ultrasound transducers (e.g., USTs 155u). Briefly, the transducer array was inserted into a frame (e.g., housing 110) that was mounted into four titanium pins attached to the skull of the monkeys. This mounting system ensures reproducible targeting of energy delivery into the brain. Coupling to the head is mediated using a cryogel (e.g., coupling gel 700b). The coupling quality is validated prior to each session using an ultrasound imaging sequence. The monkeys were positioned inside MRI in a standard sphinx position. The monkeys were anesthetized with isoflurane (1.0%-1.25%+1-2 l min⁻¹ medical grade oxygen). The ultrasound was delivered by system 10 into two deep brain targets, the left and right lateral geniculate nucleus (LGN). Targeting of the LGN was validated using MRI thermometry. Ultrasound stimuli (100 ms duration, 480 kHz carrier frequency, 2 MPa amplitude) were applied to each LGN every 4 s in a strictly alternating manner (left LGN, right LGN, etc., every 4 s). The stimuli were either continuous or pulsed at 200 Hz pulse repetition frequency, 50% duty cycle. Data were collected for the monkeys fully positioned inside a 3 T MRI (Siemens TRIO and VIDA) or translated such that the head was positioned 2 m outside of the entry plane to the bore. The magnetic field at that distance comprised about 20 mT. Each monkey underwent two stimulation sessions. In monkey M1, the order of the pulsed stimulation was inside then outside for both sessions. For the CW sonications, the order in the first session was inside then outside, and the order was reversed in the second session. In monkey M2, the order of the pulsed stimulation was inside then outside for the first session and reversed for the second session. In monkey M2, only one CW session was recorded and the order of that session was outside then inside. There was at least a 2 minute interval between the inside and outside stimulation. Each session delivered 40 stimuli in total. This number was determined to provide sufficient statistical power while not imparting potentially detrimental effects on the stimulated tissue. A total of seven sessions were recorded (monkey M1: two sessions pulsed stimulation, two sessions continuous stimulation; monkey M2: two sessions pulsed stimulation, one session continuous stimulation), each contrasting the presence and absence of the magnetic field. The ultrasound pressure amplitude of 2.0 MPa, corresponding to I_{SPPA}=129.0 W/cm² was within the I_{SPPA}=190 W/cm² recommendation of the FDA 510k guidelines (FDA 2023).

The recordings and the quantification of gamma activity were analogous to a previous study (Webb et al 2023). The activity was assessed over 400 ms windows, overlapping every 100 ms. The gamma activity was normalized by the average gamma activity within a 1 s window preceding each stimulus, which provided a baseline for the assessment of the ultrasound and Lstim-evoked changes. Evoked activity was averaged over both posterior posts and over the left and right LGN stimuli.

RESULTS: The equation that governs the generation of electric field from ultrasonic and magnetic fields,

$$E = \frac{PB}{Z} ,$$

predicts that the generated electric field intensity should scale with the ultrasound pressure at the rate of 2 (reference FIG. 9B, solid line). In line with this prediction, a significant increase of the evoked electric intensity with ultrasound pressure was measured (reference FIG. 9B, dashed line; p=0.037, F-test of linear regression). The slope of the measured field, 5.5 V m⁻¹ 1 MPa⁻¹, was in good agreement with that computed using the Lorentz equation (4.4 V m⁻¹ MPa⁻¹) for the applicable magnetic field strength (7 T). Therefore, perpendicular applications of magnetic and ultrasonic fields indeed generate electric fields as predicted by the Lorentz equation, thus providing a direct validation of the concept.

FIGS. 9A and 9B illustrate that system 10 can produce Lfield intensities that are relevant to biological applications. A 0.5 MPa stimulus, well within the FDA 510(k) safety indices (FDA 2023), evokes inside a 7 T field peak electric intensity of 2.81 V/m (reference FIG. 9B). Such field strength can appreciably modulate neural activity. Fields as low as 0.3V/m produced by system 10 can modulate neuronal spiking. The clinically relevant transcranial electrical stimulation produces about 0.28V/m (95th percentile) in the human brain for the generally accepted maximum current of 2 mA.

System 10 was also used to test whether Lstim can modulate bioelectric signaling. Specifically, ultrasound delivery was focused from a distance of 52 mm onto a target that features intact nerves and receptors, the human thumb (reference FIG. 10B). Focused ultrasonic stimuli (reference FIGS. 10C and 10D) were delivered into the target every 8-12 s. Human subjects (n=18) were asked to report any nociceptive or tactile sensation. A nociceptive sensation results from activation of free nerve endings in the skin and thus constitutes a metric of neural activation.

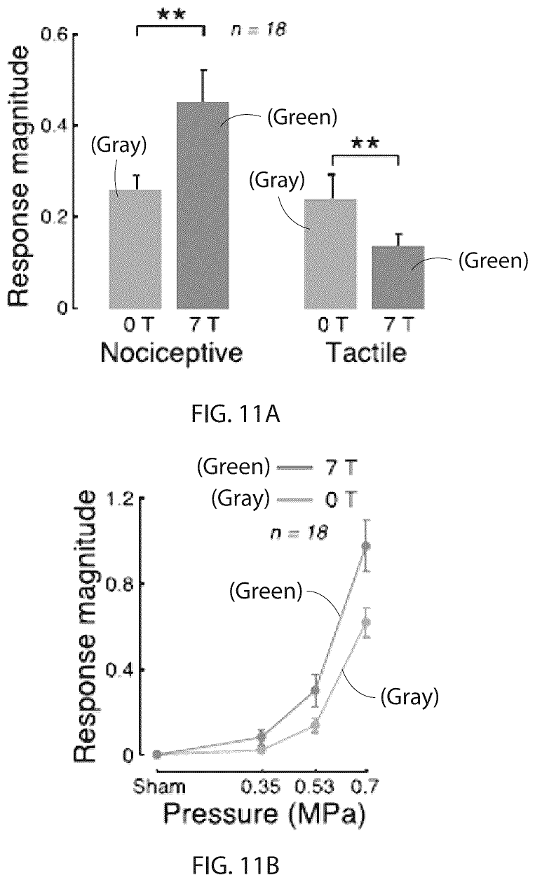

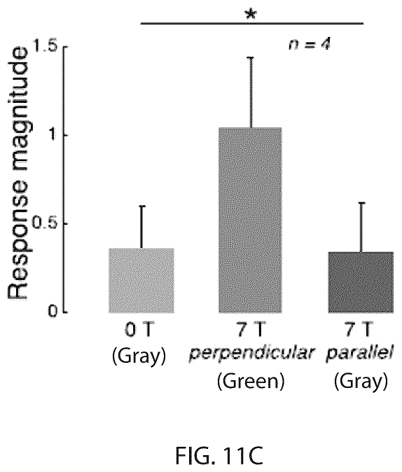

The magnetic field was found to substantially enhance the magnitude of nociceptive responses, as shown in FIG. 11A which displays means±SEM response magnitude for ultrasound alone, and ultrasound combined with 7 T magnetic field, separately for nociceptive responses as shown on the left and tactile responses as shown on the right. Data were pooled over all stimuli tested. The double stars indicate effects significant at p<0.01. Across all pressure levels and waveforms, Lstim increased the magnitude of nociceptive responses by 74%. In contrast to nociceptive responses, tactile responses were suppressed; there was a double dissociation of the effects with respect to magnetic field and the sensation kind (two-way ANOVA, magnetic field×sensation interaction, p<0.001; F(1, 644)=13.20). The effect was similar when subjects' responses were not scaled by their intensity (p<0.001; F(1, 644)=13.93). Pairwise post-hoc tests showed that the increase in the nociceptive responses (p=0.0059; t(17)=3.14, paired two-sided t-test) and the decrease in tactile responses (p=0.0033; t(17)=−3.41) were significant. These effects were also similar when the responses were not scaled by their intensity (p=0.0037; t(17)=3.36 and p=0.0029; t(17)=−3.47, respectively).

The nociceptive responses were analyzed, these responses reflecting an activation of nerves or nerve endings. FIG. 11B shows the dependence of all stimuli on the presence or absence of magnetic field, separately for each ultrasound pressure. Lstim-evoked nociceptive responses increase with ultrasound pressure. FIG. 11B shows mean±SEM magnitude of nociceptive responses as a function of ultrasound pressure at target and the presence (green) and absence (gray) of magnetic field. Data were pooled over all stimuli. The data of FIG. 11B confirms the findings of FIG. 11A, that the magnetic field amplifies the nociceptive responses. The effects of using a full, three-way ANOVA model were assessed, which factors magnetic field, ultrasound pressure, stimulus waveform, and all possible interactions (reference Table 1). The effect of magnetic field was significant also in this omnibus analysis (p<0.001, F(1, 408)=18.55).

Lstim produces focused electric fields at ultrasound targets according to

$$E = \frac{PB}{Z} .$$

In this equation, the effect increases with the ultrasound pressure P. Therefore, the higher the ultrasound pressure, the stronger the induced electric fields, and the stronger the nociceptive responses, in addition to any neuromodulatory effects of ultrasound alone. In line with this expectation, a significant interaction between the magnetic field and ultrasound pressure was found (reference FIG. 11B; p=0.0012, F(3, 408)=5.41).

The effects of all factors and interactions are summarized in Table 1. With respect to nerve activation, as assessed by the nociceptive responses, there was a significant interaction between magnetic field and the stimulus waveform (p=0.043, F(2, 408)=3.16). The contrast between Lstim and ultrasound only was higher when the ultrasound was pulsed by system 10. Specifically, averaged across all pressures, the response frequency ratio (7 T versus OT) for the continuous waveform was 1.61, compared to 1.85 and 3.85 for the pulsed 500 Hz and 10 KHz waveforms, respectively.

The reported effects are due to the induction of the localized electric field, as governed by the Lorentz electromotive force equation, and the effects depend on the orientation of the nerves with respect to the applied electric field. Specifically, electric fields can effectively stimulate nerves if their gradients point along nerves, as opposed to across the nerves. To test this, four subjects were asked to place their thumb on the aperture (1) in an orientation perpendicular to the magnetic field (the current default) and (2) in parallel with the magnetic field. FIG. 11C illustrates that Lstim activates nerves in an orientation-specific manner. Mean±SEM magnitude of nociceptive responses as a function of the orientation of the induced electric field with respect to the subjects' nerves is shown in FIG. 11C. The neuromodulatory effects are maximized when the nerves are aligned with the induced electric field (green). Data were pooled over all stimuli. The star indicates that the modulation by the magnetic field and its orientation was significant (p<0.05).

As shown in FIG. 11C, it was found that these conditions significantly modulated the responses (p=0.041, F(2, 33)=3.50). As expected, the effect was specific to the perpendicular geometry; there was no effect for the parallel geometry (p=0.88, t(3)=0.17, paired two-sided t-test).

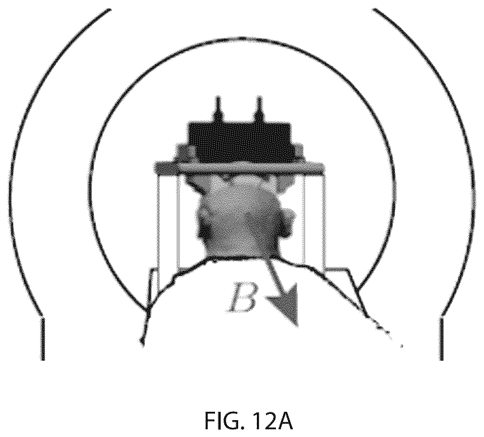

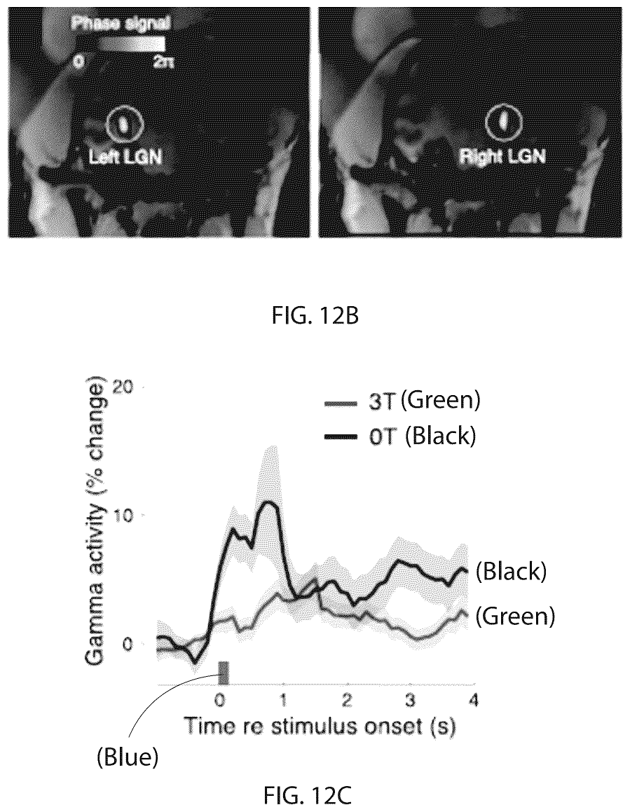

The effects of Lstim on deep brain regions of non-human primates were evaluated in study S3. FIG. 12A illustrates a system 10 comprising a 256-element, MRI-compatible phased array of ultrasound transducers that is inserted into a frame that is mounted into four titanium posts attached to the skull of two non-human primates (monkeys M1 and M2 described herein). Each monkey was positioned in a standard sphinx position. In this position, the magnetic field of the scanner (see arrow) points toward the reader. Since the ultrasound is delivered from the top, the induced Lstim field points along the monkey's left-right axis. Specifically targeted were the lateral geniculate nuclei (LGN), deep brain regions that pass visual information to visual cortex, reference FIG. 12B which provides MRI thermometry images that validate the LGN targeting. The images represent the selective targeting of the left and right LGN. A previous study showed that ultrasonic neuromodulation of the LGN increases gamma activity over visual cortex. In study S3, the same apparatus and recordings were used to assess the effects of Lstim. Referring to FIG. 12C, illustrated are mean±SEM high gamma activity recorded from the two posterior pins in response to 100 ms stimuli (480 KHz carrier frequency, 2 MPa amplitude) applied every 4 s to each LGN in a strictly alternating manner. The stimuli were either continuous or pulsed at 200 Hz pulse repetition frequency. Since there was no statistically significant difference, the data were pooled across these two conditions. Data are shown separately for the monkeys positioned inside the MRI (green) and 2 m outside of the MRI bore (black). The responses are aligned to the offset of each ultrasound stimulus (blue bar) and contain data of seven sessions recorded in the two monkeys, M1 and M2. As described hereabove, the stimulation was delivered by system 10 inside and outside of a static field of a 3 T MRI magnet every 4 s. The ultrasonic stimuli were 2 MPa in amplitude, 100 ms in duration, and were either continuous or pulsed at 200 Hz pulse repetition frequency. Replicating the previous findings, a robust increase in gamma activity over visual cortex following the ultrasonic stimulation was found (reference FIG. 12C; black). Crucially, the presence of the strong magnetic field had a profound influence on the induced gamma activity (reference FIG. 12C; green). In particular, the presence of the magnetic field dampened the gamma response, and led to a much more gradual increase following the stimulus onset. These effects were assessed using a two-way ANOVA, with factors magnetic field and stimulus type (continuous or pulsed). The gamma activity was measured in the time window immediately following the ultrasound offset (100 ms) up until the end of each trial (time 4 s). There was a significant effect of magnetic field (F(1, 981)=5.64, p=0.018). Stimulus type or the interaction of the two factors were non-significant (F(1, 981)=1.27, p=0.26 and F(1, 981)=1.06, p=0.30, respectively). In the time window considered, there was an average increase of gamma by 6.1% and 5.4% in monkeys M1 and M2, respectively, at OT, compared with 2.0% and 2.5%, respectively, at 3 T. No detrimental effects were observed during or after the stimulation. The monkeys showed normal behavior following the procedures.

DISCUSSION: Study S3 shows that a system 10 that is configured to produce a combination of magnetic and focused ultrasonic fields generates localized electric fields remotely and noninvasively. The resulting stimulation, Lstim, produces notable neuromodulatory effects, and study S3 demonstrated those effects in the peripheral nervous systems of humans and the central nervous system of non-human primates. The method can therefore be deployed for electrode-free modulations of neural and other processes that rely on electrical signaling.

System 10 is configured to provide Lstim with three major advantages as compared with traditional, electrode-based stimulation. The first, key advantage is that Lstim does not require the insertion of electrodes into a target to produce localized electric fields. The localization is achieved through the focusing of ultrasound. For high ultrasound frequencies, the stimulation focus delivered by system 10 can be as tight as a few dozens of micrometers (e.g., less than 100 μm, or less than 50 μm). Second, Lfield produces much sharper gradients and thus has a much higher potential for triggering bioeffects compared with electric fields generated with a pair of electrodes. Specifically, the Lorentz equation

$$E ( x , y , z ) = P ( x , y , z ) \frac{B}{Z}$$

shows that the spatial distribution of the electric intensity E(x, y, z) follows the distribution of the ultrasonic pressure wave P(x, y, z). Consequently, the propagating sinusoidal ultrasound pressure wave generates an E gradient with the peak positive and peak negative E values spaced by

$$\frac{λ}{2} .$$

At 258 kHz, this amounts to approximately 2.9 mm. In comparison, traditional electromagnetic fields, due to their much higher speed of propagation, have a

$$\frac{λ}{2}$$

that is approximately 231 m for the same frequency. Thus, due to the ultrasonic component, Lstim generates in the target electric field gradients that are five orders of magnitude stronger as compared to traditional electromagnetic fields. This difference is critical with respect to neurostimulation as neurostimulation effects are known to scale with an activating function

$$f ∝ \frac{dE}{dx} ,$$

where dE/dx is the gradient of the electric fields along the excitable structure. The Lfield gradients can be controlled by specific frequencies and waveforms of the ultrasonic pressure wave. And third, Lstim circumvents the barriers associated with biological membranes. Membranes are transparent to Lstim, due to the fact that membranes are transparent to magnetic and ultrasonic fields. Lstim's independence of biological membranes opens a new set of clinical applications that modulate intracellular processes remotely and more effectively than previously considered possible.

Notable effects were detected on peripheral nerves in humans at ultrasound pressures approximately three times lower than those allowable by current FDA 510(k) guidelines (0.7 MPa compared with 2.4 MPa or less than 190 W/cm² in soft tissues FDA 2023). At 2.4 MPa, still considered safe, the effects of Lstim would be more than three times stronger than those reported in study S3. Moreover, for the relatively low frequencies such as those used in study S3, ultrasound amplitudes higher than 2.4 MPa can be applied by system 10 in brief pulses without a risk of harmful heating. If even stronger effects are needed for certain clinical applications, the stimulation could be performed in stronger magnetic fields. Magnetic fields over 30 T are readily available.

Study S3 showed that Lstim as delivered by system 10 increases nociceptive responses and decreases tactile responses. The preferential engagement of nociceptive fibers is likely due to the sharp, mm-level gradients induced by Lstim. The gradients are likely to preferentially engage nerve structures with geometries on that order (i.e. nociceptive fibers), while the effects may average out over geometries that exceed the wavelength (i.e. tactile receptors and fibers). Moreover, this double dissociation suggests that Lfields delivered by system 10 modulated the electrical signals generated by skin receptors in response to ultrasound. This modulation can be applied to clinical applications for blockage of aberrant signaling, such as that involved in treatment of pain.

In study S3, system 10 was used to deliver pressures of 2.0 MPa into the deep brain targets of non-human primates to induce reliable changes in gamma activity recorded over visual cortex. It was found that the presence of 3 T magnetic field substantially dampened and slowed the gamma responses to ultrasound. The effect was present for at least 4 s, the duration of the interstimulus interval. Previous studies have used high-frequency continuous electrical waveforms for causing neural inhibition or conduction block. The relatively high carrier frequency of the ultrasound delivered by system 10 produces high-frequency waveforms, and thus the effect is consistent with those studies. It is possible that the relatively high pressure amplitude of 2.0 MPa produced by system 10 further amplified this effect. It is also likely that peripheral nerves and LGN neurons respond to this new mode of stimulation, which induces sharp gradients, in fundamentally distinct ways.

System 10 can deliver continuous stimuli, such as stimuli comprising a high-frequency carrier which suppresses neural activity. Alternatively or additionally, system 10 can deliver pulsed stimuli, such as pulsed stimuli that produce an “onset response” in which transient increases in neural activity follow the onset of a high-frequency stimulus. Both the human peripheral stimulation, as well as the monkey central nervous system stimulation, did not distinguish between the stimulus types. It is possible that the induction of a pronounced neural excitation with Lstim will require much higher pulse repetition frequencies than those considered here. In some embodiments, the pulse repetition frequency comprises a rate of at least 0.1 Hz, a rate of no more than 100 kHz, or both.

The noninvasive and targeted nature of Lstim as delivered by system 10 provides a new means for systematic modulation of specific neural targets in each patient, with the potential to realize the promise of precision medicine. System 10 can comprise arrays of transducers that focus ultrasound programmatically into neural targets that can be as small as a few dozens of micrometers when applied in soft tissues, and about 3 mm in diameter when applied through the human skull. Coupled with the microsecond-level temporal resolution of ultrasound, Lstim as provided by system 10 can activate multiple circuits in sequence or in concert. Together, the high spatiotemporal resolution of the system 10 energy delivery provides the means to modulate specific neural targets systematically. For instance, system 10 can be used to deliver Lstim to identify (e.g., and subsequently treat) the neural circuits that are involved in chronic pain of a particular patient. In addition, the ability to systematically manipulate specific brain circuits has the potential to transform current understanding of basic brain function.

For static magnetic fields, Lstim as delivered by system 10 produces stimulation of the same frequency as that of the applied ultrasound. The defined ultrasound frequency results in the delivery being immune to potential influence of external sources, unless they operate at the same frequency as that of the ultrasound.

In some embodiments, system 10 includes an MRI to deliver a magnetic field in delivery of Lstim. Alternatively, system 10 can comprise a set of one, two, or more coils to produce this magnetic field (e.g., a functional element 99 and/or 199 comprising one, two or more coils configured to deliver a magnetic field). Since no gradients and imaging are required, such systems could be produced much more affordably than a system 10 including an MRI.

Lstim as delivered by system 10 induces electric fields without the need for inserting electrodes into the target, thus preserving its integrity and sterility. This delivery configuration can provide clinical and other applications beyond neuromodulation, such as remote stimulation of tissue or cell cultures, food processing, or the catalysis of certain chemical reactions.

In summary, study S3 demonstrated that remote application of magnetic and ultrasonic fields by system 10 produces electrical stimulation that is both effective and safe. The high spatiotemporal resolution of the stimulation, enabled by system 10 ultrasound phased array, provides a new means to modulate biological processes flexibly and systematically. This systematic tool enables noninvasive modulations of spatially specific biological processes of a mammalian patient, including those in the nervous system.

### Study S4

As described herein, system 10 of the present inventive concepts can be used to treat pain, such as chronic pain, experienced by a patient, via direct intervention into deep brain circuits. Cingulotomy and deep brain stimulation targeting the anterior cingulate cortex have shown notable improvements in the unpleasantness of pain, but these interventions require brain surgeries. In study S4 described herein, system 10 is used to modulate this deep brain affective hub entirely noninvasively, using low-intensity transcranial-focused ultrasound as delivered by treatment device 100. Twenty patients with chronic pain received a 40-minute active or sham stimulation protocol and were monitored for one week in a randomized crossover trial. Sixty percent of patients experienced a clinically meaningful reduction of pain on day 1 and on day 7 following the active stimulation, while sham stimulation provided such benefits only to 15% and 20% of subjects, respectively. On average, active stimulation reduced pain by 60.0% immediately following the intervention and by 43.0% and 33.0% on days 1 and 7 following the intervention. The corresponding sham levels were 14.4%, 12.3%, and 6.6%. The stimulation was well tolerated, and no adverse events were detected. Side effects were generally mild and resolved within 24 hours. Together, the direct, ultrasonic stimulation of the anterior cingulate cortex as provided by system 10 offers rapid, clinically meaningful, and durable improvements in pain severity.

INTRODUCTION: An estimated 20% to 30% of people suffer from chronic pain, a type of pain that does not resolve following healing of the initial injury. Chronic pain is often recalcitrant, greatly diminishes the quality of life, and frequently results in psychiatric disorders, and, in some cases, suicide. Imaging and interventional studies provide compelling evidence for the involvement of a deep brain neural hub in the limbic system, the anterior cingulate cortex (ACC), in the unpleasant, aversive component of pain.

The ACC is heterogeneous in structure and function. Three ACC subregions: anterior midcingulate cortex (aMCC), pregenual ACC (pACC), and subgenual ACC (sACC), are associated with emotional regulation associated with chronic pain. Anatomically, the pACC is tightly linked to the prefrontal cortex, whereas sACC is connected with the amygdala. These regions show a functional dissociation with respect to emotional valence: the sACC and pACC are modulated by negative and positive emotions, respectively. The aMCC plays a role in the cognitive/evaluative aspect of chronic pain. The aMCC is activated by actual pain experience, as well as by pain related contextual cues. Furthermore, activity in this area is modulated by attention toward or away from painful stimuli.

Thus, the ACC serves a distinctive function of integrating the affective-cognitive parameters of pain perception. Indeed, preclinical models of neuropathic pain show a significant role of ACC in linking pain and depressive behaviors. Moreover, ACC hyperactivity accentuates the aversive component of chronic pain. The ACC also appears to integrate situational affective valence into the subjective experience of pain. For example, individuals who receive noxious stimulation while exposed to sad faces exhibit significantly greater activation of the ACC compared with individuals presented with happy faces. Reduced ACC activity following lesions can cause deficits in response selection to noxious stimuli, kinetic mutism, motor neglect and impaired motor ignition, and aberrant social behavior.

Surgical interventions into the ACC using cingulotomy are known to improve pain symptoms, which supports a causal role of this brain region in the processing of pain. Specifically, a systematic review that evaluated patients across 11 studies showed that this well-tolerated procedure provides pain relief in over 60% of cases. Deep brain stimulation (DBS) implants targeting the ACC provide an average of 35% to 48% reduction in pain intensity. Despite the effectiveness of these treatment options, both cingulotomy and DBS require surgeries, which greatly limit their scalability to benefit larger patient populations.

To address this issue, system 10 of the present inventive concepts can be used to modulate the ACC and associated circuits entirely noninvasively. System 10 can focus ultrasonic waves into deep brain targets through the intact skull and scalp. Critically, system 10 measures and compensates for the severe aberrations of ultrasound by the human head, thus delivering into each target controlled, deterministic ultrasound intensity. In study S4, system 10 was used to modulate the ACC in 23 patients with chronic pain. The primary aim of study S4 was to assess the effects on pain using a randomized crossover sham-controlled study design. In addition to the clinical outcomes, in a subset of patients, we validated target engagement using functional MRI (fMRI).

METHODS—Trial Design: Study S4 was a pilot double-blind, randomized, controlled crossover trial assessing the efficacy and safety of focused ultrasound (FUS) stimulation of the ACC using system 10 for participants (also referred to as “patients” or “subjects” herein) that are experiencing generalized chronic pain.

Subjects who met the study criteria completed baseline measures of chronic pain including the Brief Pain Inventory, Patient-Reported Outcomes Measurement Information System (PROMIS) pain intensity, PROMIS depression, and PROMIS anxiety metrics. Following baseline, subjects were randomly assigned to active or sham groups. Both groups started with an MRI session (approximately 1 hour) which was primarily used to register the treatment device 100 to the patient's brain anatomy. Secondarily, that session also measured fMRI activation in response to ultrasound stimulation (via an imaging device 800 comprising an MRI). Next, subjects participated in a treatment session outside of the MRI (40 minutes of stimulation; approximately 1 hour in total) using either completely active or sham stimulation. Subjects were monitored after treatment for 7 days and then crossed over to the opposite group if they continued to meet inclusion criteria. Subjects were required to have at least an average 24-hour visual analogue scale (VAS) pain score of 3 to crossover to the second treatment and delayed treatment after active or sham until this threshold was met. At the crossover, the subjects repeated the treatment procedure in the opposite group and were again monitored for 7 days post intervention.

METHODS—Participants: Study S4 recruited subjects with a primary diagnosis of chronic pain between the ages of 18 and 65. Pain had to be present for at least 3 months with moderate-to-severe levels of pain.

Exclusion criteria included any patient with a lifetime history of a serious suicide attempt; history of serious brain injury or other neurological disorder; brain stimulation in the past month (e.g., electroconvulsive therapy, transcranial magnetic stimulation [TMS], and/or vagal nerve stimulation); MRI intolerance or contraindication; and/or implanted device in the head or neck.

TABLE 3
Demographic information.

| | Real | Sham |
|---|---|---|
| Female (male) subjects | 12 (8) | 11 (9) |
| Age (SD) | 46.5 (10.36) | 46.7 (10.37) |
| Average VAS (SD) | 5.35 (1.39) | 5.21 (1.47) |
| PROMIS pain intensity 3a | 65.02 (4.75) | 63.60 (4.97) |
| PROMIS depression 8b | 59.19 (6.97) | 58.64 (8.48) |
| PROMIS anxiety 7a | 57.8 (9.86) | 58.39 (8.61) |

In Table 3, individual rows provide, separately for real (left) and sham (right) stimulation groups: the number of female (male) subjects, mean±SD age, mean±SD baseline visual analogue score of pain, and mean±SD baseline scores of PROMIS pain intensity, depression, and anxiety.

Table 3 summarizes the participant sample characteristics. The study population was 60% women and, on average, 46.6 years of age. The Brief Pain Inventory (BPI) 24-Hour Average Pain score ranged from 3 to 8 with an average of 5.35 for active and 5.21 for sham. Patient-Reported Outcomes Measurement Information System pain intensity score ranged from 54.2 to 74.4 with an average of 65.02 for active and 63.60 for sham. The study cohort corresponded to subjects with moderate-to-severe pain. Subjects had single or multiple sources of chronic pain that ranged broadly including fibromyalgia (10), myofascial pain syndrome (4), generalized pain syndrome (4), migraines (3), back pain (3), neuropathy (3), arthritis (3), chronic fatigue syndrome (2), complex pain syndrome, piriformis syndrome, atypical trigeminal neuralgia, cervical myelopathy, shoulder pain, foot pain, joint pain, endometriosis, scleroderma, dysautonomia, common variable immunodeficiency, temporomandibular joint dysfunction, Guillain-Barre syndrome, Crohn disease, and post-cancer pain.

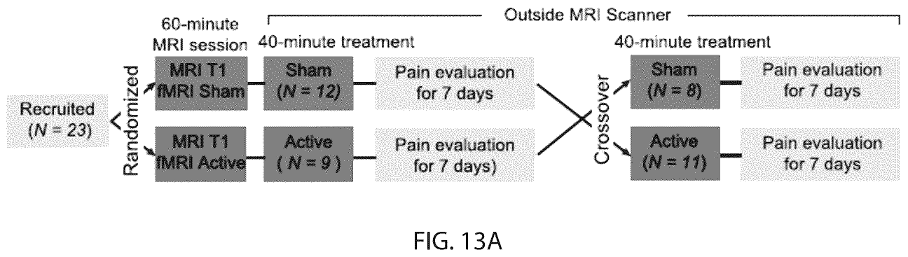

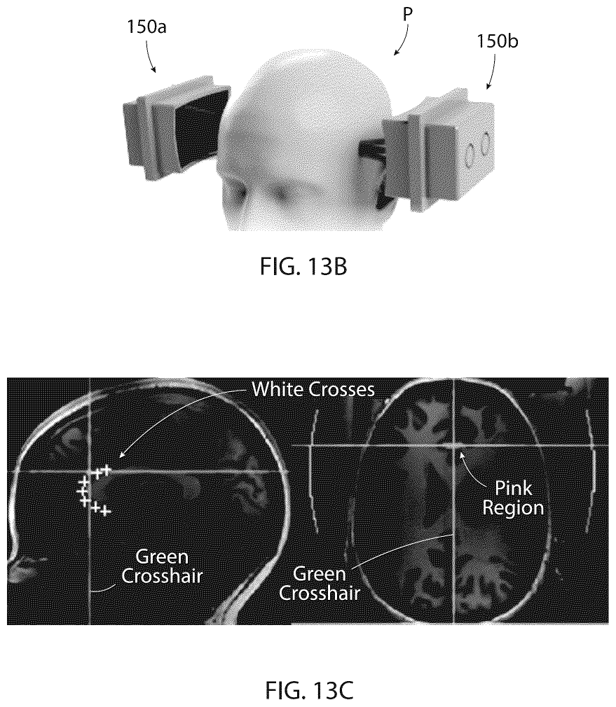

FIGS. 13A-C illustrate a flow chart, an anatomical schematic, and MRI images, respectively. In FIG. 13A, a flow chart of the trial design shows: randomization to MRI T1 and 10-minute fMRI measure of either active or sham stimulation followed by the first 40-minute ultrasound treatment session outside the MRI scanner, 7-day monitoring, washout, second treatment session outside the MRI scanner, and 7-day monitoring. There were 20 data points available for the active and sham conditions. In FIG. 13B, a schematic of how transcranial low-intensity focused ultrasound was delivered into the ACC target using a treatment device 100. In FIG. 13C, MRI images are shown that were used to validate the ACC targeting. Since the ACC is a large structure with respect to the ultrasound focus (2.4 mm by 3.6 mm by 20.4 mm), 8 ACC subregions were targeted, as indicated by the white crosses. The green crosshair exemplifies the targeting of one of the subregions. The pink regions outline the corresponding greater than 50% peak intensity focal volume of the ultrasound.

METHODS—Interventions: Study S4 evaluated FUS stimulation of the ACC over a single 1-hour session containing 40 minutes of active sonication. Ultrasound was focused on a target using a treatment device 100 comprising 2 phased array transducers placed over the left and right parietal bones. All treatments took place outside the MRI (reference FIG. 13A).

METHODS—Registration: Before treatment, subjects underwent a standard anatomical (T1-weighted) MRI for treatment guidance. These scans enable the co-registration of the position of treatment device 100 to subject-specific brain anatomy, such as via housing 110 and/or other components of system 10 as described herein. Treatments were performed outside the MRI. The patient's head was locked in a radiological mask (e.g., mask 700a, as described herein) and positioned as during the previous MRI scans, thus ensuring targeting reproducibility (reference FIG. 13B).

METHODS—Targeting: Following registration, 8 targets within the ACC were selected: 2 targets within the subgenual ACC (Brodmann Area 2565) and 6 targets from within the pACC to aMCC (Brodmann Areas s24, p24, a24, 33). The arrays produce a 26 dB intensity field with lateral, elevational, and axial dimensions of 2.4 mm by 3.6 mm by 20.4 mm (y, z, and x dimensions of the Montreal Neurological Institute [MNI] coordinate system) (reference FIG. 13C). Each of the 8 targets was centered on the subject's midline in the x-dimension and white matter tracks from both hemispheres in the y-z dimension. Each target was separated from the adjacent target by 4 mm in the sagittal plane (y-z dimension) to provide a continuum but avoid an overlap of the stimulated subregions (reference FIGS. 13A-C). In this plane, the target was also placed at least 4 mm from the outer edge of the corpus callosum to minimize direct stimulation of this highly connected area. The shape of the focus allowed for bilateral stimulation of the ACC. Average target location and distribution are shown in FIG. 13C.

METHODS—Treatment: The treatment session, performed outside the scanner, consisted of 2 stimulation blocks. Block A contained sixteen 30-second stimulations to test for immediate symptom reduction. Symptoms were assessed verbally after each sonication with subjects reporting any positive or negative changes to their pain. The targets were ordered randomly without replacement such that each target was stimulated twice. Verbal reports typically took 15 to 60 seconds between each sonication. The 4 targets were selected that yielded the strongest reduction in the pain symptoms. Block B delivered to these 4 targets twelve 3-minute sonications, randomly interleaved. The individual stimulations were again spaced by 15 to 60 seconds as the operator selected the next target and the subject reported any positive or negative changes from the previous stimulation. Sham stimulation used the same protocol but only provided auditory masking to the subjects; not a voltage to the transducers of treatment device 100.

METHODS—Stimulation Parameters: Ultrasound was delivered to each target with amplitude of 1 MPa (estimated using the relative through-transmit skull correction, spatial peak pulse average intensity (I_{SPPA}) 31.0 W/cm², mechanical index (MI)=1.2, thermal index (TI)=to 0.64), 30 milliseconds burst duration containing pulses of 5 milliseconds on and 5 milliseconds off (duty cycle=50%), separated by 0.7-second burst interval (pulse repetition frequency (PRF)=1.42 Hz, spatial peak temporal average intensity (I_{SPTA})=0.66 W/cm²). Thermal index at target was calculated using W/W_{deg}, with W=310,000 W/m² and W_{deg}=ΔTρC/(2αf)=480,000 W/m², where ΔT is 1 degree of temperature change, ρ is density of brain tissue, 1030 kg/m³, C is specific heat of brain tissue, 3630 J/(kg K), a is the absorption of brain tissue, 6 MHz⁻¹ m⁻¹, and f is frequency, 0.65 MHz. Potential skull heating was assessed with both simulations and measurements inside ex vivo human skulls in previous work, finding a maximum 0.047° C. temperature increase for a 30-millisecond pulse.

METHODS—Relative Through-Transmit Skull Correction: System 10 provides the ability to directly measure and compensate for the attenuation of ultrasound by the patient's head and hair, as described herein. In brief, in the method used in study S4, the transducers (energy delivery elements 155) of treatment device 100 sequentially emitted a 10-cycle, low-intensity, 650 kHz pulse from each individual element while recording responses from all the other, non-transmitting elements. This through-transmit procedure enables a direct measurement of the ultrasound attenuation and phase shift by the skull and other obstacles in the transmission path, including the hair and the acoustic coupling of system 10. The through-transmit method is relativistic, performed in comparison to reference measurements taken in water for the same fixed geometry of the transducers. The relative differences in the received ultrasound waveforms between the two conditions enable the computation of the attenuation and phase shift the ultrasound experiences from each element to the target. The values are then used to scale the amplitude of each beam by the inverse of the estimated attenuation and to delay the emission time by the estimated phase shift. This approach restores the amplitude and field at the target.

METHODS—Sham Stimulation: Sham stimulation used auditory masking. Both the sham and active groups of subjects wore headphones during the intervention. The headphones delivered white noise combined with the sound of prerecorded ultrasound transmission pulses. These auditory stimuli were time-locked to the ultrasound stimuli during active stimulation to mask any sound associated with the ultrasound delivery. No ultrasound was delivered with the auditory masking during the sham stimulation.

METHODS—MRI acquisition: MRI acquisition was conducted with a Siemens VIDA 3 T system. Data collected included fMRI BOLD, high-resolution anatomical magnetization-prepared rapid gradient-echo (MPRAGE), and two opposite phase-encoded spin-echo field maps. Data acquisition included the following sequences: fMRI BOLD (T2*-weighted): interleaved series, posterior-anterior (P-A) phase encoding, repetition time (TR) 2.0 seconds, time to echo (TE) 33 milliseconds, flip angle (FA) 80°, field of view (FOV) 207 mm, 52 slices, slice thickness 2.4 mm, bandwidth 2004 Hz/pixel, echo spacing 0.62 milliseconds, and 300 volumes per 10 minutes; MPRAGE anatomical: ascending series, A-P phase encoding, TR 2.4 seconds, TE 2.26 milliseconds, FA 8°, 192 slices, slice thickness 1.3 mm, bandwidth 200 Hz/pixel, and echo spacing 6.84 milliseconds; spin-echo field maps: interleaved series, A-P and P-A phase encoding, TR 9.5 seconds, TE 66 milliseconds, FOV 207 mm, 52 slices, slice thickness 2.4 mm, bandwidth 1162 Hz/pixel, echo spacing 0.96, and echo-planar imaging (EPI) factor 86.

The MRI visit served primarily to acquire an anatomical MRI of the patient for device-to-patient registration. The secondary objective of the session was to explore fMRI BOLD activity during simultaneous ultrasound stimulation of the ACC.

METHODS—MRI Processing: A minimal fMRI processing pipeline was implemented to enable individualized analyses in patient-specific, native MRI space. Functional MRI processing was performed using Analysis of Functional NeuroImages (AFNI 24.0.04), ANIMA (3.0), and Statistical Parametric Mapping 12 (SPM12 r7219) software packages. Processing was completed in 5 steps: reduction in BOLD outlier voxel signals (despiking) (AFNI), EPI BOLD distortion correction utilizing opposing phase-encoded spin echo field maps (AN-IMA), BOLD time-series spatial realignment to 10th volume (SPM12), BOLD time-series slice time correction (SPM12), and spatial smoothing of time-series BOLD with 8-mm Gaussian Kernel (SPM12).

Special preprocessing consideration was given due to potential artifacts and MRI distortions that may be caused by the presence of ultrasonic transducers and water-based hydrogel coupling (PVA Hydrogel, UltrasoundCoupling.com) inside the MRI. These considerations led to the construction of a multipackage, study-specific processing pipeline. In particular, AFNI despiking was found to most accurately and consistently identify and remove gel coupling outlier signals from the EPI BOLD images. After despiking, all data were visually quality checked for removal of gel coupling signal before further preprocessing.

METHODS—Functional MRI Analysis: Functional MRI BOLD activation individual analyses were conducted in SPM12 using a whole-brain general linear model (GLM). The design contrasted 5 interleaved 1-minute epochs of stimulation with five 1-minute epochs of rest throughout a 10-minute BOLD scan time. Stimulation blocks followed parameters used in the treatment session outside the scanner but used a 1-minute total sonication block duration. The data, for both active and sham stimulation, were analyzed using 2 directional t tests contrasting stimulation and non-stimulation blocks. BOLD significance was determined with the inclusion of uncorrected signal values of P<0.001 and subsequent cluster analysis of P<0.05. Head movement was minimized by system 10 itself, which used an included stereotactic radiotherapy thermoplastic mask as described herein (e.g., Aquaplast RT Open Eye and Mouth Slimline U-Frame; QFix). Steps were taken to maximize sensitivity in the fMRI data processing. First, individuals were analyzed in their native-subject space to decrease spatial distortions due to normalization to standard MNI space. Second, movement parameters were not included in first-level subject-space GLMs. This is because, in block-design fMRI experiments, the inclusion of movement parameters in subject-level GLM analyses decreases the sensitivity of the GLM to detect BOLD modulation.

METHODS—Functional MRI Whole-Brain Group Analysis: Whole-brain, voxel-wise analysis was performed to define brain activity changes associated with ultrasonic neuromodulation. To enable group-level activation analysis, in addition to the steps for first-level individual analyses, the following fMRI processing steps were performed following the slice-time correction: co-registration of high-resolution T1 to mean of realigned time series (SPM12), normalization of co-registered T1 to MNI space using Advanced Normalization Tools (ANTS), normalization of timeseries BOLD by application of T1 normalization deformation fields (ANTS), and spatial smoothing of time-series BOLD with 8-mm Gaussian Kernel (SPM12). Two whole brain directional t tests were performed comparing rest blocks to sonication blocks (Off>On and On>Off t tests) to reveal decreases and increases in brain activation corresponding to active sonication.

METHODS—Clinical Assessments: The primary treatment efficacy outcome was the difference between active and sham FUS using the BPI average 24-hour pain intensity scores pre-intervention and post-intervention. Brief Pain Inventory scores were completed daily for 7 days following the intervention. Secondary outcome measures were the PROMIS pain intensity, PROMIS depression, and PROMIS anxiety. Verbally reported average VAS pain score was also recorded throughout the treatment session to assess the immediate effects of stimulation provided by system 10.

The safety of the focused ultrasound (FUS) delivered by system 10 in study S4 was assessed using a collection of spontaneously reported adverse events and General Assessment of Side Effects that were recorded at baseline and 24 hours after both the active and sham treatment sessions.

METHODS—Statistical Analysis: The primary outcome of BPI average 24-hour pain intensity over the 7-day monitoring period between the sham and active group was compared using a repeated measures analysis of variance, with Greenhouse-Geisser-adjusted P value for multiple comparisons. Individual post treatment days were compared between groups using a 2-sample t test with Bonferroni-Holm correction for multiple comparisons. Secondary outcomes of difference between groups in immediate pain reduction, PROMIS pain intensity, PROMIS depression, and PROMIS anxiety were all compared using the Wilcoxon signed-rank test to account for a nonnormal distribution of scores.

METHODS—Randomization: Randomization was conducted by a volunteer not involved with the data collection who prepared envelopes before the trial with notes designating “active” or “sham” sealed inside. Before the start of each treatment, an envelope containing the random designation was given to the person operating system 10 and shared with no one else who interacted with the patient. Patients were not informed of their group allocation. Clinicians, study coordinators, and researchers applying the device to the patient during the MRI and treatment sessions were all blinded.

RESULTS—Study Design: This study involved a randomized sham-controlled crossover design, in which patients were randomly assigned into active or sham treatment and crossed into the opposite arm 1 week later (FIG. 13A). Twenty-three patients with chronic pain were recruited for this study (refence Table 3). Two patients who started in the sham arm both completed the sham arm, then declined efforts to be contacted and did not crossover to the second arm. Neither subject experienced side effects related to the sham treatment. Their results and safety data are included in the analyses.

There was no dropout for patients who started in the active arm, although 1 patient who went into pain remission did not complete the crossover into sham. Overall, 20 active and 20 sham data points were available for analysis of clinical effects.

RESULTS—Targeting: The ultrasound delivery performed using system 10 in the treatment session was performed outside the MRI using the registered T1 MRI scan of the patient's brain and treatment device 100 transducers, as described herein.

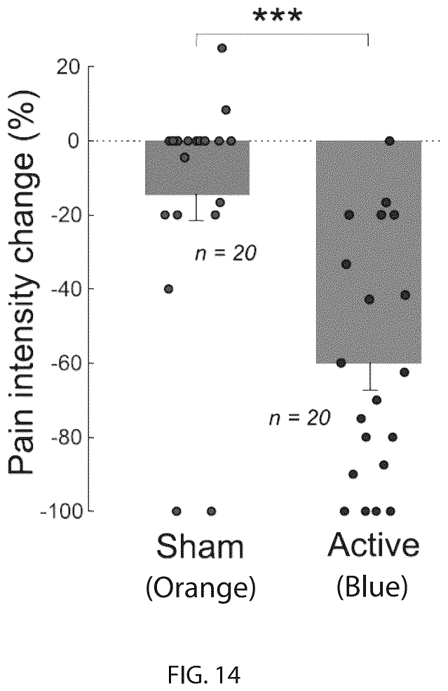

FIG. 14 is a bar chart illustrating pain intensity change percentage for sham and active stimulation of the ACC. As shown in FIG. 14, rapid changes in pain intensity followed ultrasonic modulation of the ACC. Shown are mean±SEM change in VAS scores in response to sham (orange) and active (blue) stimulation of the ACC, relative to the VAS scores before intervention. The individual data points are provided as shaded circles. In FIG. 14, *** represents P=0.00013, Wilcoxon signed-rank test.

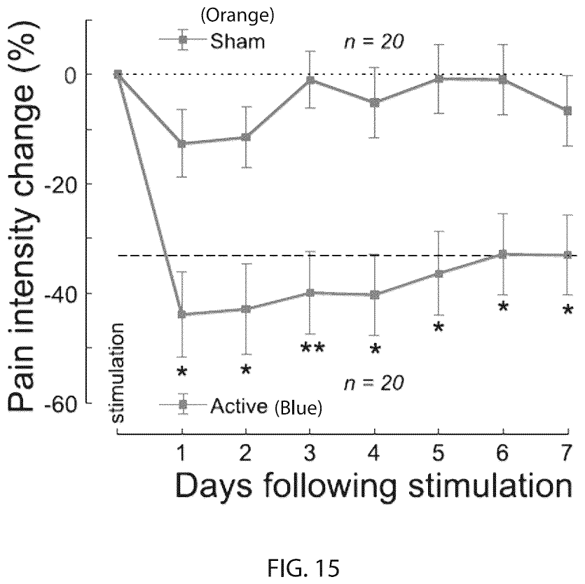

FIG. 15 is a graph of pain intensity change over time for sham and active stimulation of the ACC. Durable changes in pain intensity following ultrasonic modulation of the ACC using system 10 are shown, including mean±SEM change in VAS scores relative to baseline VAS scores taken before each intervention. The effects were measured for up to 7 days (abscissa). Data are provided separately for the active (blue) and sham (orange) stimulation. The dashed line represents a pain reduction level that is considered clinically meaningful. The stars denote significant differences between the active and sham effects (*P<0.05, **P<0.01; Bonferroni-Holm-corrected for multiple comparisons).

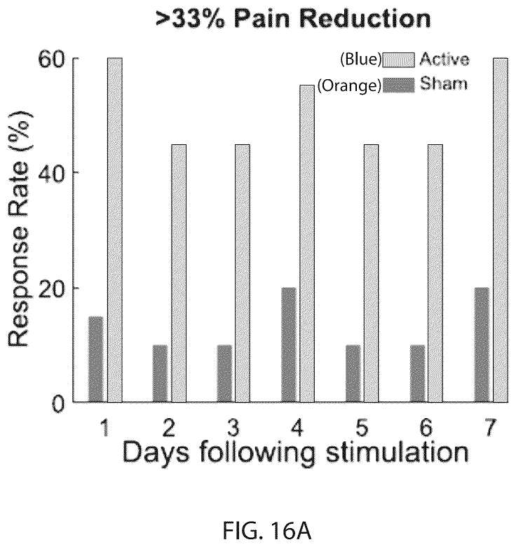

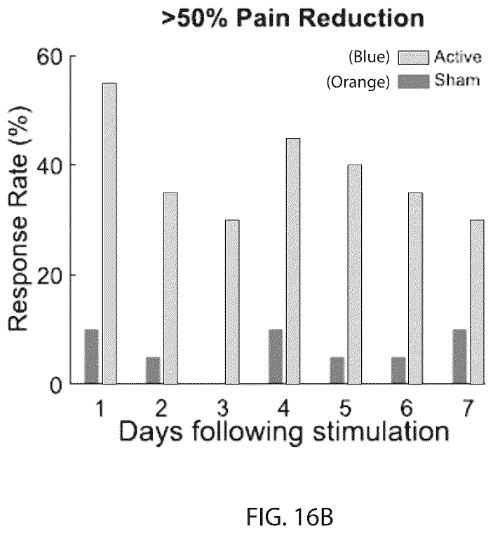

FIGS. 16A-B are two bar charts representing response rates comparing active and sham stimulations. In a similar format as FIG. 15, FIG. 16 shows the proportion of subjects who attained greater than 33% (FIG. 16A) and greater than 50% (FIG. 16B) reduction in pain intensity.

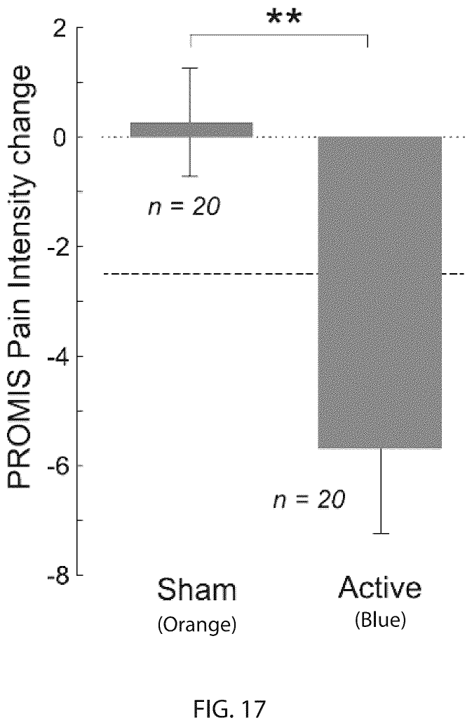

FIG. 17 is a bar chart representing PROMIS Pain Intensity change comparing sham and active stimulations, including the change in PROMIS pain intensity scores at day 7 following ultrasonic modulation of the ACC using system 10. Shown are mean±SEM changes in the PROMIS scores at day 7 following the stimulation, relative to PROMIS scores before the stimulation. The dashed line provides the level of clinically meaningful reduction in pain. As shown in FIG. 17, **P=0.0014, Wilcoxon signed-rank test.

RESULTS—Modulation of Pain: The ultrasound was delivered into the individual ACC subregions over a period of 40 minutes. Standard VAS scores and PROMIS scores were measured for up to 7 days following the single intervention. Immediately following active stimulation, patients reported reduced VAS pain scores of 60.0±33.1% (mean±SD; reference FIG. 14). This corresponds to an absolute VAS change of −2.7±1.4. By contrast, the sham stimulation, which only delivered the auditory masking sounds (reference Methods) and no ultrasound, resulted in a 14.39±32.15% reduction. The difference was highly significant (P=0.00013, z=3.83, Wilcoxon signed-rank test). Following the active treatment, 75% (15 of 20) of patients reported a clinically meaningful (33%) reduction in pain, with 60% (12 of 20) of patients reporting a reduction greater than 50%. By contrast, following the sham treatment, 15% of subjects experienced a clinically meaningful reduction in pain and 10% (2 of 20) experienced a reduction greater than 50%. Together, these data suggest that the ultrasonic ACC treatments as provided by system 10 resulted in a substantial reduction of pain levels following a single-session intervention.

The durability of pain relief following the single-session treatments was evaluated (FIG. 15). Pain reduction following active stimulation, but not sham stimulation, was particularly pronounced in the days following the intervention and remained statistically and clinically significant through the 7-day follow-up period (FIG. 15). A repeated measures analysis of variance confirmed a significant effect of the active treatment arm over the sham treatment arm (group effect: active or sham; F₇=3.21, P=0.0086, Greenhouse-Geisser adjusted). Following active treatment 60% (12 of 20) of patients reported a clinically meaningful reduction in pain at 24 hours and 7 days post, with 55% and 30% of patients reporting a reduction greater than 50% at these timepoints, respectively. By contrast, following the sham treatment, 15% (3 of 20) and 20% experienced a clinically meaningful reduction in pain and 10% experienced a reduction greater than 50% at 24 hours and 7 days post (reference FIGS. 16A-B)

The beneficial effects of ACC neuromodulation provided by system 10 in study S4 were also observed in the PROMIS pain intensity scores (reference FIG. 17). Active stimulation resulted in a mean±SD decrease of 5.68±7.2 points in the PROMIS pain intensity scores, and this effect was significant relative to the sham stimulation (P=0.0014, z=3.20, Wilcoxon signed-rank test). In the active FUS group, 55% (11 of 20) of subjects' pain scores improved by at least the clinically significant change of 2.5, compared with 17% (3 of 19). The PROMIS depression score decreased by 2.27±3.75 for active and 0.23±6.16 for sham (P=0.14, z=1.48, Wilcoxon signed-rank test). The PROMIS anxiety score decreased by 2.87±6.21 for active, and 0.65±5.36 for sham stimulation (P=0.20, z=1.29, Wilcoxon signed-rank test)

RESULTS—Safety: The stimulation was well tolerated with no adverse events detected. There were no significant differences between active and sham stimulation for any of the measured symptoms (reference Table 4 herebelow). All side effects related to treatment were resolved by the end of this study. There was no significant difference in the dropout rates between the active and sham conditions (P=0.49, Fisher exact test). No significant worsening of pain, measured with the Brief Pain Inventory, was observed in either the sham or active treatment groups.

TABLE 4
The stimulation was well tolerated by patients.

| | Real (N = 20) | | | | | Sham (N = 20) | | | | |
|---|---|---|---|---|---|---|---|---|---|---|
| | No. of adverse events | | | | | | | | | |
| | 0 | | | | | 0 | | | | |
| | Study dropouts | | | | | | | | | |
| | 0 | | | | | 2 | | | | |
| | [symbol] ASE rating | | | | | | | | | |
| | Not | | | | Related to | Not | | | | Related to |
| | present | Mild | Moderate | Severe | treatment (%) | present | Mild | Moderate | Severe | treatment (%) |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |

[symbol]

indicates data missing or illegible when filed

Following the stimulation of study S4, the patients were asked to complete a standard clinical questionnaire that assessed potential side effects. In Table 4, the data are shown separately for the active (left column) and sham (right column) stimulation

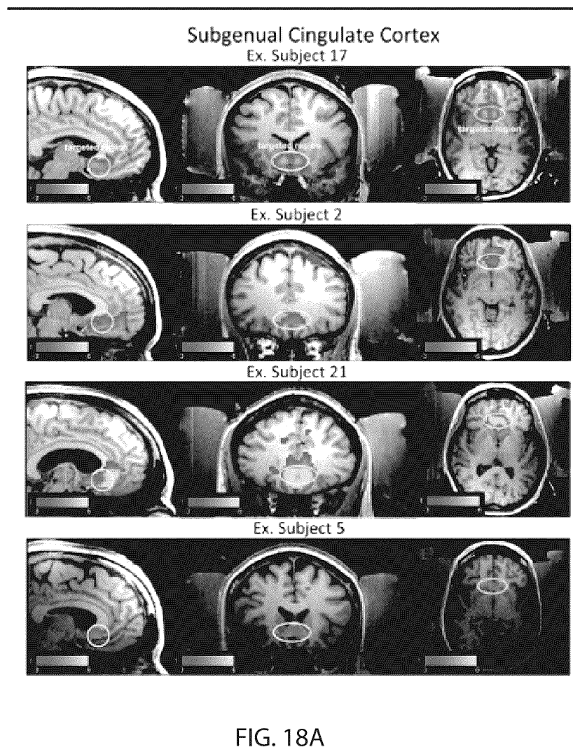

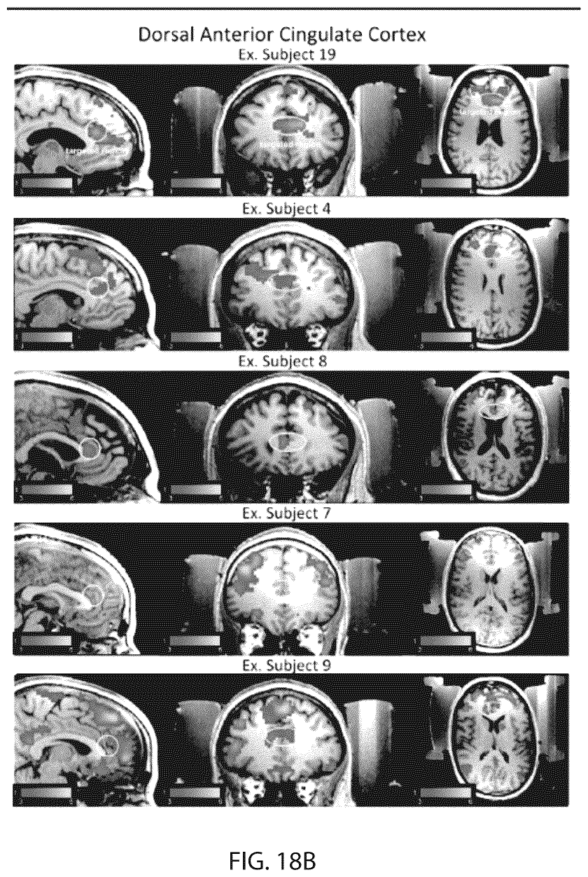

FIGS. 18A-B represent MRI images of the subgenual cingulate cortex (subjects 17, 2, 21, and 5), and dorsal anterior cingulate cortex (subjects 19, 4, 8, 7, and 9), respectively. Data for all subjects that received active stimulation, for subgenual ACC: subject 17 (cluster-level P<0.0001; false discovery rate corrected, k_{E}=369 voxels), subject 2 (cluster-level P<0001; false discover rate corrected, k_{E}=414), subject 21 (cluster-level P<0.0001; false discovery rate corrected, k_{E}=5791 voxels), subject 5 (signal inclusion P<0.005; cluster-level P<0.016; false discovery rate corrected, k_{E}=183 voxels). For aMCC: subject 19 (cluster-level P<0.0001; false discovery rate corrected, k_{E}=1344 voxels), subject 4 (cluster-level P<0.0001; false discovery rate corrected, k_{E}=1992 voxels), subject 8 (cluster-level P=0.034; uncorrected, k_{E}=47 voxels). Subjects 7 and 9 did not show significant activation at the target region. Group analysis was insignificant for both targets.

RESULTS—Functional MRI Target Engagement: Before the treatment session outside the MRI, sham and real stimulation were delivered inside the MRI to validate the ultrasound focus, using a target engagement procedure. Specification validation was performed to confirm that system 10 can effectively target both the ventral (subgenual) and dorsal parts of the ACC.

Significant effects were observed at target in 3 of 4 patients in whom the engagement of the subgenual ACC was tested, and 3 of 5 patients in whom the engagement of the dorsal ACC was tested (reference FIGS. 18A-B). Within the patients who showed significant effects, all subgenual ACC patients showed deactivation at target, while 2 aMCC patients showed target activation and 1 aMCC patient showed target deactivation. Second-level group analyses showed no significant clusters for the 4 patients who received subgenual ACC, the 5 patients who received aMCC, or the 10 patients who received sham.

DISCUSSION: Study S4 provides support for an exciting new treatment approach for chronic pain, using noninvasive FUS to stimulate the deep brain targets known to reflect pain experience. The results demonstrate that system 10 of the present inventive concepts can modulate deep brain structures in humans precisely, noninvasively, and in a controlled manner. In this approach, a treatment device 100 comprising a phased array device delivers ultrasound into specified deep brain targets, while measuring and compensating for the severe aberrations of ultrasound by the head, as described herein. Using this approach, low-intensity ultrasound was administered to the ACC of patients with chronic pain. A randomized crossover sham-controlled evaluation revealed that the intervention provides a rapid, clinically meaningful, and durable reduction in chronic pain.

Rapid improvements in chronic pain can be obtained using medication treatments or surgical interventions, including cingulotomy and DBS. Nonetheless, medication treatments require frequent re-administration, often carry significant side effects, and can be addictive. On the other hand, the surgical options carry significant risks, including brain hemorrhage and infection. The ultrasound waves delivered by system 10 provide an alternative, drug-free, and incisionless treatment option for chronic pain.

Ultrasonic waves provided by system 10 combine a unique triad of properties: noninvasiveness, depth penetration, and sharp focus. Since sound waves have a much lower speed of propagation than electromagnetic waves, sound waves have a relatively small wavelength. Thanks to diffraction, the short wavelength enables relatively sharp focus at depth. Nonetheless, the technology has been impeded by formidable barriers: the skull and hair, which attenuate and distort ultrasonic waves severely and unpredictably. The approach presented in study S4 and provided by system 10 directly measures and compensates for these barriers in each individual, thus delivering into specified targets a controlled, deterministic amount of ultrasound intensity. This way, low-intensity ultrasound can provide targeted noninvasive neuromodulation in an effective and safe manner.

At the mechanistic level, delivery of ultrasound energy via system 10 mechanically activates ion channels and directly elicits action potentials. When ultrasound is delivered into neural tissues for dozens of seconds or longer, it also induces neuroplastic effects. These effects are, at least in part, due to activation of glial cells. In addition, these effects are a function of the ultrasound parameters provided by system 10. Careful modeling work has suggested that low-energy insonations produce aggregate neuroinhibitory effects, whereas higher energy insonations tend to produce neuroexcitatory effects. Ultrasound-induced neuroplastic effects provide a unique opportunity for noninvasive reset of the malfunctioning circuits. A durable reset based on induced neuroplastic effects is the primary hypothesized mechanism underlying the sustained effects reported in study S4.

In study S4, the bi-directional polarity of modulated fMRI BOLD signals was observed, despite the use of low-energy insonations. This bi-directionality between activation and deactivation at the target may be due to biological differences between the subgenual ACC and the aMCC. For the statistically significant patients, ultrasound to the subgenual ACC resulted in deactivation at the target. Similarly, this deactivation was largely restricted to the ultrasound target region. By contrast, 2 of 3 patients with significant modulation at the aMCC showed activation in the target region. Moreover, this aMCC modulation was consistently paired with changes in activity superior to the target in the dorsal medial prefrontal cortex, regardless of activation or deactivation at the target. Taken together, the study S4 data suggest that neuromodulatory effects of ultrasound may be not only parameter dependent, but also brain-region-dependent. Indeed, the subgenual ACC and aMCC have unique cellular architectures, with the aMCC containing both larger cell bodies and higher glia-to-neuron ratios. As noted, ultrasonic-induced neuroplastic effects are in part, due to activation of glial cells

Three lines of evidence support the notion that the ACC modulation effects of study S4 were due to the stimulation and not due to a generic artifact. First, there was a substantial and significant contrast in the reduction of the pain intensity levels between active and sham stimulation. This difference is even though the sham stimulation controlled for placebo using an active auditory protocol. Second, the active stimulation elicited localized effects and did not elicit a consistent activation of the auditory or somatosensory cortex (reference FIGS. 18A-B). Finally, the lack of MRI BOLD activation at target in some of the patients provides negative control data, implying the observed modulation in other patients was not due to a pervasive generic artifact.

System 10 of study S4 includes an imaging device 800 comprising an MRI that was used to register treatment device 100. In some embodiments, system 10 is configured to register treatment device 100 without the use of MRI.

In some embodiments, system 10 is configured to deliver both transcranial ultrasound stimulation, and transcranial magnetic stimulation, as described herein. The effects of both modalities may be complementary, and their combined application may provide stronger effects than either approach alone.

In summary, study S4 demonstrated a successful, noninvasive targeted approach to modulate the deep brain circuits involved in chronic pain. Using this approach, system 10 can provide effective, rapid, and durable reduction in chronic pain. The procedure is incisionless, medication-free, and can be applied to patients within minutes. This approach could therefore be administered to a large spectrum of patients, potentially contributing to the effort of reducing the administration of opioid medications or drugs that cause systemic side effect.

TABLE S1
Data of all subjects included in the analysis

| | | | | | | | | | [symbol] | | [symbol] | | | | | | | | [symbol] | | [symbol] | | | | | | | |
|---|---|---|---|---|---|---|---|---|---|---|---|---|---|---|---|---|---|---|---|---|---|---|---|---|---|---|---|---|
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |

[symbol]

indicates data missing or illegible when filed

System 10 and its components can be configured and used as described in reference to study S4 and otherwise herein, such as to treat a patient as described in reference to study S4 and otherwise herein, such as to achieve the benefits of treatment achieved using system 10 as described in reference to study S4 and otherwise herein.

A patient selected for treatment can comprise a patient that has an average 24-hour visual analogue scale pain score of at least 1, such as at least 3. The patient selected can have experienced at least 3 months of a moderate level of pain. The target location (e.g., the one or more anatomical locations into which energy is delivered) can comprise the anterior cingulate cortex (ACC). The target location can comprise at least two, or at least four target locations within the ACC. At least four target locations can comprise four locations selected from the group consisting of: subgenual ACC (Brodmann Area 2565) and 6 target locations from within the pACC to aMCC (Brodmann Areas s24, p24, a24, 33). At least four target locations can comprise eight target locations within the ACC. Each target location can be separated from a neighboring target location by at least 2 mm, by no more than 6 mm, or both. (e.g., to provide a continuum of tissue stimulation but avoid an overlap of the stimulated subregions). Each target location can be separated from a neighboring target location by approximately 4 mm. The energy delivery can comprise a delivery of energy with a peak intensity of no more than 300 W/cm². The energy delivery can comprise a delivery of energy with a peak intensity of no more than 225 W/cm², and/or no more than 190 W/cm². The energy delivery can comprise a delivery of energy with a duration of no more than 3 hours, no more than 2 hours, and/or no more than 1 hour. The energy delivery can comprise a delivery of energy with a duration of at least 30 minutes, of no more than 120 minutes, or both. The energy delivery can comprise a delivery of energy for approximately 40 minutes. The energy delivery can comprise a delivery of energy with field dimensions less than 5 mm by 5 mm by 40 mm. The energy delivery can comprise a delivery of energy with field dimensions of approximately 2.4 mm by 3.6 mm by 20.4 mm. The delivery of energy can comprise multiple test energy deliveries configured to test for symptom reduction (e.g., to determine that a proper target is receiving the energy). Each test energy delivery can comprise a delivery of energy for no more than 60 seconds. The energy delivery can comprise multiple energy deliveries configured to provide a therapeutic benefit to the medical condition. The multiple energy deliveries can comprise at least four energy deliveries of at least one minute in duration. The multiple energy deliveries can comprise approximately 12 deliveries of energy, each of three minutes in duration. The energy delivery can comprise a delivery of energy with an amplitude of at least 0.5 MPa. The energy delivery can comprise a delivery of energy with an amplitude of approximately 1 MPa. The energy delivery can comprise a delivery of energy comprising bursts of at least 10 msec in duration that can be delivered at less than 90% duty cycle. The burst can be separated by a burst interval of no more than 20 seconds. The energy delivery can comprise a delivery of energy comprising bursts of approximately: 30 msec in duration; 50% duty cycle; and 0.7 second burst interval. The treatment provided by system 10 can achieve an efficacy comprising one or more of: at least a 30% reduction in pain immediately following energy delivery; at least a 21.5% reduction in pain at 1 day after energy delivery; and/or at least a 16.5% reduction in pain 7 days following energy delivery. The treatment provided by system 10 can achieve a reduction in absolute VAS pain score of at least one. The treatment provided by system 10 can achieve a reduction in absolute VAS pain score of 2.7±1.4. The treatment provided by system 10 can have at least a 50% expectation of achieving a 33% reduction in pain immediately following the energy delivery. The treatment provided by system 10 can have an approximately 75% expectation of achieving a 33% reduction in pain immediately following the energy delivery. The treatment provided by system 10 can have at least a 30% expectation of achieving a 33% reduction in pain 24 hours after the energy delivery. The treatment provided by system 10 can have approximately a 60% expectation of achieving a 33% reduction in pain 24 hours after the energy delivery. The treatment provided by system 10 can achieve at least a 10% reduction in PROMIS pain intensity score. The treatment provided by system 10 can achieve a reduction in the PROMIS pain intensity score of mean±SD reduction of 5.68±7.2 points. The treatment provided by system 10 can achieve at least a 10% reduction in PROMIS depression score. The treatment provided by system 10 can achieve a reduction in the PROMIS depression score of 2.27±3.75. The treatment provided by system 10 can achieve at least a 10% reduction in PROMIS anxiety score. The treatment provided by system 10 can achieve a reduction in the PROMIS anxiety score of 2.87±6.21.

### Study S5

As described herein, system 10 of the present inventive concepts can be used to treat depression of a patient. Severe forms of depression have been linked to excessive subcallosal cingulate (SCC) activity. Stimulation of SCC with surgically implanted electrodes can alleviate depression, but current noninvasive techniques cannot directly and selectively modulate deep targets. Use of system 10 provides a novel noninvasive neuromodulation approach that can deliver low-intensity focused ultrasonic waves to the SCC. In a study, study S5, twenty-two patients with treatment-resistant depression participated in a randomized, double-blind, sham-controlled study. Ultrasonic stimulation was delivered using system 10 to bilateral SCC of each patient's brain during concurrent functional MRI (e.g., via an imaging device 800 comprising an MRI) to quantify target engagement. In some embodiments, system 10 can be configured to deliver the ultrasound without concurrent functional MRI. In study S5, mood state was measured with the Sadness subscale of the Positive and Negative Affect Schedule before and after 40 minutes of real (also referred to as “active”) or sham SCC stimulation. Change in depression severity was measured with the 6-item Hamilton Depression Rating Scale (HDRS-6) at 24 hours and 7 days. Results: Functional MRI demonstrated a target-specific decrease in SCC activity during stimulation (p=0.028, n=16). In 8 of 16 participants, SCC neuromodulation was detectable at the individual-subject level with a single 10-minute scan (p<0.05, small-volume-correction). Mood and depression scores improved more with real than with sham stimulation. In the per-protocol sample (n=19), real stimulation was superior to sham for HDRS-6 at 24 hours and for Sadness (both p<0.05, d>1). Non-significant trends were found in the intent-to-treat sample. In study S5, ultrasonic stimulation using system 10 was shown to modulate SCC activity and rapidly reduce depressive symptoms.

Deep neural circuits are implicated in the pathophysiology of numerous psychiatric illnesses including mood disorders, anxiety disorders, and addictions. Current treatments for these illnesses are often ineffective, but better therapeutic approaches may be possible through specific and precise modulation of the activity of deep neural targets. Severe depression, for example, has been linked to excessive activity of the subcallosal cingulate cortex (SCC), a limbic region situated ventral to the corpus callosum. Functional imaging studies have shown that the SCC is hyperactive in depressed individuals and interventional studies indicate that disruption of SCC activity by deep brain stimulation can relieve depressive symptoms.

Current approaches to deep brain stimulation, however, require surgical implantation of stimulation leads, which carry considerable risks. The high risk-benefit ratio and high costs of surgical interventions limit the spectrum of individuals who could benefit from invasive approaches. Current noninvasive neuromodulation modalities, on the other hand, are limited in other ways. Transcranial magnetic and electric stimulation cannot directly and selectively modulate deep structures like the SCC due to fundamental physical limitations of electromagnetic fields. This lack of selectivity leads to limited effectiveness and excessive adverse effects.

System 10 addresses these limitations. As described herein, system 10 can be used to modulate the SCC and other deep brain targets noninvasively and in a controlled manner. Also as described herein, treatment device 100 of system 10 can comprise ultrasound transducer arrays (e.g., arrays of energy delivery elements 155 comprising ultrasound transducers) configured to focus low-intensity ultrasonic waves into deep brain targets through the intact skull and scalp. Critically, system 10 can be configured to measure and compensate for the substantial aberrations of ultrasound by the human head and/or other obstacles, thus delivering into the target a controlled, deterministic, and safe ultrasound intensity.

In study S5, system 10 was used to treat a cohort of participants (also referred to as “patients” or “subjects” herein) suffering from treatment-resistant depression using a randomized, blinded, sham-controlled study design. Ultrasonic stimulation was delivered by system 10 to the SCC using individualized MRI guidance, and the neural effects of stimulation were quantified using concurrent functional MRI. The two parallel objectives of the study were (1) to demonstrate that ultrasonic stimulation engages the SCC target and (2) to characterize the immediate mood effects and tolerability of this stimulation. System 10 was used to “deactivate” (e.g., reduce the activation of) the SCC and improve mood and depressive symptoms for the patients treated with active stimulation.

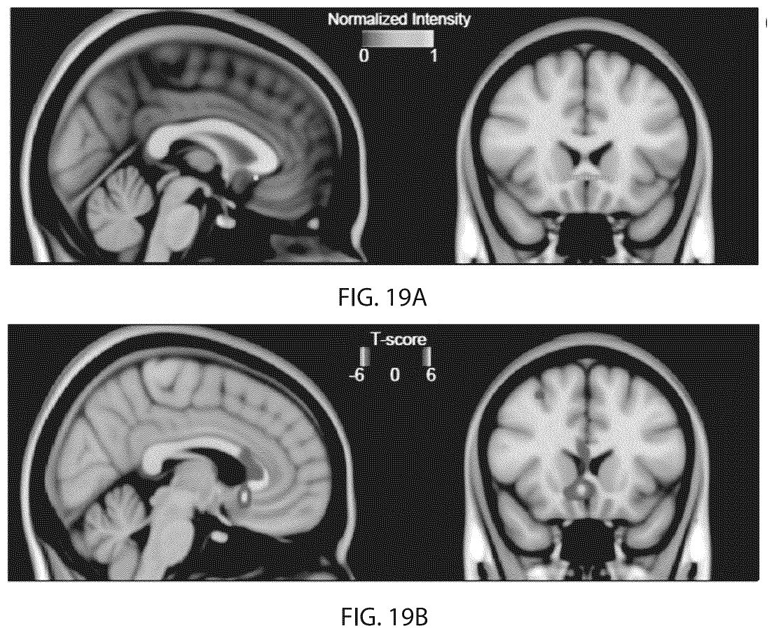

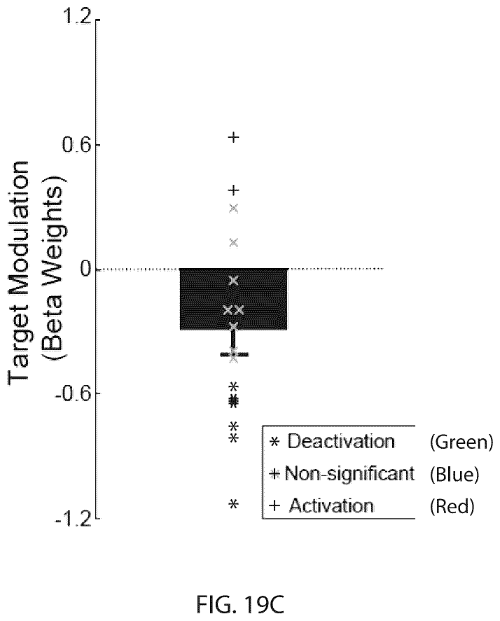

FIGS. 19 through 28 are related to study S5.

FIGS. 19A-B shows two images of a brain illustrating ultrasonic targeting and neuromodulation achieved using system 10. FIG. 19C is a graph of target modulation. In FIG. 19A, the ultrasound field produced at focus overlaid on sagittal and coronal images is illustrated. The dimensions of the focus are 20.4 mm by 2.4 mm by 3.6 mm (MNI space). In FIG. 19B, target engagement in an individual subject is illustrated, assessed using concurrent BOLD imaging. The SCC was selectively deactivated: peak coordinates=[4, 20, −6], whole-brain FWE-corrected p<0.001. Display thresholds were: t<−4, t>4, cluster size >50 voxels. In FIG. 19C, modulation of the targeted SCC region across the cohort (n=16) is illustrated. Each symbol represents the BOLD response extracted from SCC for an individual subject. The bar shows the mean±SEM response: t(15)=−2.43, p=0.028, one-sample two-tailed t-test. The symbol colors indicate individual-subject results from small volume-correction analyses (see Table 7). Statistically significant deactivation was observed at the individual-subject level in 6 subjects (green). Two subjects showed a significant activation (red). In 8 subjects (blue), the modulation was not significant at the individual level.

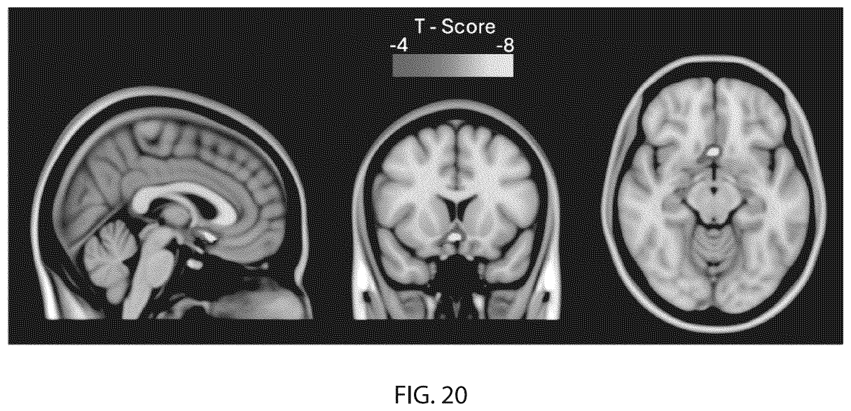

FIG. 20 shows three images of a brain illustrating selective deactivation of the SCC achieved using system 10. Group analysis performed on the 12 subjects that showed a deactivation of the SCC during stimulation is illustrated. The corresponding negative beta weights are shown in FIG. 19C. No other brain regions were significantly deactivated.

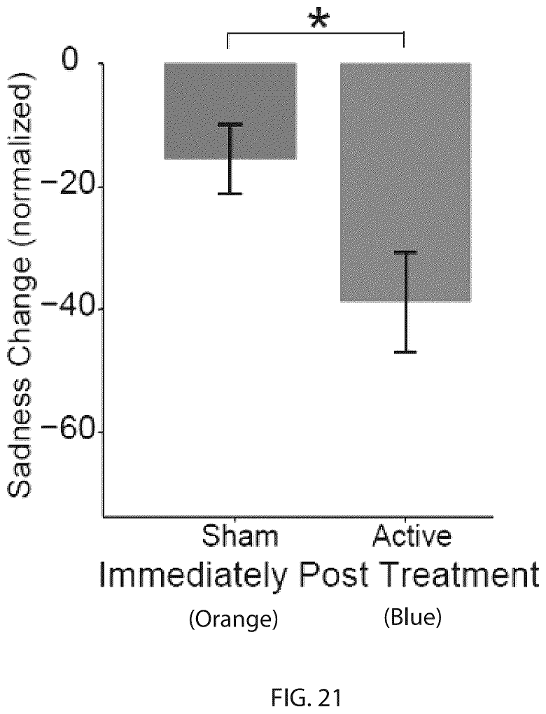

FIG. 21 shows a bar chart representing improvements in mood following ultrasonic modulation of the SCC using system 10. FIG. 21 illustrates an immediate change in PANAS-X Sadness score with treatment for sham stimulation (n=10) versus active stimulation (n=8) for the per-protocol sample. The mean percentage change in Sadness was-63% in the active group versus-47% in the sham group. Between-group standardized effect size d=−1.15 (95%-confidence interval=[−2.24, −0.07]). In the figure, * represents p<0.05, and error bars denote SEM.

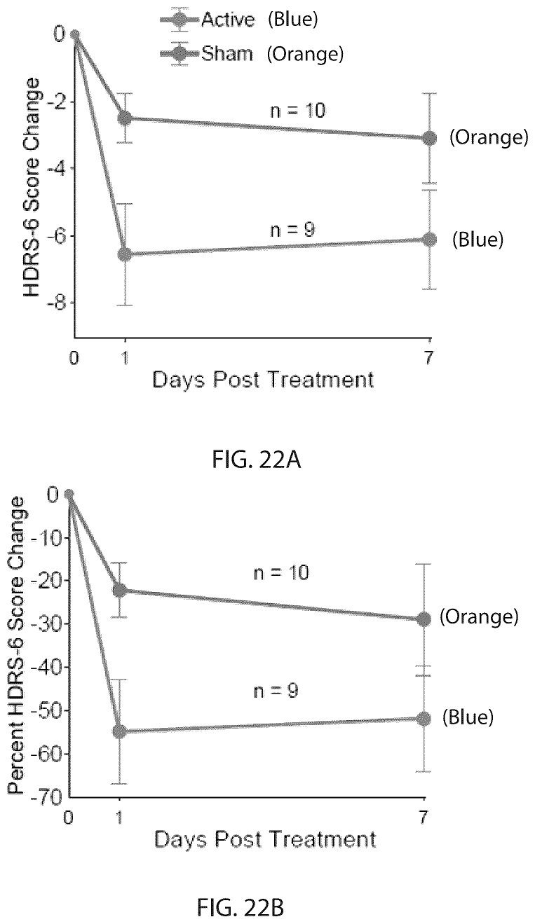

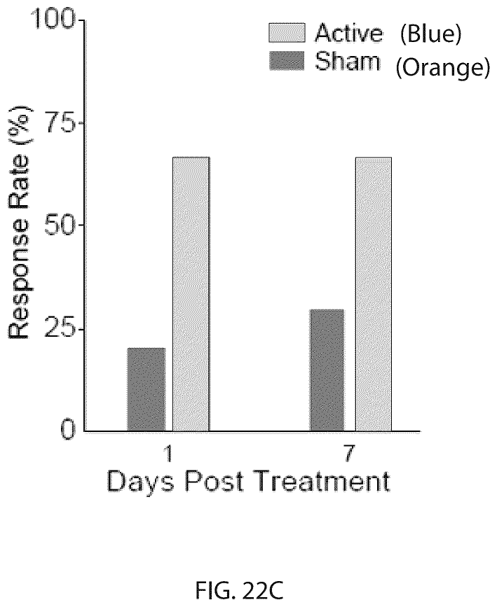

FIGS. 22A-C are two graphs and a bar chart showing the improvements in depressive symptoms following ultrasonic stimulation of the SCC achieved using system 10. FIG. 22A shows the change in the 6-item Hamilton Depression Rating Scale (HDRS-6) score 24 hours and 7 days post-treatment for the per-protocol sample. Also shown are between-group standardized effect size d=−1.078 (95%−confidence interval=[−2.11, −0.04]) at 24 hours and −0.59 (95%−confidence interval=[−1.59, 0.40]) at 7 days. FIG. 22B shows the mean percentage changes in HDRS-6 scores at 1 and 7 days: −55% and −52% in the active group and −22% and −29% in the sham group. FIG. 22C shows the proportion of subjects who experienced at least a 50% reduction in HDRS-6 score, shown at 24 hours and 7 days for the active group (n=9) and sham group (n=10).

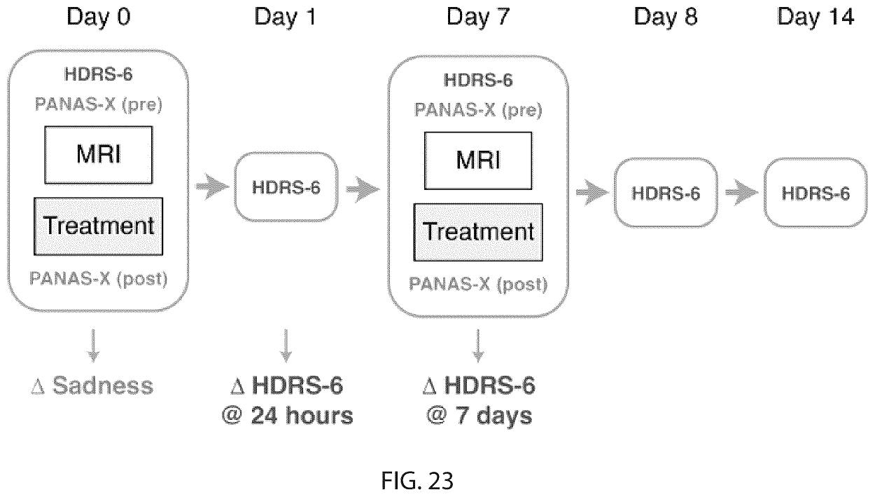

FIG. 23 is a flow chart representing the clinical trial design of study S5. As described hereinabove, study S5 comprised a double-blind, randomized, sham-controlled, cross-over design. Clinical evaluation was performed at the baseline visit (not shown). Approximately 1 week later the participant returned for the first stimulation visit (Day 0), where they were randomized 1:1 to the real-stimulation or sham-stimulation arm. Pre-treatment scales were collected: 6-item Hamilton Depression Rating Scale (HDRS-6) and expanded Positive and Negative Affect Schedule (PANAS-X). Each subject then participated in a 1-hour MRI session followed immediately by a 1-hour treatment session. A post-treatment PANAS-X was completed immediately after the treatment session. The HDRS-6 was repeated 24 hours later (Day 1). Participants returned 6 days later (Day 7) when their treatment assignment crossed over to sham or real, and the procedures of Day 0 were repeated. The HDRS-6 was repeated 24 hours and 7 days later (Day 8 and Day 14). The primary efficacy outcomes, shown at bottom of the figure, were changes in PANAS-X Sadness score on Day 0, change in HDRS-6 score from Day 0 to Day 1, and change in HDRS-6 score from Day 0 to Day 7.

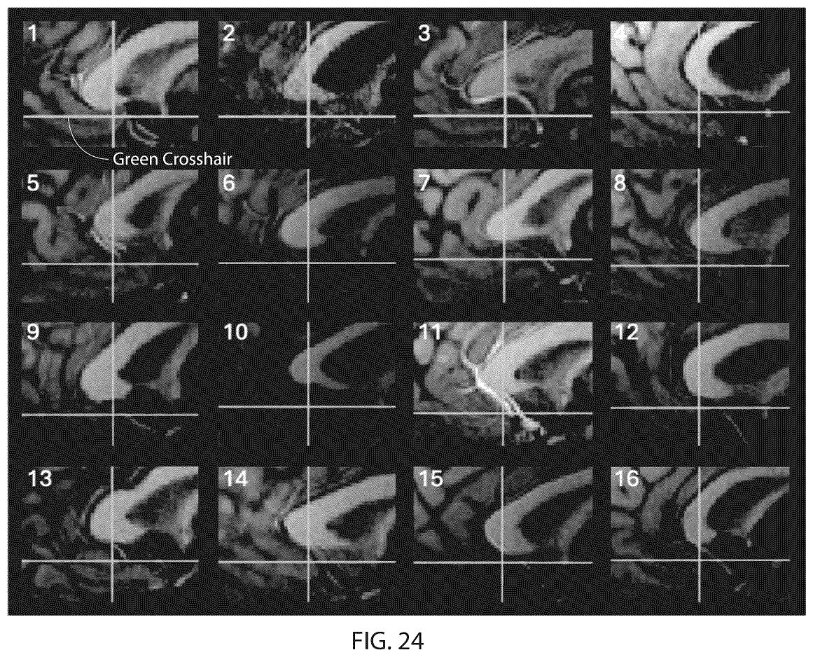

FIG. 24 is a set of brain images showing individual subcallosal cingulate ultrasound targets. In the 16 images shown, green crosshairs are overlaid on a sagittal T1-weighted image from each subject. The ultrasound focus provided by system 10 was centered on the midline and extended into subcallosal cingulate cortex bilaterally.

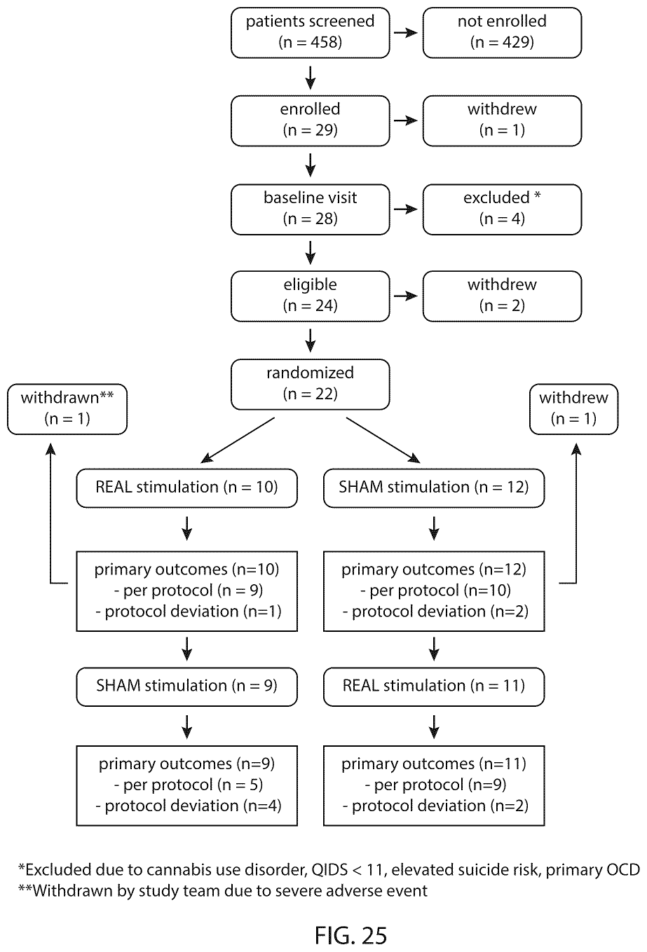

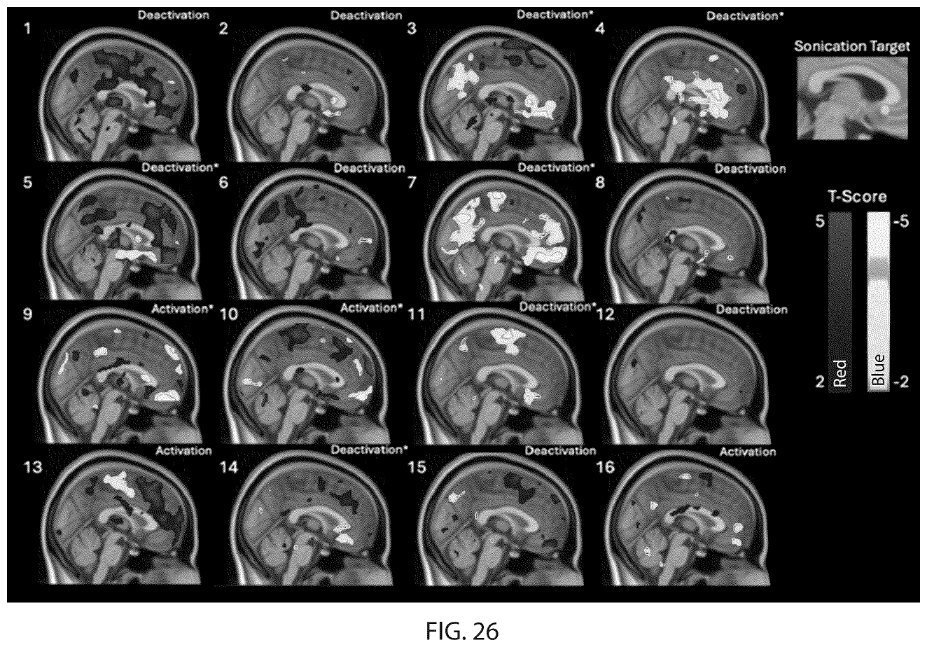

FIG. 25 is a consort diagram of study participants of study S5. The intent-to-treat sample included all randomized subjects. Prior to cross-over, the per-protocol sample excluded 2 subjects who received real stimulation during MRI at the sham visit and 1 subject who had received real stimulation before randomization. After cross-over, the per-protocol sample excluded 3 additional subjects who received real stimulation during MRI at the sham visit.

FIG. 26 is a set of brain images representing distributed neuromodulation during subcallosal cingulate sonication achieved using system 10. Shown are SCC sonication that elicited diverse patterns of activation and deactivation in distributed brain areas. The SCC target is shown in the upper right inset. Each panel represents an individual patient. The individual-level direction and significance of SCC neuromodulation (see Table 7) is noted at the top of each panel. In the figure, * represents significance at the individual-subject level (p<0.05). Areas of activation (red) and deactivation (blue) are overlaid on a sagittal image. Display threshold: t<−2 and t>2.

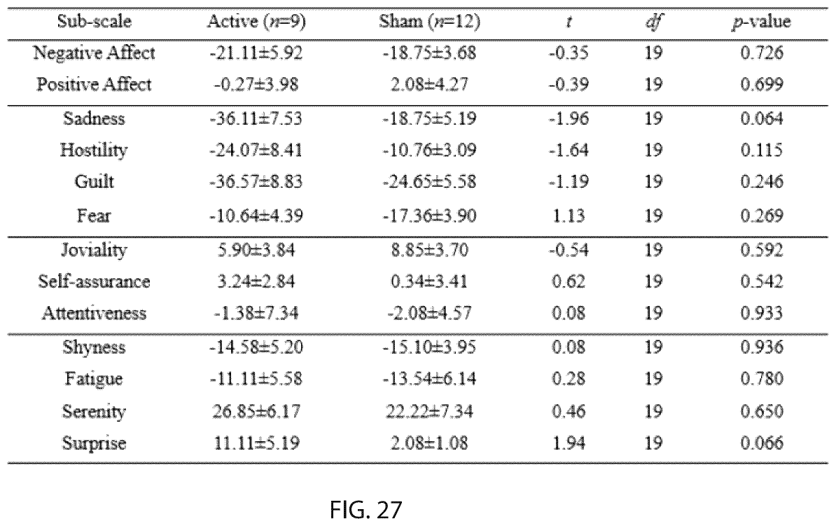

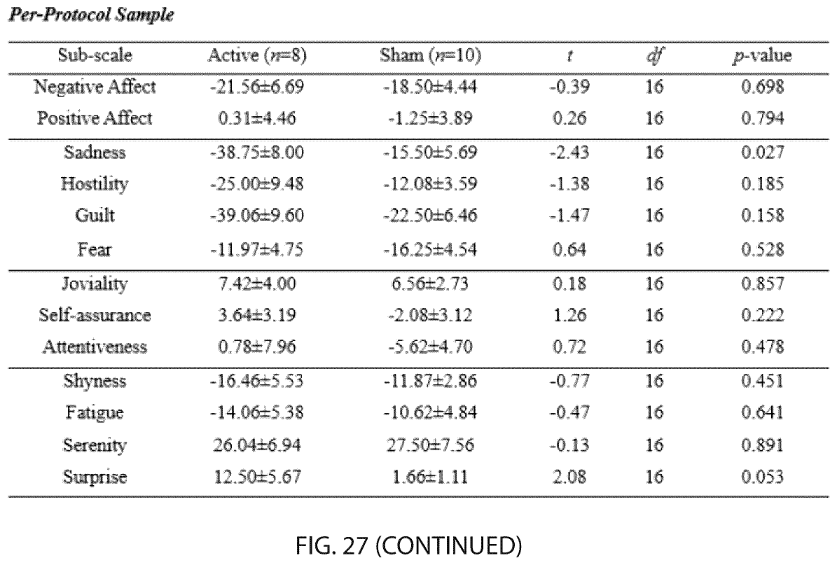

FIG. 27 is a table representing score changes for the expanded Positive and Negative Affect Schedule of study S5. Values shown are presented as mean and standard error of the mean. Two-sample t-tests were used. Data is missing for one subject who received active stimulation. Sadness was the pre-specified primary outcome measure.

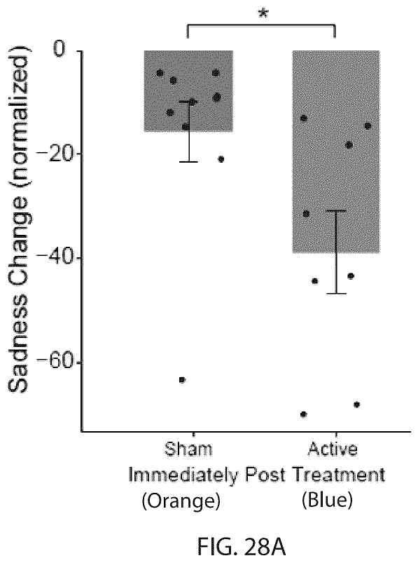

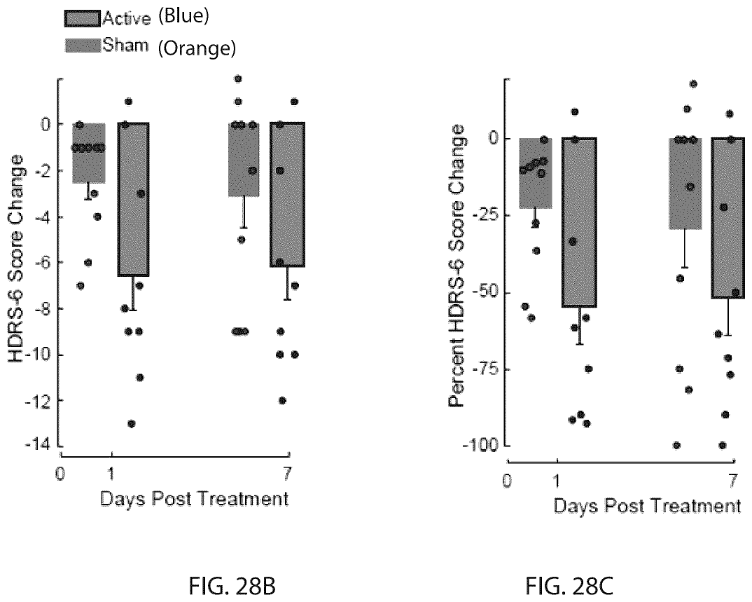

FIGS. 28A-C are three bar charts representing PANAS-X Sadness change, HRRS-6 score change, and percent HDRS-6 score change, respectively, for individual subjects treated per protocol. In FIG. 28A, the immediate change in PANAS-X Sadness scores is illustrated. In FIG. 28B, the change in HDRS-6 score at 24 hours and 7 days is illustrated. In FIG. 28C, the percent change in HDRS-6 score is illustrated.

Table 5 comprises a list of inclusion and exclusion criteria for study S5.

TABLE 5
Inclusion Criteria

| | |
|---|---|
| 1. | Age 18-65, any gender |
| 2. | Primary diagnosis of major depressive disorder or bipolar disorder |
| 3. | Current moderate-to-severe depressive episode, without psychotic |
| | features, lasting at least 2 months |
| 4. | Self-rated 16-item Quick Inventory of Depressive Symptomatology |
| | (QIDS) total score > 10 |
| 5. | Stated willingness to comply with all study procedures and avoid |
| | changes to psychiatric treatments (medications, psychotherapy) for |
| | the duration of the study |
| 6. | For females of reproductive potential: negative pregnancy test or |
| | use of highly effective contraception for at least 1 month prior to |
| | baseline; agreement to use such a method throughout the study |
| 7. | Capacity to provide informed consent: provision of a signed and |
| | dated consent form |

Exclusion Criteria

| | |
|---|---|
| 1. | History of serious brain injury or other neurologic disorder |
| 2. | Poorly managed general medical condition |
| 3. | Pregnant or breast feeding |
| 4. | Implanted device in the head or neck |
| 5. | MRI intolerance or contraindication |
| 6. | Brain stimulation (e.g., ECT, TMS, VNS) in the past month |
| 7. | Clinically significant suicidal ideation in the past month. |
| 8. | Lifetime history of a serious suicide attempt |
| 9. | Moderate-to-severe substance use disorder (past 3 months) |
| 10. | Obsessive compulsive disorder, primary diagnosis (past month) |
| 11. | Posttraumatic stress disorder, primary diagnosis (past month) |
| 12. | Schizophrenia-spectrum disorder (lifetime) |
| 13. | Neurocognitive disorder (past year) |
| 14. | Personality disorder as a current focus of treatment |
| 15. | Clinically inappropriate for participation in the study as |
| | determined by the study team |

Table 6 includes baseline demographic and clinical features for the intent-to-treat sample of study S5. Diagnoses were determined with the Mini International Neuropsychiatric Interview, version 7.0. Two participants with bipolar disorder had a history of sub-threshold manic symptoms that did not meet criteria for bipolar 1 or 2 disorder. Only clearly documented failed antidepressant medication trials in the past 2 years were included. No significant differences were found between real and sham groups (all p>0.05).

TABLE 6

| | | |
|---|---|---|
| Real | Sham | |
| Stimulation | Stimulation | All Subjects |
| (n = 10) | (n = 12) | (n = 22) |

Demographics

| | | | |
|---|---|---|---|
| Age, years, mean (SD) | 37.37 (11.85) | 41.23 (8.41) | 39.47 (10.05) |
| Female Gender, n (%) | 6 (60%) | 8 (67%) | 14 (64%) |
| Non-Hispanic White, n (%) | 7 (70%) | 10 (83%) | 17 (77%) |
| Education, years, mean (SD) | 15.9 (1.10) | 15.83 (1.64) | 15.86 (1.39) |

Diagnoses and Comorbidities
Primary DSM-5 diagnosis

| | | | |
|---|---|---|---|
| Major Depressive Disorder, n (%) | 9 (90%) | 11 (92%) | 30 (91%) |
| Other: Bipolar Disorder, n (%) | 1 (10%) | 1 (8%) | 2 (9%) |
| Generalized Anxiety Disorder, n (%) | 4 (40%) | 7 (58%) | 11 (50%) |
| Panic Disorder, n (%) | 2 (20%) | 4 (33%) | 6 (27%) |
| Agoraphobia, n (%) | 3 (30%) | 1 (8%) | 4 (18%) |
| Social Anxiety Disorder, n (%) | 4 (40%) | 5 (42%) | 9 (41%) |
| Obsessive Compulsive Disorder, n (%) | 2 (20%) | 1 (8%) | 3 (14%) |
| Posttraumatic Stress Disorder, n (%) | 4 (40%) | 2 (17%) | 6 (27%) |
| Alcohol Use Disorder (mild), n (%) | 0 (0%) | 1 (8%) | 1 (5%) |
| Other Substance Use Disorder (mild), n (%) | 2 (20%) | 0 (0%) | 2 (9%) |
| Lifetime History of Psychosis, n (%) | 0 (0%) | 1 (8%) | 1 (5%) |

Severity Scales

| | | | |
|---|---|---|---|
| HDRS-6, mean (SD) | 12.00 (2.91) | 11.42 (1.38) | 11.68 (2.17) |
| IDS-SR, mean (SD) | 43.90 (9.80) | 44.42 (9.62) | 44.18 (9.47) |
| QIDS-SR, mean (SD) | 17.10 (3.28) | 18.33 (3.11) | 17.77 (3.18) |
| YMRS, mean (SD) | 1.10 (1.20) | 1.00 (0.83) | 1.05 (1.00) |
| GAD-7, man (SD) | 12.60 (5.80) | 10.00 (5.38) | 11.18 (5.59) |

Chronicity and Resistance

| | | | |
|---|---|---|---|
| Age of Onset, years, mean (SD) | 17.70 (5.25) | 14.33 (5.82) | 15.86 (5.70) |
| Duration of Current Episode, months, median (IQR) | 13.00 (12.00) | 24.00 (27.50) | 20.00 (23.25) |
| Chronic Episode (>24 months). n (%) | 4 (40%) | 6 (50%) | 10 (45%) |
| Failed Antidepressant Trials, Current Episode, mean (SD) | 1.60 (0.70) | 1.42 (1.24) | 1.50 (1.01) |
| Maudsley Staging Method, mean (SE) | 7.40 (1.58) | 7.42 (1.38) | 7.41 (1.44) |

METHODS AND MATERIALS—Study Design and Participants: Eligible individuals were adults (age 18-65 years) with a primary DSM-5 diagnosis of major depressive disorder or bipolar disorder and a current moderate-to-severe depressive episode without psychotic features lasting at least 2 months (see Table 5 for full inclusion/exclusion criteria). Study S5 incorporated a double-blind, randomized, sham-controlled, cross-over design (reference FIG. 23). Clinical evaluation was performed at the baseline visit, and approximately 1 week later the patient returned for the first stimulation visit, where they were randomized 1:1 to the real-stimulation or sham-stimulation arm. At the first stimulation visit, each patient participated in a 1-hour MRI session followed immediately by a 1-hour treatment session. Seven days later, each patient returned for the second stimulation visit, where patients crossed over from real to sham, or sham to real. At the second stimulation visit, the 1-hour MRI session and 1-hour treatment session were repeated. Symptoms were assessed at the start and end of each stimulation visit, and 24 hours and 7 days following each stimulation visit.

METHODS AND MATERIALS—System 10: In study S4, treatment device 100 of system 10 included two ultrasound transducer phased arrays that were situated in a frame over the left and right sides of the head of the patient, such as via housing 110 and/or other system 10 components as described herein. Acoustic coupling gels were placed between each array and the head, and a thermoplastic mask (e.g., mask 700a described herein) was individually fit to the patient's head to minimize movement relative to the frame and transducer arrays. A transmit-receive scan was performed between the two arrays to measure the acoustic distortion caused by the head and coupling, and an algorithm calculated phase and amplitude adjustments at each transducer element to compensate for the distortion. MRI (e.g., via imaging device 800) was performed with the ultrasound transducer arrays locked in place and the arrays were co-registered to the individual's brain anatomy using fiducial markers on the device that were visible in the MRI. The treatment device 100 created a sonication focus that extended 20.4 mm by 2.4 mm by 3.6 mm (x, y, z dimensions in Montreal Neurological Institute (MNI) space). The focus was moved to the desired target programmatically without moving the treatment device 100 or patient. To assure safety, ultrasound intensities were always delivered below the FDA 510(k) Track 3 guidelines for diagnostic ultrasound (peak intensity less than 190 W/cm², time-averaged intensity less than 720 mW/cm², mechanical index less than 1.9).

METHODS AND MATERIALS—Targeting and Measurement of Target Engagement: The individual's T1-weighted image was used to guide ultrasound targeting. Blood oxygenation level dependent (BOLD) imaging was used to measure target engagement. Details of MRI acquisition are provided in the Supplementary Methods section hereinbelow. The center of the sonication focus was positioned on the midline, spanning left and right SCC, in order to approximate bilateral targets previously described for invasive DBS. Individual targets are shown in FIG. 24. Ultrasound was delivered with an amplitude of 1 MPa at target (31.1 W/cm²; following skull correction), 30 ms burst duration containing pulses of 5 ms on and 5 ms off, separated by 1.4-second burst intervals, for 60 seconds. These stimulus parameters were expected to cause net inhibitory effects. Neuromodulation was quantified using concurrent (online) BOLD imaging with a 10-minute block-design paradigm consisting of five 1-minute rest epochs (no sonication) interleaved with five 1-minute epochs of active sonication. Continuous white noise was played throughout the 10-minute session via earbuds with the goal of masking any potential auditory effects caused by the ultrasound. Because of technical difficulties during some BOLD imaging sessions, in several cases it was necessary to deviate from protocol and repeat real stimulation during functional MRI at the second stimulation session even though treatment assignment had crossed over from real to sham (see Supplemental Methods hereinbelow). Usable BOLD data were not obtainable from 5 of 21 participants who received sonication during MRI due to technical problems, so the final data set reported here includes a total of 16 subjects.

METHODS AND MATERIALS—Randomization and Blinding: Participants were randomized to real (active) or sham (placebo) groups when they arrived for the first stimulation visit. The allocation sequence was created with a random-number generator using a block size of 6. A sequence of envelopes was prepared prior to the trial with notes designating “active” or “sham” such that no one knew the sequence. The system 10 operator was necessarily unblinded to allocation at the first stimulation visit. All other staff, participants, and clinical raters remained blinded. Treatment allocation was disclosed to participants following the HDRS-6 rating 7 days after the second stimulation visit.

The effectiveness of blinding was assessed by asking a subset of 13 participants to rate on a 0-100 scale their best guess about which intervention they received: 0 represented certainty it was sham stimulation, 100 certainty it was real stimulation, and 50 complete uncertainty. Ratings were collected at the end of the first stimulation visit.

METHODS AND MATERIALS—Real and Sham Treatment Sessions: After the MRI imaging, stimulation was delivered outside the MRI scanner during a single 1-hour treatment session including 39-41 minutes (cumulative) of real or sham sonication. Three midline targets within the SCC region were stimulated sequentially, with the goal of maximizing changes in mood symptoms. In addition to the original target that was stimulated during concurrent functional MRI, two targets approximately 4 mm anterior and 4 mm posterior were defined on the individual MRI. The targets were then stimulated for equal duration, in random order, over a 1-hour session. Ultrasound was delivered to each target with an amplitude of 1 MPa (31.1 W/cm²; following skull correction), 30 ms burst duration containing pulses of 5 ms on and 5 ms off, separated by 1.4-second or 0.7-second burst interval. Overall, the treatment session consisted of three stimulation blocks (A, B, C). Block A contained 3-5 one-minute sonications to test the tolerability of each target with 1.4-second burst intervals. Block B contained 6 three-minute sonications with 1.4-second burst intervals. Block C contained 6 three-minute sonications with 0.7-second burst intervals.

To mask the faint vibratory percepts sometimes experienced with ultrasound stimulus delivery, patients wore earbuds and received identical auditory stimuli during both active and sham interventions. White noise was combined with audio recordings of ultrasound pulses from the arrays, and auditory stimuli were timed to coincide with delivery of ultrasound stimulation.

METHODS AND MATERIALS—Clinical Outcomes Measures: Two co-primary efficacy measures were pre-specified. The Sadness sub-scale of the expanded Positive and Negative Affect Schedule (PANAS-X) quantified immediate change in mood state, and the HDRS-6 measured changes in depressive symptom severity at 24 hours and 7 days (reference FIG. 23). The PANAS-X is a reliable, validated, 60-item, self-report of mood state. This scale was administered at the beginning and end of each stimulation visit using a “right now” time frame. The Sadness subscale includes 5 items (sad, blue, downhearted, alone, lonely), each rated on a 5-point scale. Each PANAS-X subscale was rescaled to a 0-100 range and change (post-minus pre-stimulation) was calculated. The HDRS-6 is an abbreviated version of the original 17-item instrument that includes core symptoms of depression (depressed mood, feelings of guilt, work and activities, retardation, psychic anxiety, general somatic symptoms). This scale strongly correlates with longer versions of the HDRS, is sensitive to change, and is usable with brief time frames allowing measurement of rapid effects. The HDRS-6 was administered by a blinded psychiatrist or senior psychiatry resident at the beginning of each stimulation visit (e.g., before stimulation) using a 7-day time frame, and repeated 24 hours and 7 days after the visit using 24-hour and 7-day time frames. Tolerability and safety were assessed at each visit through collection of spontaneously reported adverse events, the General Assessment of Side Effects (GASE) scale, the YMRS, and the C-SSRS. Secondary outcomes are described in the Supplementary Material hereinbelow.

METHODS AND MATERIALS—Functional MRI Analysis: The functional MRI processing pipeline is described in the Supplementary Methods. The primary analysis of target engagement used a region of interest (ROI) corresponding to the SCC volume where ultrasound was delivered. The ROI was constructed by horizontally concatenating 3 spheres of 4-mm radius around MNI coordinates [0,20,−8] to create an ellipsoid of radius 4 mm (y and z dimensions) and length 12 mm (x dimension). The volume of the ROI was thus restricted to grey matter and inflated beyond the volume of the sonication focus approximately 4-fold to allow for individual differences in targeting and displacement of peaks in statistical parametric maps. Target engagement was evaluated for each patient with a subject-level GLM family-wise-error small-volume-correction (FWE-SVC) analysis restricted to the SCC ROI. In addition, collapsed GLM beta-weights were extracted from the ROI for each participant from a second-level group model, contrasting active stimulation blocks and rest blocks (On>Off t-test). Target engagement at the group level was evaluated using a one-sample t-test on these extracted beta-weights. Neural responses of brain areas beyond the SCC (off-target effects) were evaluated with whole-brain, voxel-wise analyses. Two whole-brain directional t-tests compared rest blocks to sonication blocks (Off>On and On>Off t-tests) to reveal brain regions that were activated or deactivated during sonication.

METHODS AND MATERIALS—Analysis of Clinical Outcomes: Change in PANAS-X Sadness (post-minus pre-stimulation) at the first stimulation visit was calculated for each patient and the difference between treatment groups (real versus sham) was evaluated using a two-sample t-test. Similarly, changes in HDRS-6 score 24 hours and 7 days after the first stimulation visit were calculated, and two sample t-tests were applied to test for group differences at each time point.

The intention-to-treat sample included all 22 participants who were randomized. The per-protocol sample (n=19) excluded 3 patients who received stimulation other than as intended in the pre-specified protocol. One patient had received active stimulation during MRI under a different protocol prior to being randomized to the real arm; one received active stimulation during MRI despite being randomized to the sham arm; and one inadvertently received several minutes of active stimulation during the sham treatment session.

RESULTS—Characteristics of Participants: Twenty-nine adults with treatment-resistant depression enrolled in the study and 22 were randomized (see CONSORT diagram, FIG. 25). Ten were assigned real stimulation at the first session and 12 sham stimulation. Demographic and clinical characteristic were comparable for real-versus sham-stimulation groups (all p>0.05, see Table 6). Twenty participants returned for a second stimulation visit to cross over to the other condition (sham or real).

Table 7 is a table of information related to Individual-Subject Analysis of Target Engagement. A region-of-interest analysis was performed for each participant using an SPM12 general linear model with small-volume correction (SVC). The volume of interest was the sonicated region within the subcallosal cingulate. In Table 7, asterisks indicate statistically significant modulation of the target at the individual level.

TABLE 7

| | Modulation Direction | FWEc p-value | T-Score | Z-Score |
|---|---|---|---|---|
| Subject 1 | Deactivation | 0.156 | 2.21 | 2.2 |
| Subject 2 | Deactivation | 0.103 | 2.48 | 2.46 |
| Subject 3 | Deactivation* | <0.001 | 4.38 | 4.3 |
| Subject 4 | Deactivation* | <0.001 | 5.63 | 5.48 |
| Subject 5 | Deactivation* | 0.002 | 3.93 | 3.87 |
| Subject 6 | Deactivation | 0.158 | 2.27 | 2.26 |
| Subject 7 | Deactivation* | 0.006 | 3.57 | 3.53 |
| Subject 8 | Deactivation | 0.582 | 1.19 | 1.23 |
| Subject 9 | Activation* | <0.001 | 4.37 | 4.3 |
| Subject 10 | Activation* | 0.002 | 3.86 | 3.8 |
| Subject 11 | Deactivation* | 0.001 | 4.25 | 4.18 |
| Subject 12 | Deactivation | 0.349 | 1.59 | 1.6 |
| Subject 13 | Activation | 0.342 | 1.76 | 1.76 |
| Subject 14 | Deactivation* | 0.031 | 2.98 | 2.95 |
| Subject 15 | Deactivation | 0.332 | 1.78 | 1.78 |
| Subject 16 | Activation | 0.559 | 1.22 | 1.25 |

RESULTS—Target Engagement: Individual-level analyses showed statistically significant neuromodulation in the targeted SCC region in 8 of 16 subjects (p<0.05, FWE-SVC) based on a single 10-minute BOLD imaging session (see Table 7). For 6 subjects, the expected decrease in BOLD signal (i.e., deactivation) was detected. An example of the SCC deactivation is shown in FIGS. 19A-B. Eight participants showed no significant modulation and 2 showed significant activation of the SCC. At the group level, the average effect of the SCC modulation was deactivation: beta-weights extracted from the SCC region of interest (reference FIG. 19C) were significantly less than zero across the cohort (t(15)=−2.43, p=0.028, one-sample two-tailed t-test).

RESULTS—Brain-Wide Effects: To evaluate the broader effects of SCC modulation on brain networks, the effects across the brain in each subject were evaluated. Diverse patterns of activation and deactivation were observed in distributed brain regions (reference FIG. 26). At the group level, whole-brain analysis revealed no consistent deactivation beyond the SCC (p>0.05, false discovery rate corrected). However, activation (e.g., an increase in activity) was detected at a group level in the left ventrolateral prefrontal cortex and right superior temporal gyrus (reference Table 8).

Responses in distributed brain areas may depend on the polarity of the SCC modulation. Therefore, the subset of the 12 subjects for whom sonication deactivated the SCC was also analyzed. Significant deactivation was found only in the SCC (reference FIG. 20). Activation was detected in the left ventrolateral prefrontal cortex and bilateral temporal cortex (reference Table 8), in line with the findings for the full cohort.

TABLE 8

| | Cluster | Cluster | Signal | Signal | MNI Coordinates | |
|---|---|---|---|---|---|---|
| Sample | Size(Voxels) | p(FDR-corr) | T-Score | p(unc) | x, y, z [symbol] mm [symbol] | Broadman Areas |
| Whole | 203 | 0.045 | 6.20 | <0.001 | −44, 36, −8 | (47) Left Ventral |
| Sample | | | | | | Lateral Prefrontal |
| (N = 16) | | | | | | Cortex |
| | 231 | 0.045 | 4.97 | <0.001 | 60, −40, 10 | (22) Right |
| | | | | | | Supramarginal |
| | | | | | | Temporal Gyrus |
| Sub- | 227 | 0.013 | 7.23 | <0.001 | −42, 36, −8 | (47) Left Ventral |
| Sample | | | | | | Lateral Prefrontal |
| (N = 12) | | | | | | Cortex |
| | 138 | 0.042 | 6.08 | <0.001 | −54, −26, 4 | (41) Left Primary |
| | | | | | | Auditory Cortex |
| | 123 | 0.042 | 5.90 | <0.001 | 56, −38, 12 | (22) Right |
| | | | | | | Supramarginal |
| | | | | | | Temporal Gyrus |

[symbol]

indicates data missing or illegible when filed

Table 8 is a table of data related to brain regions activated by the subcallosal cingulate sonication performed in study S5. Results of SPM12 general linear models for the ON>OFF contrast are shown.

RESULTS—Changes in Mood and Depression: The primary efficacy endpoints were change in PANAS-X Sadness immediately following stimulation, and change in HDRS-6 score 24 hours and 7 days following the stimulation visit.

In the per-protocol sample (n=19), group differences for Sadness and HDRS-6 at 24 hours were statistically significant (Sadness: p=0.027, t(16)=−2.43, d=−1.15; HDRS-6 at 24 hours: p=0.031, t(17)=−2.35, d=−1.08; HDRS-6 at 7 days: p=0.22, t(17)=−1.29, d=−0.59), as shown in FIGS. 21, 22A-C, and 28. The rate of response (improvement ≥50%) for sham versus active stimulation was 20% versus 67% at 24 hours, and 30% versus 67% at 7 days (reference FIGS. 22A-C).

For the intent-to-treat sample (n=22), scores decreased more in the real-stimulation group compared with the sham-stimulation group, but group differences did not reach a p<0.05 significance for Sadness (p=0.064, t(19)=−1.96, Cohen's d=−0.87), HDRS-6 at 24 hours (p=0.18, t(20)=−1.38, d=−0.59), or HDRS-6 at 7 days (p=0.45, t(20)=−0.77, d=−0.33).

RESULTS—Tolerability and Safety: During this cross-over study, 21 participants received real stimulation and 21 received sham stimulation. No serious adverse events (SAEs) occurred during the course of the study. No severe adverse events occurred during or immediately after stimulation. No subjects experienced mania or hypomania.

At follow-up visits 24 hours after each stimulation visit, self-reported side effects were collected with a standardized questionnaire (reference Table 9). The symptoms most commonly reported were depressed mood (real, 62%; sham 67%), headache (real, 57%; sham, 67%), and anxiety (real, 57%; sham 52%). Suicidal thoughts were reported by 29% of subjects after real stimulation and 24% of subjects after sham.

Two participants experienced a severe psychiatric adverse event with onset later than the 24-hour follow-up visit, both of which followed real stimulation. The first participant developed acute depression with suicidal ideation 3 days after stimulation and took an intentional overdose of medication that did not require medical intervention. The other subject developed rapid worsening of depression with suicidal ideation a few hours after the 24-hour follow-up visit. Both subjects had a history of similar mood swings in the past. Over the subsequent 2 weeks, depression improved and suicidal ideation resolved for both participants.

TABLE 9

| | [symbol] | | | | | [symbol] | | | | |
|---|---|---|---|---|---|---|---|---|---|---|
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] |

[symbol]

indicates data missing or illegible when filed

Table 9 is a summary of stimulation safety data. Twenty-four hours after ultrasonic stimulation via system 10, participants completed a standard clinical questionnaire that assessed a range of potential side effects. The data are shown separately for the active (left column) and sham (right column) stimulation.

RESULTS—Effectiveness of Blinding: At the end of the first stimulation visit, 13 participants were asked to guess which intervention they received on a 0-100 scale, 50 representing complete uncertainty. The mean (SD) rating was 48 (32) for the sham group and 64 (26) for the active group. Neither was significantly different from 50 (p=0.87 and p=0.30, one-sample two-tailed t-tests), and mean values for the two groups did not differ from each other (p=0.35, two-sample two-tailed t-test). This suggests that the blinding procedures used in this study were effective.

DISCUSSION: Study S5 included use of system 10 on a cohort of individuals with treatment resistant depression. In this study, system 10 included a treatment device 100 comprising a phased array device that delivered ultrasound into specified deep brain targets, while measuring and compensating for the severe aberrations of ultrasound by the head. It was found that transcranial delivery of low-intensity focused ultrasound to the SCC using system 10 can safely reduce SCC activity, elicit immediate mood improvement, and rapidly decrease depressive symptoms. This proof-of-principle demonstration suggests that this use of system 10 can be applied to not only depression, but also a range of other neuropsychiatric disorders.

As described herein, ultrasonic waves combine a unique triad of properties: noninvasiveness, depth penetration, and sharp focus. Compared to electromagnetic waves, sound waves have a small wavelength. Thanks to diffraction, the short wavelength enables relatively sharp focus at depth. Nonetheless, ultrasonic technology has been impeded by formidable barriers: the skull and hair, which attenuate and distort ultrasonic waves severely and unpredictably. The approach used in study S5 directly measures and compensates for these obstacles in each individual, thus delivering into specified targets of a patient's brain a controlled, deterministic ultrasound intensity, resulting in safe and effective neuromodulation.

In study S5, reduced activity in the targeted area via sonication of the SCC was confirmed for the patient group as a whole and for 6 subjects with statistically significant deactivation at the individual level (reference FIG. 19C). In some embodiments, system 10 can be configured to provide individualized optimization of targeting, such as to achieve highly consistent effects across subjects. A major advantage of system 10 over surgical approaches is that the ultrasound focus can be readily steered to one or more different targets without the need to move the treatment device 100 or patient. Furthermore, multiple targets can be defined and sonicated sequentially or near-simultaneously using system 10. The flexibility of this approach, along with the safety of repeated stimulation, lends itself well to individualized optimization. This flexible approach could also be applied to other targets investigated with surgical deep brain stimulation such as the medial forebrain bundle and the ventral striatum.

Deactivation of the SCC with transcranial ultrasound using system 10 of the present inventive concepts can cause an immediate decrease in sad mood and a rapid improvement of depressive symptoms among individuals with moderate-to-severe treatment resistant depression. The differences between active and sham treatment groups were greatest for participants treated per protocol, and for the immediate and 24-hour assessments, and durable antidepressant effects lasting 1 week or longer were observed for a subset of participants.

In summary, study S5 comprised a noninvasive targeted approach to modulate deep brain limbic circuits involved in treatment-resistant depression using system 10. As demonstrated by the data, system 10 can be used to deliver ultrasound to noninvasively modulate the SCC and rapidly improve mood and depressive symptoms. By virtue of its noninvasiveness, system 10 has the potential to benefit a broad spectrum of patients who suffer from depression.

Below is additional information related to study S5.

SUPPLEMENTARY METHODS—Participants: Inclusion and exclusion criteria are shown in Table 5. Eligibility was confirmed with a complete psychiatric and medical history as well as physical and neurologic examination. A psychiatrist administered the Mini International Neuropsychiatric Inventory (version 7.0), 6-item Hamilton Depression Rating Scale (HDRS-6), and Young Mania Rating Scale (YMRS). Participants completed the self-rated Inventory of Depressive Symptomatology (IDS-SR), 7-item Generalized Anxiety Disorder scale (GAD-7), Columbia Suicide Severity Rating Scale (C-SSRS) and an MRI safety screen.

SUPPLEMENTARY METHODS—MRI Acquisition: MRI was acquired with a Siemens VIDA 3-T system and a large flex coil. To guide targeting, a T1-weighted structural image was obtained using a magnetization-prepared radiofrequency pulses and rapid gradient-echo (MPRAGE) acquisition (ascending series, A-P phase encoding, TR 2.4 s, TE 2.26 ms, FA 8 degrees, FOV 256 mm, acquisition matrix 256, reconstruction matrix 256, in-plane resolution 1.0 mm by 1.0 mm, 192 slices, slice thickness 1.3 mm, bandwidth 200 Hz/pixel, echo spacing 6.84 ms). To measure target engagement, the blood oxygenation level dependent (BOLD) signal was measured using T2*-weighted functional imaging (interleaved series, P-A phase encoding, TR 2.0 s, TE 33 ms, FA 80 degrees, FOV 207 mm, acquisition matrix 86, reconstruction matrix 86, in-plane resolution 2.41 mm by 2.41 mm, 52 slices, slice thickness 2.4 mm, bandwidth 2004 Hz/pixel, echo spacing 0.62 ms, 300 volumes per 10 minutes). Two opposite phase encoded spin-echo field maps were also acquired for distortion correction (interleaved series, A-P and P-A phase encoding, TR 9.5 s, TE 66 ms, FOV 207 mm, acquisition matrix 86, reconstruction matrix 86, in-plane resolution 2.41 mm by 2.41 mm, 52 slices, slice thickness 2.4 mm, bandwidth 1162 Hz/pixel, echo spacing 0.96, EPI factor 86).

SUPPLEMENTARY METHODS—Functional MRI Processing: The functional MRI processing pipeline employed AFNI (24.0.04), ANIMA (3.0), ANTS (2.3.1), FreeSurfer (6.0.3), and SPM12 (r7219) software packages. Processing was completed in 9 steps: reduction in BOLD series outlier voxel signals (despiking with AFNI); echo-planar imaging BOLD image distortion correction utilizing opposing phase encoded spin echo field-maps (ANIMA); BOLD time series spatial realignment to the 10th volume (SPM12); BOLD time series slice time correction (SPM12); high resolution anatomical T1 image removal of ultrasonic device and brain extraction (FreeSurfer); co-registration of high resolution T1 image to mean of realigned time series (SPM12); normalization of co-registered T1 image to MNI space (ANTS); normalization of BOLD time series by application of T1 image normalization deformation fields (ANTS); and spatial smoothing of BOLD time series with 8-mm Gaussian kernel (SPM12). First-level individual analyses were conducted with SPM12 using a whole-brain general linear model (GLM) with the canonical hemodynamic response function. Individual block-design analyses contrasted 5 interleaved epochs of stimulation with 5 epochs of rest across the 10-minute scan with two directional t-tests. Because the patient's head was fixed in place during the scan with an individually fitted mask, movement parameters were not included in the first-level block-design model in order to increase model sensitivity to ultrasound-induced activation changes.

SUPPLEMENTARY METHODS—Measurement of Target Engagement: Due to experimental troubleshooting, valid functional imaging data were available from 16 of 21 participants who received active sonication during functional MRI. Ten scans came from the 2nd treatment visit, 3 came from the 1^{st} treatment visit, and 3 came from an optional 3rd treatment visit. These discrepancies arose from attempts to optimize signal to noise in the fMRI experiment at two different timepoints during the study. These unsuccessful experimental changes aimed to mitigate potential confounds caused by ultrasonic device cabling and current flow in proximity to the MRI. Thus 5 participants were excluded from MRI analysis due to altered MRI experimental design. Data from the 16 included participants used identical experimental procedures.

SUPPLEMENTARY RESULTS—Secondary Clinical Outcomes: Mood changes were measured by administering the PANAS-X before and after each ultrasound treatment session. Each PANAS-X sub-scale was rescaled to a range of 0-100 and change in each sub-scale was computed. FIG. 27 shows the change for each sub-scale at the first treatment visit for active and sham treatment groups.

The secondary efficacy outcomes were the IDS-SR (including QIDS-SR) and GAD-7, administered immediately before each stimulation session and 7 days later. Changes in IDS-SR, QIDS-SR, and GAD-7 at 7 days were calculated, and two-sample t-tests were applied to test for group differences at each time point. In the intent-to-treat sample, no statistically significant differences between active and sham stimulation groups were found for the QIDS-SR (p=0.84, t(20)=−0.20, d=−0.08) or for the GAD-7 (p=0.52, t(20)=−0.66, d=0.28). Similarly, for the per-protocol sample, group differences were not significant (QIDS-SR: p=0.55, t(17)=−0.61, d=−0.28; GAD-7: p=0.29, t(17)=0.29, d=0.14).

TABLE 10

| Treatment | Day 0 | Day 0 | Day 7 | Day 7 |
|---|---|---|---|---|
| Group | (pre) | (post) | (pre) | (post) |
| Intent-to-treat | | | | |
| sample (n = 22) | | | | |
| Real (Day 0), | 55.5 (20.5) | 22.8 (17.5) | 31.1 (18.3) | 31.0 (26.6) |
| Sham (Day 7) | | | | |
| Sham (Day 0), | 40.0 (27.8) | 21.2 (22.3) | 30.4 (34.4) | 20.0 (36.1) |
| Real (Day 7) | | | | |
| Per-protocol | | | | |
| sample (n = 19) | | | | |
| Real (Day 0), | 56.1 (21.6) | 21.2 (18.1) | 30.0 (19.3) | 30.0 (28.1) |
| Sham (Day 7) | | | | |
| Sham (Day 0), | 39.5 (30.5) | 24.0 (23.3) | 34.0 (36.9) | 24.4 (38.8) |
| Real (Day 7) | | | | |

Table 10 is a table of Sadness scores at each time point. The extended Positive and Negative Affect Schedule (PANAS-X) was assessed pre-stimulation and post-stimulation at the first stimulation visit (Day 0) and the second stimulation visit (Day 7), as shown in FIG. 23. Values represent mean (SD) for the Sadness sub-scale

TABLE 11

| Treatment | Day 0 | | Day 7 | | |
|---|---|---|---|---|---|
| Group | (pre) | Day 1 | (pre) | Day 8 | Day 14 |
| Intent-to-treat | | | | | |
| sample (n = 22) | | | | | |
| Real (Day 0), | 11.4 (2.2) | 5.2 (4.5) | 6.3 (4.5) | 4.0 (4.6) | 5.2 (5.3) |
| Sham (Day 7) | | | | | |
| Sham (Day 0), | 10.8 (2.3) | 7.0 (3.8) | 7.2 (4.7) | 4.0 (4.5) | 6.6 (5.0) |
| Real (Day 7) | | | | | |
| Per-protocol | | | | | |
| sample (n = 19) | | | | | |
| Real (Day 0), | 11.9 (1.7) | 5.3 (4.8) | 6.1 (4.8) | 3.6 (4.8) | 4.7 (5.4) |
| Sham (Day 7) | | | | | |
| Sham (Day 0), | 10.7 (2.5) | 8.2 (2.9) | 7.6 (5.0) | 4.4 (4.9) | 7.7 (4.9) |
| Real (Day 7) | | | | | |

Table 11 is a table of Depression scores at each time point. The 6-item Hamilton Depression Rating Scale (HDRS-6) was administered pre-stimulation at the first stimulation visit (Day 0), 24 hours after the first stimulation (Day 1), pre-stimulation at the second stimulation visit (Day 7), 24 hours after the second stimulation (Day 8), and 7 days after the second stimulation (Day 14), as shown in FIG. 23. Values represent mean (SD) for HDRS-6 total score.

SUPPLEMENTARY RESULTS—Post-Crossover Outcomes: A randomized cross-over design was chosen so that each subject would receive both real and sham stimulation, thus maximizing the amount of efficacy, tolerability, and safety information collected from a limited subject pool. PANAS-X Sadness scores and HDRS-6 scores at each time point before and after the cross-over are shown in Table 10 and Table 11.

System 10 and its components can be configured and used as described in reference to study S5 and otherwise herein, such as to treat a patient as described in reference to study S5 and otherwise herein, such as to achieve the benefits of treatment achieved using system 10 as described in reference to study S5 and otherwise herein.

A patient suffering from a psychiatric disorder and selected for treatment using system 10 can comprise a patient diagnosed with treatment resistant depression. The patient selected for treatment using system 10 can have a primary DSM-5 diagnosis of major depressive disorder or bipolar disorder. The patient selected for treatment using system 10 can have, for at least 1 month or at least 2 months, a current moderate-to-severe depressive episode without psychotic features. The patient selected for treatment using system 10 can have a QIDS score of at least 6, or at least 10. The target location (e.g., for ultrasound delivery and/or ultrasound plus magnetic field energy delivery) can comprise a location of the subcallosal cingulate cortex (SCC). The target location can comprise at least 2 target locations, or at least 3 target locations within the SCC. At least 2 target locations can comprise midline target locations of the SCC. At least two target locations can be stimulated sequentially. At least 2 target locations can comprise a first target location, a second target location, and a third target location, and the first target location can be at least 2 mm and/or no more than 6 mm anterior to the second target location, and the third target location can be at least 2 mm posterior and/or no more than 6 mm posterior to the second target location. The first target location can be approximately 4 mm anterior to the second target location, and the third target location can be approximately 4 mm posterior to the second target location. The energy delivery can comprise a delivery of energy with a peak intensity of no more than 300 W/cm². The energy delivery can comprise a delivery of energy with a peak intensity of no more than 225 W/cm², and/or no more than 190 W/cm². The energy delivery can comprise a delivery of energy of no more than 3 hours, no more than 2 hours, and/or no more than 1 hour. The energy delivery can comprise a delivery of energy with an amplitude of at least 0.5 MPa, such as an energy delivery with an amplitude of approximately 1 MPa. The energy delivery can comprise a delivery of energy with field dimensions less than 5 mm by 5 mm by 40 mm, such as an energy delivery with field dimensions of approximately 2.4 mm by 3.6 mm by 20.4 mm. The energy delivery can comprise a delivery of energy comprising bursts of at least 10 msec in duration that can be delivered at less than 90% duty cycle. The bursts can be separated by a burst interval of no more than 20 seconds. The energy delivery can comprise a delivery of energy comprising bursts of approximately: 30 msec in duration; 50% duty cycle; and 0.7 second burst interval. The energy delivery can comprise a delivery of energy comprising bursts of approximately: 30 msec in duration; 50% duty cycle; and 1.4 second burst interval. The energy delivery can comprise a delivery of energy for at least 30 seconds. The energy delivery can comprise a delivery of energy for approximately 60 seconds. The energy delivery can comprise a delivery of energy in blocks comprising rest epochs and active sonication epochs. The rest epochs and active epochs can be interleaved. The active epochs and/or the rest epochs can comprise a duration of at least 30 seconds, such as a duration of approximately 1 minute, or approximately 3 minutes. Each block can comprise at least 6 total epochs, such as approximately 10 total epochs. At least 2 blocks of energy can be delivered. The energy delivery parameters can vary between blocks. The energy delivery duration can vary between blocks. At least 3 blocks of energy can be delivered. Each block can comprise at least 3 test energy deliveries of at least one-minute in duration. Each block can comprise approximately: 3 to 5 one minute energy deliveries (e.g., sonications) with 1.5 second burst intervals, such as to test the tolerability of each target location. Each block can comprise at least 6 energy deliveries of at least two minutes in duration. Each block can comprise approximately: 6 three-minute sonications with 1.4 second burst intervals, and/or 6 three-minute sonications with 0.7 second burst intervals. The treatment provided by system 10 can achieve at least a 50% change in Sadness, such as approximately a 63% change in sadness. The treatment provided by system 10 can achieve a change in HRDS-6 of at least 25% at day 1 after the energy delivery. The treatment provided by system 10 can achieve a change in HRDS-6 of approximately 55% at day 1 after the energy delivery. The treatment provided by system 10 can achieve a change in HRDS-6 of at least 20% at day 7 after the energy delivery. The treatment provided by system 10 can achieve a change in HRDS-6 of approximately 52% at day 7 after the energy delivery.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the inventive concepts, which are defined in the accompanying claims.

1. A system for delivering energy to a patient, the system comprising:
a treatment device comprising one or more energy delivery elements, wherein the treatment device is configured to deliver ultrasound energy to a target location of the patient via the one or more energy delivery elements,
wherein the energy delivery to the target location treats a medical condition of the patient.

2. The system according to claim 1, wherein the target location comprises a location of the brain of the patient.

3. The system according to claim 1, wherein the treatment device is configured to deliver energy to an anatomical location of the patient selected from the group consisting of: brain; heart; liver; pancreas; spleen; dorsal root ganglia; spinal cord; peripheral nerves; and combinations thereof.

4. (canceled)

5. The system according to claim 1, wherein the treatment device is configured to further deliver magnetic field energy.

6. The system according to claim 5, wherein the magnetic field comprises a magnetic field of at least 0.5 T.

7. The system according to claim 1, wherein the treatment device is configured to deliver one, two, or more energies selected from the group consisting of: sound energy, such as ultrasound energy; light energy, such as laser light energy; thermal energy, such as heat energy and/or cryogenic energy; electromagnetic energy, such as radiofrequency energy, microwave energy, and/or electroporation energy; chemical energy; mechanical energy; and combinations thereof.

8. The system according to claim 1, wherein the target location spans at most 10 mm, and wherein the system is configured to accurately deliver the energy into the target location.

9. The system according to claim 8, wherein the target location spans at most 5 mm.

10. The system according to claim 1, wherein the treatment device is configured to deliver the energy with a spatial resolution of no more than 1.0 mm and a temporal accuracy of no more than 5 μs.

11. The system according to claim 1, wherein the one or more energy delivery elements comprise multiple energy delivery elements arranged in one or more spherically focused arrays.

12. The system according to claim 11, wherein the one or more spherically focused arrays each comprise a radius of at least 10 mm, no more than 5,000 mm, or both.

13. The system according to claim 1, wherein the treatment device comprises a first treatment assembly configured to deliver energy and to be positioned on the right side of the head of the patient and a second treatment assembly configured to deliver energy and to be positioned over the left side of the head of the patient.

14. The system according to claim 13, further comprising a housing configured to position the first and second treatment assemblies to each deliver the energy through the temporal bone window of the patient's skull.

15. The system according to claim 1, wherein the system is configured to deliver energy to multiple different target locations of the deep brain without moving the treatment device and without moving the patient's head.

16. The system according to claim 15, wherein the system is configured to deliver energy to at least a first target location and a second target location in a sequential arrangement.

17. The system according to claim 1, wherein the system comprises a housing and a patient mask that are collectively configured to reproducibly position the patient's head in a desired arrangement for delivery of energy without use of MRI for guidance.

18. (canceled)

19. (canceled)

20. The system according to claim 1, further comprising an algorithm wherein the system is configured to determine energy delivery drive signals that compensate for one or more obstacles present in the path of energy delivery from the treatment device to the target location.

21. The system according to claim 20, wherein the one or more obstacles comprise an obstacle selected from the group consisting of: the skull; the scalp; hair; a component of the system; and combinations thereof.

22-128. (canceled)
